SEQUENCE LISTING (2 sequences)

SEQ ID NO: 1 - DNA, 12 bp, synthetic construct
  source 1..12: mol_type = other DNA
tatggtcatcga

SEQ ID NO: 2 - DNA, 12 bp, synthetic construct
  source 1..12: mol_type = other DNA
tatggtcgtcga

(19) United States
(12) Patent Application Publication
(10) Pub. No.: US 2025/0084475 A1
(43) Pub. Date: Mar. 13, 2025

(54) SEQUENCING SYSTEMS AND METHODS

(71) Applicant: ULTIMA GENOMICS, INC., Fremont, CA (US)

(72) Inventors: Florian OBERSTRASS, Redwood City, CA (US); Gilad ALMOGY, Palo Alto, CA (US); Ron Saar DOVER, Palo Alto, CA (US); Na Kyung LEE, East Palo Alto, CA (US); Jillynne QUINN, Livermore, CA (US)

(21) Appl. No.: 18/828,961

(22) Filed: Sep. 9, 2024

Related U.S. Application Data
(60) Provisional application No. 63/581,542, filed on Sep. 8, 2023.
(60) Provisional application No. 63/563,265, filed on Mar. 8, 2024.
(60) Provisional application No. 63/602,255, filed on Nov. 22, 2023.

(51) Int. Cl. C12Q 1/6874

(52) U.S. Cl. CPC C12Q 1/6874

(57) ABSTRACT

Methods of sequencing and resequencing nucleic acids using incorporation and binding of nucleotides, including terminated nucleotides, are provided. Also provided herein are methods using combinations of terminated nucleotides and non-terminated nucleotides or combinations of terminated nucleotides and non-incorporable nucleotides. These methods allow for determination of the length of homopolymer sequences and increased accuracy of the sequencing reads. The compositions, reagents, and kits for practicing the methods are also provided.

### CROSS REFERENCE

This application claims the benefit of U.S. Provisional Pat. App. Nos. 63/581,542, filed on Sep. 8, 2023, 63/563,265, filed on Mar. 8, 2024, and 63/602,255, filed on Nov. 22, 2023, each of which is entirely incorporated by reference herein for all purposes.

### SEQUENCE LISTING

The instant application contains a Sequence Listing which has been submitted electronically in XML format and is hereby incorporated by reference in its entirety. Said XML copy, created on Oct. 26, 2024, is named 51024-782_201_SL.xml and is 2.64 kilobytes in size.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

### SUMMARY

The present disclosure provides methods, compositions, kits, and/or systems for library preparation, analyte detection, and sequencing. Analyte detection may comprise target nucleic acid detection. Target nucleic acid detection may comprise nucleic acid sequencing reactions. Sequencing reactions may comprise single molecule sequencing or colony sequencing. Sequencing reactions may comprise paired end sequencing. The present disclosure may be advantageous to improve sequencing results.

In an aspect, a method is provided comprising: (a) contacting a growing strand hybridized to a template with a first reagent mixture comprising labeled, non-terminated bases and reversibly terminated bases of a first same canonical base type and detecting a first signal indicative of incorporation of at least a subset of the labeled, non-terminated bases of the first reagent mixture in the growing strand, or lack thereof, to generate first sequencing data; (b) reversing termination of the reversibly terminated bases of the first reagent mixture incorporated in the growing strand, if any; (c) contacting the growing strand with a second reagent mixture comprising labeled, non-terminated bases and terminated bases of the first same canonical base type and detecting a second signal indicative of incorporation of at least a subset of the labeled, non-terminated bases of the second reagent mixture in the growing strand, or lack thereof, to generate second sequencing data; and (d) processing the first sequencing data and the second sequencing data to determine length information of a homopolymer sequence in the template.

In some embodiments, the length information of the homopolymer sequence in the template comprises a minimum length of the homopolymer sequence. In some embodiments, the length information of the homopolymer sequence in the template comprises a total length of the homopolymer sequence.

In some embodiments, the method further comprises (e) reversing termination of the reversibly terminated bases of the second reagent mixture incorporated in the growing strand, if any, and (f) contacting the growing strand with a third reagent mixture comprising unlabeled, non-terminated bases of the first same canonical base type.

In some embodiments, the method further comprises (g) repeating (a)-(f) with a second same canonical base type different from the first canonical base type. In some embodiments, the method further comprises (h) repeating (a)-(f) with a third same canonical base type different from the first canonical base type and the second canonical base type. In some embodiments, the method further comprises (i) repeating (a)-(f) with a fourth same canonical base type different from the first canonical base type, the second canonical base type, and the third canonical base type. In some embodiments, the method further comprises repeating (a)-(i) at least 10 times.

In some embodiments, the first signal is localized to a single molecule of the template. In some embodiments, the first signal is localized to a colony of molecules comprising the template.

In some embodiments, the template is immobilized to a substrate surface. In some embodiments, the template is coupled to a bead that is immobilized to the substrate surface.

In some embodiments, the substrate surface comprises at least 1,000,000 individually addressable locations and the template is immobilized to an individually addressable location in the at least 1,000,000 individually addressable locations.

In another aspect, a method of sequencing a template molecule is provided comprising: (a) hybridizing a primer to a primer binding site on the template molecule; (b) extending the primer through a first region of the template molecule, wherein the extending comprises alternatively adding nucleotides and detecting incorporation of nucleotides into the extending primer; (c) denaturing the extended primer from the template molecule; (d) hybridizing a primer to the primer binding site on the template molecule; (e) extending the primer through the first region of the template molecule without detecting incorporation of nucleotides; and (f) extending the primer through a second region of the template molecule, wherein the extending comprises alternatively adding nucleotides and detecting incorporation of nucleotides into the extending primer.

In some embodiments, the nucleotides added during the extending (b) and (f) comprise reversibly terminated, labeled nucleotides.

In some embodiments, the nucleotides added during the extending (e) comprise unlabeled nucleotides. In some embodiments, the nucleotides added during the extending (e) comprise labeled nucleotides. In some embodiments, the nucleotides added during the extending (e) comprise unterminated nucleotides.

In some embodiments, the alternatively adding nucleotides and detecting incorporation of nucleotides in the extending (b) and (f) comprises, in each step, adding nucleotides of more than one base type. In some embodiments, the method further comprises adding nucleotides of the four canonical base types. In some embodiments, nucleotides of each base type are differently labeled. In some embodiments, a nucleotide of a first base type is labeled with a first fluorophore and a nucleotide of a second base type is labeled with a second fluorophore. In some embodiments, at least one base type of nucleotide is unlabeled. In some embodiments, at least a subset of the nucleotides added during the extending (b) and (f) comprise unlabeled and/or unterminated nucleotides.

In some embodiments, the extending (b) and (f) further comprise, after detecting incorporation of nucleotides, cleaving reversible terminators from incorporated nucleotides.

In another aspect, a method of sequencing a template molecule is provided comprising: (a) hybridizing a primer to a first primer binding site on the template molecule; (b) extending the primer through a first region of the template molecule, wherein the extending comprises alternatively adding nucleotides and detecting incorporation of nucleotides; (c) extending the primer through a second region of the template molecule, thereby producing a copied template molecule, wherein the extending comprises adding nucleotides of at least one base type and, at one or more time points, detecting incorporation of nucleotides; (d) denaturing the copied template molecule from the template molecule; (e) hybridizing a primer to a second primer binding site on the copied template molecule; (f) extending the primer through a first region of the copied template molecule, wherein the extending comprises alternatively adding nucleotides and detecting incorporation of nucleotides; and (g) extending the primer through a second region of the copied template molecule, wherein the extending comprises adding nucleotides of at least one base type and, at one or more time points, detecting incorporation of nucleotides.

In some embodiments, the extending (c) and (g) comprises, in one or more steps, adding nucleotides of two base types. In some embodiments, the extending (c) and (g) comprises, in one or more steps, adding nucleotides of three base types. In some embodiments, the extending (c) and (g) comprises, in one or more steps, adding nucleotides of four base types.

In some embodiments, the sequence of the first region of the template molecule is determined from detection of nucleotide incorporation in the extending (b) and by at least one detection of nucleotide incorporation in the extending (f). In some embodiments, the sequence of the second region of the template molecule is determined from detection of nucleotide incorporation in the extending (f) and by at least one detection of nucleotide incorporation in the extending (b).

In some embodiments, each detection determines a base type of the respective incorporated nucleotide. In some embodiments, each detection further comprises a confidence value of a respective nucleotide incorporation.

In some embodiments, the first primer binding site is at 3′ end of the template molecule. In some embodiments, the second primer binding site is at 3′ end of the copied template molecule. In some embodiments, the template molecule and the copied template molecule are each single-stranded.

In some embodiments, the nucleotides added during the extending (b) and (f) comprise reversibly terminated, labeled nucleotides. In some embodiments, the nucleotides added during the extending (c) and (g) comprise a first subset of unlabeled nucleotides and a second subset of labeled nucleotides. In some embodiments, the nucleotides added during the extending (c) and (g) comprise labeled nucleotides. In some embodiments, the nucleotides added during the extending (c) and (g) comprise unterminated nucleotides. In some embodiments, at least a subset of the nucleotides added during the extending (b) and (f) comprise unlabeled and/or unterminated nucleotides.

In some embodiments, the extending (b) and (f) further comprise, after detecting incorporation of nucleotides, cleaving reversible terminators from incorporated nucleotides.

In another aspect, a method of sequencing a nucleic acid molecule comprises hybridizing the nucleic acid molecule to a primer to form a hybridized template; extending the primer using labeled, terminated nucleotides provided in multiple flows comprising four nucleotide base types; detecting a signal from an incorporated labeled nucleotide or an absence of a signal as the primer is extended by the nucleotide flows, denaturing the extended primer; hybridizing another primer to the nucleic acid molecule to a primer to reform the hybridized template; extending the primer using unterminated nucleotide provided in separate nucleotide flows according to a repeated flow-cycle order comprising four or more separate nucleotide flows; further extending the primer using labeled, terminated nucleotides provided in multiple flows comprising four nucleotide base types; and detecting a signal from an incorporated labeled nucleotide or an absence of a signal as the primer is extended by the nucleotide flows.

In another aspect, a method is provided, comprising: (a) providing a plurality of template molecules, wherein the template molecule comprise primers hybridized thereto; (b) contacting the plurality of template molecules with a reaction mixture comprising a plurality of nucleotides under conditions sufficient for one or more nucleotides to be incorporated into one or more primers hybridized to template molecules and for one or more nucleotides to be transiently bound to one or more template molecules; and (c).

In some embodiments, the method further comprises: (d) removing transiently bound nucleotides; and (e) detecting an additional signal, wherein the additional signal indicates the presence or absence of incorporated nucleotides.

In another aspect, a method is provided, comprising: (a) providing a plurality of template molecules with primers hybridized thereto; (b) contacting the plurality of template molecules with a reaction mixture comprising terminated and unterminated nucleotides, wherein a first subset of the plurality of template molecules incorporate a terminated nucleotide and a second subset of the plurality of template molecules transiently bind to an unterminated nucleotide; (c) detecting a signal, wherein the signal indicates the presence or absence of incorporated or transiently bound nucleotides; (d) removing transiently bound nucleotides; and (e) detecting an additional signal, wherein the additional signal indicates the presence or absence of incorporated nucleotides

In some embodiments, the reaction mixture further comprises a polymerase that can incorporate terminated nucleotides and transiently bind unterminated nucleotides.

In some embodiments, the reaction mixture further comprises a first polymerase that can incorporate terminated nucleotides and a second polymerase that can transiently bind unterminated nucleotides. In some embodiments, the reaction mixture further comprises a polymerase that can incorporate nucleotides and catalyze transient binding of nucleotides. In some embodiments, the transiently bound nucleotides are non-incorporable nucleotides. In some embodiments, the incorporated nucleotides are reversibly terminated nucleotides. In some embodiments, the reaction mixture comprises: a nucleotide of a first base type comprising a first label type; a nucleotide of a second base type comprising a second label type; a nucleotide of a third base type comprising the first label type; and a nucleotide of a fourth base type comprising the second label type. In some embodiments, nucleotides of the first and second base types are reversibly terminated. In some embodiments, nucleotides of the third and fourth base types are non-incorporable.

In some embodiments, the reaction mixture comprises: a nucleotide of a first base type comprising a first label type; a nucleotide of a second base type comprising a second label type; a nucleotide of a third base type; and a nucleotide of a fourth base type comprising the second label type. In some embodiments, the nucleotide of the third base type does not comprise a label. In some embodiments, nucleotides of the first, second, and third base types are reversibly terminated. In some embodiments, nucleotides of the fourth base type are non-incorporable.

In some embodiments, the method further comprises, after the detecting (e): (f) contacting the plurality of template molecules with another reaction mixture comprising a plurality of terminated nucleotides; and (g) cleaving reversible terminator and labeling moieties. In some embodiments, the method further comprises repeating steps (a)-(g) one or more times to determine the sequences of the template molecules.

In another aspect, a method for identifying a sequencing is provided, comprising: (a) providing a template molecule with a primer hybridized thereto; (b) performing at least two repeated cycles of: i) incorporation or transient binding of nucleotides to the template molecule; ii) detection of a signal, wherein the signal indicates the presence or absence of incorporated or transiently bound nucleotides; and iii) cleavage of reversible terminator and labeling moieties; and (c) determining the sequence of the template molecule based on the detection of labeled nucleotides.

In some embodiments, the at least two repeated cycles further comprise providing a reaction mixture comprising: a nucleotide of a first base type that comprising a first label type; a nucleotide of a second base type comprising a second label type; a nucleotide of a third base type comprising the first label type; and a nucleotide of a fourth base type comprising the second label type. In some embodiments, nucleotides of the first and second base types are reversibly terminated. In some embodiments, nucleotides of the third and fourth base types are non-incorporable.

In some embodiments, the method further comprises, prior to cleaving (iii): (iv) washing the template molecule to remove transiently bound nucleotides; and (v) detecting an additional signal, wherein the additional signal indicates the presence or absence of incorporated or transiently bound nucleotides. In some embodiments, the method further comprises, prior to cleaving (iii): (vi) contacting the template molecule with another reaction mixture comprising a plurality of terminated nucleotides.

In another aspect, a method is provided comprising: (a) providing a plurality of template molecules with primers hybridized thereto; (b) contacting the plurality of template molecules with a first reaction mixture comprising terminated and unterminated nucleotides, wherein a first subset of the plurality of template molecules incorporate a terminated nucleotide and a second subset of the plurality of template molecules transiently bind to an unterminated nucleotide; (c) detecting a signal, wherein the signal indicates the presence or absence of incorporated or transiently bound nucleotides; (d) removing transiently bound nucleotides; (e) contacting the plurality of template molecules with a second reaction mixture comprising unterminated nucleotides that may transiently bind template molecules; and (f) detecting an additional signal, wherein the additional signal indicates the presence or absence of incorporated or transiently bound nucleotides.

In some embodiments, the first reaction mixture further comprises a polymerase that can incorporate terminated nucleotides and transiently bind unterminated nucleotides. In some embodiments, the first reaction mixture further comprises a first polymerase that can incorporate terminated nucleotides and a second polymerase that can transiently bind unterminated nucleotides. In some embodiments, the transiently bound nucleotides are non-incorporable nucleotides. In some embodiments, the reaction mixture comprises: a nucleotide of a first base type comprising a first label type; a nucleotide of a second base type; a nucleotide of a third base type comprising the first label type; and a nucleotide of a fourth base type comprising a second label type.

In another aspect, a method of sequencing is provided, comprising: (a) providing a template molecule comprising a primer hybridized thereto, wherein the primer is not blocked for nucleotide incorporation; (b) contacting the template molecule with a first reaction mixture comprising a plurality of nucleotides under conditions sufficient to permit binding of non-incorporable nucleotides to the template molecule, wherein the plurality of nucleotides comprises at least one nucleotide base type; and (c) detecting a signal, wherein the signal indicates binding or lack thereof of a nucleotide analog to the template molecule.

In some embodiments, the method further comprises, after the detecting (c), washing the template molecule to remove non-incorporable nucleotides. In some embodiments, the binding of non-incorporable nucleotides comprises the formation of hydrogen bonds. In some embodiments, the binding of non-incorporable nucleotides is transient. In some embodiments, the method further comprises contacting the template molecules with a second reaction mixture comprising a plurality of terminated nucleotides under conditions sufficient to permit incorporation of nucleotides into the primer hybridized to the template molecule. In some embodiments, the method further comprises cleavage of reversible terminators from incorporated nucleotides. In some embodiments, the method further comprises repeating steps (a)-(e) one or more times to determine the sequence of the template molecule.

In some embodiments, the first reaction mixture further comprises a first polymerase to enable binding of non-incorporable nucleotides to the template molecule, and the second reaction mixture comprises a second polymerase to enable incorporation of terminated nucleotides into the primer. In some embodiments, the first and second polymerase are the same. In some embodiments, the first and second polymerase are different. In some embodiments, the first reaction mixture comprises: a nucleotide of a first base type comprising a first label type; a nucleotide of a second base type; a nucleotide of a third base type comprising the first label type; and a nucleotide of a fourth base type comprising a second label type. In some embodiments, the nucleotide of the second base type does not comprise a label. In some embodiments, the first reaction mixture does not comprise a cation that inhibits nucleotide incorporation. In some embodiments, the first reaction mixture comprises a polymerase that exhibits exonuclease activity.

In some embodiments, the template molecule is a nucleic acid molecule. In some embodiments, the nucleic acid molecule is DNA or RNA. In some embodiments, the template molecule is amplified. In some embodiments, the template molecule is a DNA nanoball. In some embodiments, the template molecule is not amplified.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example workflow for processing a sample for sequencing.

FIG. 2 illustrates an example flow sequencing method that can be used to generate the sequencing data described herein.

FIG. 3 illustrates an example flowgram.

FIG. 4 illustrates examples of individually addressable locations distributed on substrates, as described herein.

FIGS. 5A-5B each illustrate multiplexed stations in a sequencing system.

FIG. 6 illustrates a computer system that is programmed or otherwise configured to implement methods provided herein.

FIG. 7 shows an example image of a substrate with a hexagonal lattice of beads, as described herein.

FIGS. 8A-8E illustrate exemplary sequencing methods.

FIG. 9A illustrates a mixed-reversibly terminated sequencing method.

FIGS. 9B and 9C illustrate mixed-color sequencing methods.

FIG. 10 illustrates a paired end sequencing method.

FIG. 11A shows two example sequences (e.g., extended sequencing primer sequences) that differ at least at one locus. Sequence 1 comprises TATGGTCATCGA (SEQ ID NO: 1) and Sequence 2 comprises TATGGTCGTCGA (SEQ ID NO: 2).

FIG. 11B illustrates sequencing data, using one flow order, for two different extended sequences, SEQ ID NO: 1 and SEQ ID NO: 2.

FIG. 11C illustrates sequencing data, using two different flow orders, for two different extended sequences, SEQ ID NO: 1 and SEQ ID NO: 2.

FIG. 12 illustrate a non-limiting example of paired end sequencing.

FIG. 13 illustrate a non-limiting example of paired end sequencing.

FIG. 14 illustrate a non-limiting example of paired end sequencing.

FIG. 15 illustrate a non-limiting example of paired end sequencing.

FIG. 16 illustrate a non-limiting example of paired end sequencing.

FIG. 17 illustrate a non-limiting example of paired end sequencing.

FIG. 18 illustrate a non-limiting example of paired end sequencing.

FIG. 19 illustrate a non-limiting example of end sequencing.

FIG. 20 illustrate a non-limiting example of paired end sequencing.

FIG. 21 shows structures of exemplary non-incorporable nucleotide analogs.

FIGS. 22A-22D illustrate examples of nucleotide flows for sequencing with reversibly terminated and non-incorporable nucleotides. In FIG. 22A, a combination of labeled non-incorporable and labeled reversibly terminated nucleotide base types are used. In FIG. 22B, at least one reversibly terminated nucleotide base type is unlabeled. In FIG. 22C, non-incorporable nucleotides are used, where each nucleotide base type is labeled with a different label type. In FIG. 22D, non-incorporable nucleotides are used, where three nucleotide base types are labeled, and where each labeled nucleotide base type is labeled with a different label type.

FIG. 23 shows, for four exemplary flow cycle orders (including 3 that are extended flow cycle orders), the sensitivity of detecting a SNP permutation given random sequencing start positions.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “analyte,” as used herein, generally refers to an object that is the subject of analysis, or an object, regardless of being the subject of analysis, which is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a sample derived from a subject or specimen.

As used herein, the terms “template nucleic acid” or “target nucleic acid” generally refer to the nucleic acid to be sequenced. The template nucleic acid may be an analyte or be associated with an analyte. For example, the analyte can be a mRNA, and the template nucleic acid is the mRNA, or a cDNA derived from the mRNA, or other derivative thereof. In another example, the analyte can be a protein, and the template nucleic acid is an oligonucleotide that is conjugated to an antibody that binds to the protein, or derivative thereof. Examples of sequencing include single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide,” as used herein, generally refers to any nucleotide or nucleotide analog. The nucleotide may be naturally occurring or non-naturally occurring. The nucleotide may be a non-standard, modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). The nucleotide may be non-terminated (e.g., natural or modified). In some cases, nucleotides may include modifications in their phosphate moieties, including modifications to a triphosphate moiety. Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for detection.

The term “sequencing,” as used herein, generally refers to a process for generating or identifying a sequence of a biological molecule, such as a nucleic acid. The sequence may be a nucleic acid sequence which comprises a sequence of nucleic acid bases. Examples of sequencing include single molecule sequencing or sequencing by synthesis, for example. Sequencing may comprise generating sequencing signals and/or sequencing reads.

The term “terminator” as used herein with respect to a nucleotide may generally refer to a moiety that is capable of terminating primer extension. A terminator may be a reversible terminator. A reversible terminator may comprise a blocking or capping group that is attached to the 3′-oxygen atom of a sugar moiety (e.g., a pentose) of a nucleotide or nucleotide analog. Such moieties are referred to as 3′-O-blocked reversible terminators. In some cases, a blocking group may be an azidomethyl or disulfide blocking group. Examples of 3′-O-blocked reversible terminators include, for example, 3′-O-(2-nitrobenzyl) reversible terminators, 3′-ONH₂ reversible terminators, 3′-O-(2-cyanoethyl) reversible terminators, 3′-O-allyl reversible terminators, and 3′-O-aziomethyl reversible terminators. 3′-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp. and the “lightning terminator” developed by Michael L. Metzker et al. A reversible terminator may comprise a blocking group in a linker (e.g., a cleavable linker) and/or dye moiety of a nucleotide analog. The blocking groups may be attached to the nucleotide via a cleavable linker. In some instances, the blocking groups may comprise a reporter moiety (e.g., dye moiety). Alternatively, the reporter moiety may be attached to the nucleotide at a different location (e.g., at a nucleobase) via an independent linker. In some instances, the linker for the blocking group and the linker for the dye may be the same type of linker and/or otherwise be cleavable via the same stimulus (e.g., cleaving agent). Cleavable linkers can include, for example, disulfide linkers and fluoride-cleavable linkers. The reversibly terminated nucleotide may be unblocked, such as by cleaving the blocking group (e.g., using a cleaving reagent or irradiation), to reverse the termination. Unblocking may be facilitated by introducing one or more cleaving agents. The cleaving agent may be dependent on the unblocking group present. For example, reducing agents may be used to cleave disulfide bonds or other reductive cleavage groups. Reducing agents include, but are not limited to, phosphine compounds, water soluble phosphines, nitrogen containing phosphines and salts and derivatives thereof, dithioerythritol (DTE), dithiothreitol (DTT) (cis and trans isomers, respectively, of 2,3-dihydroxy-1,4-dithiolbutane), 2-mercaptoethanol or β-mercaptoethanol (BME), 2-mercaptoethanol or amino ethanethiol, glutathione, thioglycolate or thioglycolic acid, 2,3-dimercaptopropanol and tris (2-carboxyethyl) phosphine (TCEP), tris(hydroxymethyl) phosphine (THP) and p-[tris(hydroxymethyl) phosphine] propionic acid (THPP). A phosphine reagent may include triaryl phosphines, trialkyl phosphines, sulfonate containing and carboxylate containing phosphines and derivatized water soluble phosphines. In another example, for 2-cyanoethyl blocking groups and/or cyanoethyl ester linkers, fluoride ions (e.g., solution comprising tetrabutylammonium fluoride (TBAF), etc.) can be used as cleaving agents. See, e.g., Diana C. Knapp et al., Fluoride-Cleavable, Fluorescently Labelled Reversible Terminators: Synthesis and Use in Primer Extension, 17 CHEM. EUR. J. 2903-15 (2011), and Diana C. Knapp et al., Fluorescent Labeling of (Oligo) Nucleotides by a New Fluoride Cleavable Linker Capable of Versatile Attachment Modes, 21 BIOCONJUGATE CHEM. 1043-55 (2010), which are entirely incorporated herein by reference.

The terms “non-incorporable” or “non-incorporatable” as used with respect to a nucleotide may generally refer to a nucleotide that cannot be used as a substrate nucleotide to complete a nucleic acid polymerization by a polymerase. For example, a polymerase may not catalyze the polymerization reaction. In another example, the non-incorporable nucleotide may not allow a nucleic acid polymerization reaction to initiate or complete. In another example, the non-incorporable nucleotide may inhibit or prevent a forward nucleic acid polymerization reaction and/or activate or initiate a backward nucleic acid polymerization reaction. In some cases, the non-incorporable nucleotide may bind the polymerase, the polymerizing nucleic acid molecule (e.g., the extending primer molecule), the polymerization template molecule (e.g., the template nucleic acid molecule), or any combinations thereof. As used herein, the term “incorporable” nucleotide may generally refer to a nucleotide that may be incorporated to the polymerizing nucleic acid molecule by a polymerase during the nucleic acid polymerization reaction. As such, a nucleotide which is a non-incorporable nucleotide with respect to a first polymerase may be an incorporable nucleotide with respect to a second polymerase. In some cases, a non-incorporable nucleotide may be non-incorporable by any polymerase. In some cases, an incorporable nucleotide may be incorporable by any polymerase. In some cases, an incorporable nucleotide may also be a reversibly terminated nucleotide.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., an A-base-containing solution) to a sequencing reaction space at a first time point and (ii) providing a nucleotide-containing reagent (e.g., G-base-containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of base types (e.g., A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:

(e.g., [ATGCATGCATGATGATGATGCATGC]).

Such one-dimensional matrix or linear array of bases in the flow order may also be referred to herein as a “flow space.” A flow order may have any number of nucleotide flows. A “flow position,” as used herein, generally refers to the sequential position of a given nucleotide flow entry in the flow space (e.g., an element in the one-dimensional matrix or linear array). A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A T G C], [A A T T G G C C], [A T], [A/T A/G], [A A], [A], [A T G], etc.). A flow cycle may have any number of nucleotide flows. A given flow cycle may be repeated one or more times in the flow order, consecutively or non-consecutively. Accordingly, the term “flow cycle order,” as used herein, generally refers to an ordering of flow cycles within the flow order and can be expressed in units of flow cycles. For example, where [A T G C] is identified as a 1^{st} flow cycle, and [A T G] is identified as a 2^{nd} flow cycle, the flow order of [A T GCATGCATGATGATGATGCATG C] may be described as having a flow-cycle order of [1^{st} flow cycle; 1^{st} flow cycle; 2^{nd} flow cycle; 2^{nd} flow cycle; 2^{nd} flow cycle; 1^{st} flow cycle; 1^{st} flow cycle]. Alternatively or in addition, the flow cycle order may be described as [cycle 1, cycle, 2, cycle 3, cycle 4, cycle 5, cycle 6], where cycle 1 is the 1^{st} flow cycle, cycle 2 is the 1^{st} flow cycle, cycle 3 is the 2^{nd} flow cycle, etc. In some cases, a flow-cycle order may be [T GC A]. However, any other permutation of the nucleotides T (or U), G, C, and A may be used as a flow-cycle order.

### Sample Processing Methods

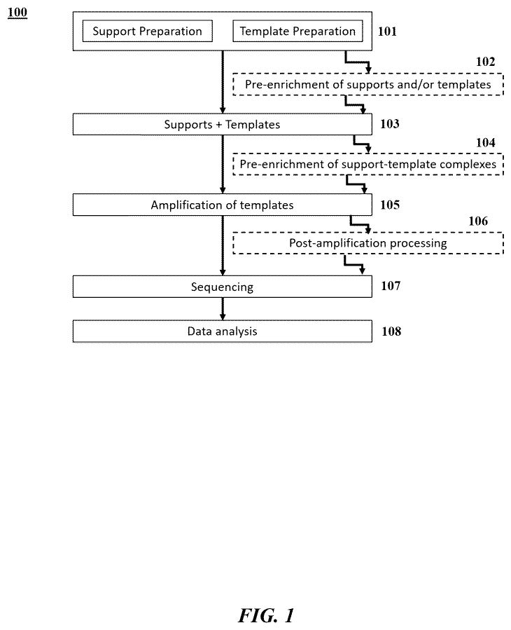

Described herein are devices, systems, methods, compositions, and kits for processing samples, such as to prepare a sample for sequencing, to sequence a sample, and/or to analyze sequencing data. FIG. 1 illustrates an example sequencing workflow 100, according to the devices, systems, methods, compositions, and kits of the present disclosure.

Supports and/or template nucleic acids may be provided and/or prepared (101) to be compatible with downstream sequencing operations (e.g., 107). A support (e.g., bead) may help immobilize a template nucleic acid to a substrate, such as when the template nucleic acid is coupled to the support, and the support is in turn immobilized to the substrate. The support may further function as a binding entity to retain derivatives molecules (e.g., amplification products) from a same template nucleic acid together for downstream processing, such as for sequencing operations. This may be useful in distinguishing a colony from other colonies (e.g., on other supports) and generating amplified sequencing signals corresponding to a template nucleic acid. A support may comprise an oligonucleotide comprising one or more functional nucleic acid sequences. The oligonucleotide may be single-stranded, double-stranded, or partially double-stranded. For example, the oligonucleotide may comprise a capture sequence, a primer sequence, a sequencing primer sequence, a barcode sequence, a sample index sequence, a unique molecular identifier (UMI), a flow cell adaptor sequence, an adaptor sequence, a target sequence, a random sequence, a binding sequence (e.g., for a splint, primer, template nucleic acid, capture sequence, etc.), or any other functional sequence useful for a downstream operation, a complement thereof, or any combination thereof. The capture sequence may be configured to hybridize to a sequence of a template nucleic acid or derivative thereof. The support may comprise a plurality of oligonucleotides, for example on the order of 10, 102, 103, 104, 105, 106, 107, or more molecules. The support may comprise a single species of oligonucleotide which comprise identical sequences. The support may comprise multiple species of oligonucleotides which have varying sequences. In some cases, the support comprises a single species of a primer (e.g., forward primer) for amplification. In some cases, the support comprises two species of primer (e.g., forward primer, reverse primer) for amplification. Devices, systems, methods, compositions, and kits for preparing and using support species are described in further detail in U.S. Patent Pub. No. 20220042072A1 and International Patent Pub. No. WO2022040557A2, each of which is entirely incorporated herein by reference for all purposes.

A support may comprise one or more capture entities, where a capture entity is configured for capture by a capturing entity. A capture entity may be coupled to or be part of an oligonucleotide coupled to the support. A capture entity may be coupled to or be part of the support. Examples of capture entity-capturing entity pairs and capturing entity-capture entity pairs include streptavidin (SA)-biotin; complementary sequences; magnetic particle-magnetic field system; charged particle-electric field system; azide-cyclooctyne; thiol-maleimide; click chemistry pairs; cross-linking pairs; etc. The capture entity-capturing entity pair may comprise one or more chemically modified bases. A capture entity and capturing entity may bind, couple, hybridize, or otherwise associate with each other. The association may comprise formation of a covalent bond, non-covalent bond, releasable bond (e.g., cleavable bond that is cleavable upon application of a stimulus), and/or no bond. The capture entity may be capable of linking to a nucleotide. In some instances, the capturing entity may comprise a secondary capture entity, for example, for subsequent capture by a secondary capturing entity. The secondary capture entity and secondary capturing entity may comprise any one or more of the capturing mechanisms described elsewhere herein.

A support may comprise one or more cleavable moieties, also referred to herein as excisable moieties. The cleavable moiety may be coupled to or be part of an oligonucleotide coupled to the support. The cleavable moiety may be coupled to the support. A cleavable moiety may comprise any useful moiety that can be used to cleave an oligonucleotide (or portion thereof) from the support, or otherwise release a nucleic acid strand from the support and/or the oligonucleotide. A cleavable moiety may comprise a uracil, a ribonucleotide, methylated nucleotide, or other modified nucleotide that is excisable or cleavable using an enzyme (e.g., UDG, RNAse, APE1, MspJI, endonuclease, exonuclease, etc.). The cleavable moiety may comprise an abasic site or an analog of an abasic site (e.g., dSpacer), a dideoxyribose, a spacer, e.g., C3 spacer, hexanediol, triethylene glycol spacer (e.g., Spacer 9), hexa-ethyleneglycol spacer (e.g., Spacer 18), a photocleavable moiety, or combinations or analogs thereof. Alternatively, or in addition, the cleavable moiety may be cleavable using one or more stimuli, e.g., photo-stimulus, chemical stimulus, thermal stimulus, etc.

The sequencing workflow 100 may not involve supports, for example when a template nucleic acid and/or its derivatives are directly attached to a substrate and amplified and/or sequenced from the substrate.

A template nucleic acid may include an insert sequence sourced from a biological sample. The template nucleic acid may be derived from any nucleic acid of the biological sample (e.g., endogenous nucleic acid) and result from any number of processing operations, such as but not limited to fragmentation, degradation or digestion, transposition, ligation, reverse transcription, extension, replication, etc. The template nucleic acid may be single-stranded, double-stranded, or partially double-stranded. A template nucleic acid may comprise one or more functional nucleic acid sequences. For example, the template nucleic acid may comprise a capture sequence, a primer sequence, a sequencing primer sequence, a barcode sequence, a sample index sequence, a unique molecular identifier (UMI), a flow cell adaptor sequence, an adaptor sequence, a target sequence, a random sequence, a binding sequence (e.g., for a splint, primer, template nucleic acid, capture sequence, etc.), or any other functional sequence useful for a downstream operation, a complement thereof, or any combination thereof. The template nucleic acid may comprise an adaptor sequence configured to be captured by a capture sequence of an oligonucleotide coupled to a support. The one or more functional nucleic acid sequences may be disposed at one end or both ends of the insert sequence. A nucleic acid molecule comprising the insert sequence, or complement thereof, may be processed with (e.g., attached to, extend from, etc.) one or more adaptor molecules to generate the template nucleic acid comprising the insert sequence and one or more functional nucleic acid sequences. A template nucleic acid may comprise one or more capture entities and/or one or more cleavable moieties that are described elsewhere herein.

Optionally, the supports and/or template nucleic acids may be pre-enriched (102). For example, a support comprising a distinct oligonucleotide sequence is pre-enriched to isolate from a mixture comprising support(s) that do not have the distinct oligonucleotide sequence. For example, a template nucleic acid comprising a distinct configuration (e.g., comprising a particular adaptor sequence) is pre-enriched to isolate from a mixture comprising template nucleic acids that do not have the distinct configuration. In some cases, the capture entity on the supports and/or template nucleic acids are used for pre-enrichment.

The supports and template nucleic acids may be attached (103) to generate support-template complexes. A template nucleic acid may be coupled to a support via any method(s) that results in a stable association between the template nucleic acid and the support. For example, the template nucleic acid may hybridize to an oligonucleotide on the support; the template nucleic acid may be ligated to a nucleic acid coupled to the support; the template nucleic acid may hybridize to one or more intermediary molecules, such as a splint, bridge, and/or primer molecule, which hybridizes to an oligonucleotide on the support; and/or the template nucleic acid may be hybridized to an oligonucleotide on a support, which oligonucleotide comprises a primer sequence which is extended. In some cases, the respective concentrations of the supports and template nucleic acids may be adjusted such that a majority of support-template complexes are single template-attached supports (e.g., a support attached to a single template nucleic acid).

Optionally, support-template complexes may be pre-enriched (104), wherein a support-template complex is isolated from a mixture comprising support(s) and/or template nucleic acid(s) that are not attached to each other. In some cases, the capture entity on the supports and/or template nucleic acids are used for pre-enrichment.

The template nucleic acids may be subjected to amplification reactions (105) to generate a plurality of amplification products immobilized to the support. Such amplification reactions may comprise performing polymerase chain reaction (PCR) or any other amplification methods described herein, including but not limited to emulsion PCR (ePCR or emPCR), isothermal amplification, recombinase polymerase amplification (RPA), rolling circle amplification (RCA), multiple displacement amplification (MDA), bridge amplification, template walking, etc. Amplification reactions can occur while the support is immobilized to a substrate. Amplification reactions can occur off the substrate, such as in solution, or on a different surface or platform. Amplification reactions can occur in isolated reaction volumes, such as within multiple droplets in an emulsion during emulsion PCR (ePCR or emPCR), or in wells or tubes.

Optionally, subsequent to amplification, the supports, template nucleic acids, and/or support-template complexes may be subjected to post-amplification processing (106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described in U.S. Pat. Nos. 10,900,078, U.S. Patent Pub. No. 20210079464A1, and International Patent Pub. No. WO2022040557A2, each of which is entirely incorporated by reference herein.

The template nucleic acids may be subject to sequencing (107). The template nucleic acid(s) may be sequenced while attached to the support. Alternatively, the template nucleic acid molecules may be free of the support when sequenced and/or analyzed. The template nucleic acids may be sequenced while immobilized to a substrate, such as via a support or otherwise. Examples of substrate-based sample processing systems are described elsewhere herein. Any sequencing method may be used, for example pyrosequencing, single molecule sequencing, sequencing by synthesis (SBS), sequencing by ligation, sequencing by binding, etc.

For example, sequencing comprises extending a sequencing primer (or growing strand) hybridized to a template nucleic acid by providing labeled nucleotide reagents, washing away unincorporated nucleotides from the reaction space, and detecting one or more signals from the labeled nucleotide reagents which are indicative of an incorporation event or lack thereof. After detection, the labels may be cleaved and the whole process may be repeated any number of times to determine sequence information of the template nucleic acid. One or more intermediary flows may be provided intra- or inter-repeat, such as washing flows, label cleaving flows, terminator cleaving flows, reaction-completing flows (e.g., double tap flow, triple tap flow, etc.), labeled flows (or bright flows), unlabeled flows (or dark flows), phasing flows, chemical scar capping flows, etc. A nucleotide mixture that is provided during any one flow may comprise only labeled nucleotides, only unlabeled nucleotides, or a mixture of labeled and unlabeled nucleotides. The mixture of labeled and unlabeled nucleotides may be of any fraction of labeled nucleotides, such as at least or at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A nucleotide mixture that is provided during any one flow may comprise only non-terminated nucleotides, only terminated nucleotides, or a mixture of terminated and non-terminated nucleotides. When using only non-terminated nucleotides, terminator cleaving flows may be omitted from the sequencing process. When using terminated nucleotides, to proceed with the next step of extension, prior to, during, or subsequent to detection, a terminator cleaving flow may be provided to cleave blocking moieties. A nucleotide mixture that is provided during any one flow may comprise any number of canonical base types (e.g., A, T, G, C, U), such as a single canonical base type, two canonical base types, three canonical base types, four canonical base types or five canonical base types (including T and U). Different types of nucleotide bases may be flowed in any order and/or in any mixture of base types that is useful for sequencing. Various flow-based sequencing systems and methods are described in U.S. Pat. Pub. No. 2022/0170089A1, which is entirely incorporated by reference herein for all purposes and described further below. Labeled nucleotides may comprise a dye, fluorophore, or quantum dot, multiples thereof, and/or combination thereof. In some cases, nucleotides of different canonical base types may be labeled and detectable at a single frequency (e.g., using the same or different dyes). In other cases, nucleotides of different canonical base types may be labeled and detectable at different frequencies (e.g., using the same or different dyes).

Subsequent to sequencing, the sequencing signals collected and/or generated may be subjected to data analysis (108). The sequencing signals may be processed to generate base calls and/or sequencing reads. In some cases, the sequencing reads may be processed to generate diagnostics data to the biological sample, or the subject from which the biological sample was derived from. The data analysis may comprise image processing, alignment to a genome or reference genome, training and/or trained algorithms, error correction, and the like.

While the sequencing workflow 100 with respect to FIG. 1 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

It will be appreciated that in some instances, the different operations described in the sequencing workflow 100 may be performed in a different order. It will be appreciated that in some instances, one or more operations described in the sequencing workflow 100 may be omitted or replaced with other comparable operation(s). It will be appreciated that in some instances, one or more additional operations described in the sequencing workflow 100 may be performed. The different operations described with respect to sequencing workflow 100 may be performed with the help of open substrate systems described herein.

### Sequencing Methods

### Non-Terminated Sequencing, Flow-Based Sequencing

Sequencing data can be generated using flow-based sequencing methods that include extending a primer bound to a template nucleic acid according to a pre-determined flow cycle and/or flow order where, in one or more flow positions, known canonical base type(s) of nucleotides (e.g., A, C, G, T, U) is accessible to the extending primer. At least some of the nucleotides may include a label, which labeled nucleotides upon incorporation into the extending primer render a detectable signal. The resulting sequence by which nucleotides are incorporated into the extended primer is expected to be the reverse complement of the sequence of the template nucleic acid. A method for sequencing can comprise using a flow sequencing method that includes (1) extending a primer using labeled nucleotides in a flow, and (2) detecting the presence or absence of a labeled nucleotide incorporated into the extending primer to generate sequencing data. Flow sequencing methods may also be referred to as “natural sequencing-by-synthesis,” “mostly natural sequencing-by-synthesis,” or “non-terminated sequencing-by-synthesis” methods. Example methods are described in U.S. Pat. No. 8,772,473 and U.S. Pat. Pub. No. 2022/0170089A1, each of which is incorporated herein by reference in its entirety.

In terminated sequencing methods, a bright step may comprise terminated nucleotides (e.g., reversibly terminated nucleotides). In some cases, a bright step may comprise a single nucleotide base type (e.g., A, C, G, T, U) or a mixture of nucleotide base types (e.g., 2, 3, 4, etc.) and, at most, a single nucleotide base may be incorporated into a growing strand. In a flow sequencing extension step comprising a mixture of reversibly terminated and unterminated nucleotides, more than one nucleotide base may be incorporated into a growing strand, the last incorporation being of a terminated nucleotide.

In flow sequencing, iterative nucleotide flows are used to extend the primer hybridized to the template nucleic acid, with detection of incorporated nucleotides between one or more flows. The nucleotides may be, for example, non-terminating nucleotides such that more than one consecutive base can be incorporated into the extending primer strand if more than one consecutive complementary base (or homopolymer region) is present in the template strand. At least a portion of the nucleotides can be labeled so that incorporation can be detected. Generally, only a single nucleotide type is introduced in a flow, although two or three different types of nucleotides may be simultaneously introduced in certain embodiments. This methodology can be contrasted with sequencing methods that use a reversible terminator, where primer extension is stopped after extension of every single base before the terminator is reversed (e.g., by removing a 3′ blocking group) to allow incorporation of the next succeeding base.

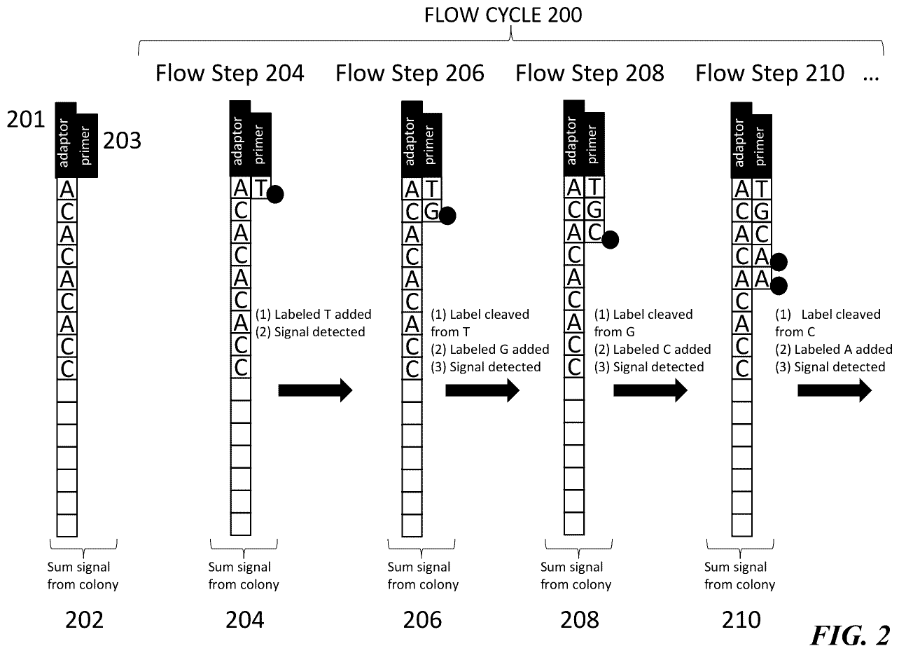

FIG. 2 illustrates an example flow sequencing method that can be used to generate the sequencing data described herein. Template nucleic acids may be immobilized to a surface (e.g., the surface of a bead attached to a substrate or directly to a substrate), as described in detail herein. In this example, the template nucleic acid includes an adaptor sequence 201 followed by an insert sequence (“ACGTTGCTA . . . ”). The adaptor sequence 201 can include a sequencing primer hybridization site. At operation 202, a sequencing primer 203 is hybridized to the adaptor sequence 201 at the sequencing primer hybridization site. The sequencing primer 203 is then extended in a series of flows according to flow cycle 200 with flow order: [T G C A]. In this example, the flow cycle 200 includes four flow steps 204, 206, 208, 210, and in a given flow step, a single base type is provided to the template-primer hybrid. In flow step 204, nucleotides comprising labeled T nucleotides are provided; in flow step 206, nucleotides comprising labeled G nucleotides are provided; in flow step 208, nucleotides comprising labeled C nucleotides are provided; in flow step 210, nucleotides comprising labeled A nucleotides are provided. Nucleotides in a single-base flow may comprise a mixture of labeled and unlabeled nucleotides of the single base. These nucleotides may be unterminated. At flow step 204, a labeled T nucleotide is incorporated by the extending sequencing primer 203 opposite the A base in the template strand. Then, a signal indicative of the incorporation of the labeled T nucleotide can be detected. For example, the signal may be detected by imaging the surface the template nucleic acids are immobilized on and analyzing the resulting image(s). The sequencing platform may be washed with a wash buffer to remove unincorporated nucleotides prior to signal detection. In some cases, prior to the next flow step (e.g., 206), the label may be removed from the incorporated labeled T nucleotide (e.g., by cleaving the label from the nucleotide), before proceeding. Nucleotide flow, detection, and optionally cleavage, may be repeated according to a flow order that may or may not include repeating the flow cycle 200 for any number of times. Flow step 210 illustrates incorporation of two labeled A bases by the extending sequencing primer 203 opposite the two T bases in the template strand, per the non-terminated nature of the flown nucleotides. The detected signal intensity indicating the incorporation of two A nucleotides may be greater than the signal intensity indicating the incorporation of one nucleotide. For simplicity, this Figure illustrates incorporation of two labeled A nucleotides in the same hybrid. However, flow-based sequencing may be performed on colonies of amplified molecules, e.g., each bead representing one colony, where an optically resolvable location contains multiple copies of the same template nucleic acid molecule (e.g., a location contains one amplified bead), such that the signal detected at an optically resolvable location represents an aggregate signal from the multiple copies of molecules. Thus, when using a nucleotide flow mixture containing labeled and unlabeled nucleotides of a same base type, the incorporation of the labeled nucleotides can be distributed across the multiple copies of the molecules, and the aggregate signal from the multiple copies detected. In some cases, for a majority of hybrids, at most a single labeled nucleotide may be incorporated into a single homopolymer stretch in a hybrid—the longer the homopolymer stretch, the more likely that more hybrids of the plurality of copies of hybrids in an optically resolvable location will incorporate one labeled nucleotide.

While each flow step in the example flow sequencing method in FIG. 2 results in incorporation of one or more nucleotides (and thus a detected signal indicating such incorporation), it should be appreciated that not all flow steps result in incorporation of nucleotides. In some flow steps, no nucleotide base may be incorporated (for example, in the absence of a complementary base in the template).

The flow signal (i.e., the number of incorporated nucleotides) for any flow position in a sequencing data set is flow order dependent in that the flow order used to sequence the nucleic acid molecule at any base position can affect the flow signal at that position. As further described herein, this discovery can be taken advantage of in one or more manners. For example, a given nucleic acid molecule or a region of nucleic acid molecule may be sequenced (with either dark or light flows) using a first flow order, and re-sequenced using a second (different) flow order, thus providing a different flow sequence context across the nucleic acid molecule. If the likelihood match of the nucleic acid molecule with a variant to a candidate sequence with the variant is low using one flow order, the likelihood match of the nucleic acid molecule to the candidate sequence may be high using the second flow order. In some cases, the flow order can be an extended flow cycle (e.g., with more than four base types in a cycle), meaning that it is not simply a four-flow periodic repeat of the four base types A, C, T and G. In some cases, the repeating unit is longer than four bases, such as a pattern comprising all possible two-base flow sequences (i.e., all X-Y pairs are within the repeating unit where X is all four bases and Y is each of the non-X bases) or three-base flow sequences (i.e., all possible X-Y-Z permutations are within the repeating unit). In some cases, a flow sequencing order may be selected to target a specific genetic variant (e.g., in targeted sequencing when known haplotypes may be present and/or when known haplotypes can result in differing flow signals at one or more flow positions).

Sequencing flow orders of unterminated nucleotides may have different efficiencies. (i.e., the average number of nucleotide incorporations per flow when used to sequence a human reference genome). In some embodiments, the flow order has an efficiency of about 0.6 or greater (such as about 0.62 or greater, about 0.64 or greater, about 0.65 or greater, about 0.66 or greater, or about 0.67 or greater). In some embodiments, the flow order has an efficiency of about 0.6 to about 0.7.

A nucleotide mixture that is provided during any one flow may comprise only labeled nucleotides, only unlabeled nucleotides, or a mixture of labeled and unlabeled nucleotides. The mixture of labeled and unlabeled nucleotides may be of any fraction of labeled nucleotides, such as at least or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. Labeled nucleotides may comprise a dye, fluorophore, or quantum dot, multiples thereof, and/or combination thereof. In some cases, nucleotides of different canonical base types may be labeled and detectable at a single frequency (e.g., using the same or different dyes). In other cases, nucleotides of different canonical base types may be labeled and detectable at different frequencies (e.g., using the same or different dyes). Labeled nucleotides may comprise an optical moiety (e.g., dye, fluorophore, quantum dot, label, etc.) coupled to a nucleobase via a linker, and the label from the labeled nucleotides may be removed by cleaving the linker to remove the optical moiety. Cleaving may comprise one or more stimuli, such as exposure to a chemical (e.g., reducing agent), an enzyme, light (e.g., UV light), or temperature change (e.g., heat).

Flow-based sequencing may comprise providing non-detected nucleotide flow(s), for example to skip sequencing of a region(s) of the template nucleic acid; to ensure completion of incorporation reactions across all template-primer hybrids in the reaction space; and/or phasing or re-phasing. A non-detected nucleotide flow may be referred to herein as a “dark flow”, “dark tap”, or “dark tap flow.” A detected nucleotide flow may be referred to herein as a “bright flow”, “bright tap”, or “bright tap flow.” Incorporation reactions may be incomplete in the reaction space when not all available incorporation sites in the template-primer hybrids have incorporated a complementary base, such as due to reaction kinetics and/or insufficient incubation time or reagents. In some cases, single-base flows of the same canonical base type may be provided consecutively (without intervening flow of a different nucleotide base type) for any number of consecutive flows, to ensure completion of incorporation reactions. A consecutive same-base flow may be referred to herein as a “double tap” or “double tap flow” if there are two consecutive flows, a “triple tap” or “triple tap flow” if there are three consecutive flows, or a “nth tap” or “nth tap flow” if there are n consecutive flows of the same base type. A double tap, triple tap, or nth tap flow may or may not be detected. Labels in a flow may or may not be removed (e.g., cleaved) prior to the double tap, triple tap, or nth tap flow. Detection of labeled nucleotides from a particular flow may be performed prior to, during, or subsequent to the double tap, triple tap, or nth tap flow. Accordingly, below are non-limiting examples of flow cycles that can be used in a larger flow order of flow-based sequencing methods, which may or may not be repeated and/or mixed and matched with other flow cycles, where * after a base represents a detected flow step and/between bases represents a mixed base flow:
Single-base flow: e.g., [T*A*C*G*]
Single-base flow with double tap: e.g., [T*T A*A C*C G*G]
Mixed base flow, all labeled: e.g., [T*A*/C*/G*]
Mixed base flow, some unlabeled: e.g., [T*A/C*/G]
Mixed base flow, some unlabeled: e.g., [T A*/C*/G*]
Skip region base flow: e.g., [T/A/C G/A/T]
Three base flow cycle: e.g., [T A C]

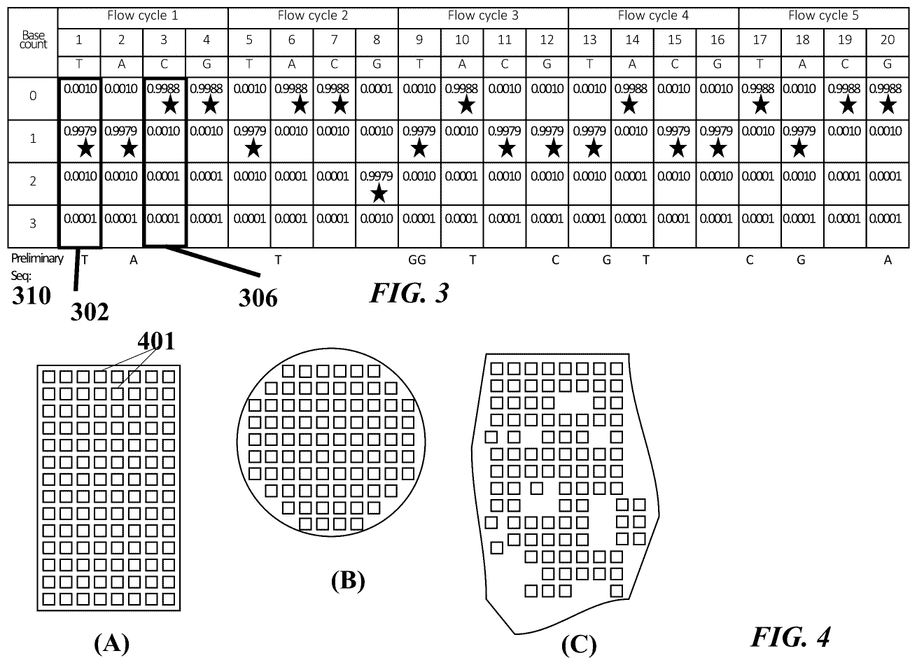

FIG. 3 illustrates an example flowgram of signals detected after five exemplary flow cycles of [T A C G] are performed to extend a sequencing primer, in accordance with some cases. Each column in the flowgram corresponds to a detected flow step (e.g., 302, 306), and the values in each column collectively represent the detected signal intensity in the flow step. In each detected flow step, the flow signal can be determined from an analog signal that is detected during the sequencing process, such as a fluorescent signal of the one or more bases incorporated. In some cases, for a flow step, the detected signal intensity can be expressed in probabilistic terms. Specifically, the detected signal intensity can be expressed in a series of likelihood values corresponding to different integer homopolymer base lengths (e.g., 0 base, 1 base, 2 bases, 3 bases, etc.) for the flow position. For flow step 302, the detected signal intensity is expressed by a first likelihood value of 0.001 for 0 base, a second likelihood value of 0.9979 for 1 base, a third likelihood value of 0.001 for 3 bases, and a fourth likelihood value of 0.0001 for 4 bases. This can be interpreted to indicate that there is a high statistical likelihood that one nucleotide base has been incorporated. In this flow step, a single T was determined to be incorporated, which means there is an A in the template. Similarly, for flow step 306, the column values can collectively indicate that there is a high statistical likelihood that no base has been incorporated (with 0.9988 likelihood value for 0 bases). With similar analyses performed at each flow position, a preliminary sequence 310 (TATGGTCGTCGA) of the extending primer can be determined, and reverse complement (i.e., the template strand sequence) readily determined from the preliminary sequence. For example, the most likely sequence can be determined by selecting the base count with the highest likelihood at each flow position, as shown by the stars in the flowgram. Further, the likelihood of this sequencing data set can be determined as the product of the selected likelihood at each flow position. Accordingly, the flowgram may be formatted as a sparse matrix, with a flow signal represented by a plurality of likelihood values indicative of a plurality of base homopolymer length counts at each flow position. The homopolymer length likelihood may vary, for example, based on the noise or other artifacts present during detection of the analog signal during sequencing. In some cases, if the homopolymer length likelihood statistical parameter or likelihood is below a predetermined threshold, the parameter may be set to a predetermined non-zero value that is substantially zero (i.e., some very small value or negligible value) to aid the downstream statistical analysis further discussed herein, wherein a true zero value may give rise to a computational error or insufficiently differentiate between levels of unlikelihood, e.g., very unlikely (0.0001) and inconceivable (0). Thus, a method for sequencing may comprise generating a flowgram using analog signals (e.g., fluorescent signals) detected from a template nucleic acid or derivative thereof and generating base calls and/or sequencing reads using the flowgram.

As will be appreciated, in flow-based sequencing, the signal for any flow position in the sequencing data is flow order-dependent in that the same flow position for a same template nucleic acid may express different flow signals for different flow orders. Any useful predetermined flow cycles and/or flow orders may be designed to sequence a template nucleic acid and/or more accurately or precisely detect a particular type of sequence (e.g., single nucleotide polymorphisms (SNPs)) within the template nucleic acid (e.g., of a genome).

A flowgram may be binary or non-binary. A binary flowgram detects the presence (1) or absence (0) of an incorporated nucleotide. A non-binary flowgram, such as shown in FIG. 3, can more quantitatively determine a number of incorporated nucleotides at each flow position.

During sequencing by synthesis, a sequencing primer may be hybridized to a template (e.g., to a primer binding site on the template) and extended in a stepwise manner by, in each step, contacting the hybrid with nucleotide reagents of known canonical base type(s). The extended or extending sequencing primer may also be referred to herein as a growing strand. An extension step may be a bright step (also referred to herein, in some cases, as labeled step or detected step) or a dark step (also referred to herein, in some cases, as an unlabeled step or undetected step). A sequencing method may comprise only bright steps. Alternatively, a sequencing method may comprise a mix of bright step(s) and dark step(s). For a bright step, the growing strand may be contacted with nucleotide reagents that include labeled nucleotides (of known canonical base type(s)) and signals indicative of incorporation of the labeled nucleotides, or lack thereof, may be detected to determine a base or sequence of the template. Alternatively or in addition, for a bright step, the growing strand may be contacted with a mixture of labeled and unlabeled nucleotide reagents. For a dark step, the growing strand may be contacted with solely unlabeled nucleotide reagents. Alternatively or in addition, for a dark step, the growing strand may be contacted with labeled nucleotide reagents and detection omitted.

Flow-based sequencing methods and non-terminated sequencing-by-synthesis methods have been generally described elsewhere herein. In terminated sequencing-by-synthesis methods, a bright step may comprise terminated nucleotides (e.g., reversibly terminated nucleotides). In some cases, a bright step may comprise a mixture of nucleotide base types (e.g., 2, 3, 4, or more base types). A dark step may comprise terminated nucleotides, unterminated nucleotides, or a mixture thereof. A dark step may comprise a single nucleotide base type. Alternatively, a dark step may comprise a mixture of nucleotide base types. In an extension step comprising solely reversibly terminated nucleotides (e.g., and not unterminated nucleotides) a single nucleotide base may be incorporated into a growing strand. In an extension step comprising a mixture of reversibly terminated and unterminated nucleotides, more than one nucleotide base may be incorporated into a growing strand.

### Sequencing Systems

The sequencing methods described herein may be performed using any sequencing platform, such as a substrate-based system. The substrate-based system may comprise a closed substrate such as a flow cell comprising one or more fluidic or microfluidic channels, wells, and/or microwells. For example, template nucleic acids on or off a bead may be immobilized to a surface in a flow cell, and reagents flowed in and out of the flow cell through channels in the flow cell to contact the template nucleic acids. The channels may be flushed with wash buffers between different reagent cycles. The substrate-based system may comprise an open substrate. For example, template nucleic acids on or off a bead may be immobilized to a surface of an open substrate, and reagents directed to the surface, such as via nozzles (e.g., across an air gap), to contact the template nucleic acids. The open substrate may be washed with wash buffers between different reagent cycles.

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as increased efficiency achieved from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of analytes and reagents onto the substrate. The sample processing may permit highly efficient imaging of one or more analytes, or signals corresponding thereto, on the substrate. The sample processing system may comprise an imaging system comprising a detector. Substrates, detectors, and sample processing hardware that can be used in the sample processing system are described in further detail in U.S. Patent Pub. No. 20200326327A1, U.S. Patent Pub. No. 20210079464A1, International Patent Pub. No. WO2022072652A1, U.S. Patent Pub. No. 20210354126A1, and International Patent Pub. No. WO2023192403A2, each of which is entirely incorporated herein by reference for all purposes.

A substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). A surface of the substrate may be textured or patterned. For example, the substrate may comprise grooves, troughs, hills, pillars, wells, cavities (e.g., micro-scale cavities or nano-scale cavities), channels, wedges, cuboids, cylinders, spheroids, hemispheres, etc. A substrate surface may comprise chemical groups such as amines, esters, hydroxyls, epoxides, and the like, or a combination thereof. A substrate surface may comprise any of the binders or linkers described herein, such as to help immobilize analytes thereto. The substrate may be textured or patterned such that all features are at or above a reference level of the surface (no features below a reference level of the surface, such as a well), or such that all features are at or below a reference level of the surface (no features below a reference level of the surface, such as a pillar). In some instances, a texture of the substrate may comprise structures having a maximum dimension of at most about 500%, 400%, 300%, 200%, 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. Alternatively, the substrate may be untextured and/or unpatterned.

The substrate may have the general form of a cylinder, a cylindrical shell or disk, a rectangular prism, or any other geometric form. The substrate may have a thickness (e.g., a minimum dimension) of at least and/or at most about 100 micrometers (μm), 200 μm, 500 μm, 1 millimeter (mm), 2 mm, 5 mm, 10 mm, 15 mm, 20 mm, 25 mm, 30 mm, 35 mm, 40 mm, 45 mm, 50 or mm. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least and/or at most about 1 mm, 2 mm, 5 mm, 10 mm, 20 mm, 30 mm, 40 mm, 50 mm, 100 mm, 150 mm, 200 mm, 300 mm, 400 mm, 500 mm, 1,000 mm, 1,500 mm, 2,000 mm, 2,500 mm, 3,000 mm, 4,000 mm, 5,000 mm or more.

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing. In some cases, the individually addressable locations may be defined by physical features of the substrate (e.g., on a modified surface) to distinguish them from each other and from non-individually addressable locations. In some cases, the individually addressable locations may not be defined by physical features of the substrate, and instead may be defined digitally (e.g., by indexing) and/or via the analytes and/or reagents that are loaded on the substrate (e.g., the locations in which analytes are immobilized on the substrate). The plurality of individually addressable locations may be arranged as an array, randomly, or according to any pattern, on the substrate. FIG. 4 illustrates different substrates (from a top view) comprising different arrangements of individually addressable locations 401, with panel A showing a substantially rectangular substrate with regular linear arrays, panel B showing a substantially circular substrate with regular linear arrays, and panel C showing an arbitrarily shaped substrate with irregular arrays.

The substrate may have any number of individually addressable locations, for example, on the order of 1, 101, 102, 103, 104, 105, 106, 107, 108, 109, 1010, 1011, 1012, 1013 or more individually addressable locations. Each individually addressable location may have any shape or form, for example the general shape or form of a circle, oval, square, rectangle, polygonal, or non-polygonal shape when viewed from the top. A plurality of individually addressable locations can have uniform shape or form, or different shapes or forms. An individually addressable location may have any size. In some cases, an individually addressable location may have an area of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5, 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 7, 8, 9, 10 square micron (μm²), or more. The individually addressable locations may be distributed on a substrate with a pitch determined by the distance between the center of a first location and the center of the closest or neighboring individually addressable location. Locations may be spaced with a pitch of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5, 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, or 10 micron (μm). In some cases, the pitch between two individually addressable locations may be determined as a function of a size of a loading object (e.g., bead). For example, where the loading object is a bead having a maximum diameter, the pitch may be at least about the maximum diameter of the loading object.

An individually addressable location may be capable of immobilizing thereto an analyte (e.g., a nucleic acid, a protein, a carbohydrate, etc.) or a reagent (e.g., a nucleic acid, a probe molecule, a barcode molecule, an antibody molecule, a primer molecule, a bead, etc.). In some cases, an analyte or reagent may be immobilized to an individually addressable location via a support, such as a bead. In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location. A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.). In some cases, an individually addressable location may comprise a distinct surface chemistry. The distinct surface chemistry may distinguish between different addressable locations and/or distinguish an individually addressable location from surrounding locations. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location.

A substrate may comprise a binder or linker configured to immobilize an analyte or reagent to an individually addressable location. The binders may be integral to or added to the substrate. The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions, such as hybridization between two nucleic acid molecules (an oligonucleotide binder and a template nucleic acid). For example, the binders may comprise one or more of antibodies, oligonucleotides, nucleic acid molecules, aptamers, affinity binding proteins, lipids, carbohydrates, and the like.

The substrate may be rotatable about an axis, referred to herein as a rotational axis. The rotational axis may or may not be an axis through the center of the substrate. The systems, devices, and apparatus described herein may further comprise an automated or manual rotational unit configured to rotate the substrate. The rotational unit may comprise a motor and/or a rotor. For instance, the substrate may be affixed to a chuck (such as a vacuum chuck). The substrate may be rotated at a rotational speed of at least about 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with different rotational velocities during different operations described herein, for example with higher velocities during reagent dispense and with lower velocities during analyte loading and imaging operations. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For example, bead loading may be performed with controlled dispensing. For controlled dispensing, the substrate may rotate with a rotational frequency of no more than 60, 50, 40, 30, 25, 20, 15, 14, 13, 12, 11, 10, 9, 8, 7, 6, 5, 4, 3, 2, 1 rpm or less. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, low speed for analyte or reagent incubation, etc.).

In some cases, the substrate may be movable in any vector or direction. For example, such motion may be non-linear (e.g., in rotation about an axis), linear (e.g., on a rail track), or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

Reagents and/or analytes may be delivered to the surface of the substrate using one or more fluid nozzles. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles. In some cases, the reagents and/or analytes are delivered across a non-solid gap, such as an air gap. There may be any number of dispensing nozzles, such as 1, 2, 3, 4, 5, 6, 7, 8, 9, 10, 11, 12, 13, 14, 15, 16, 17, 18, 19, 20 or more dispensing nozzles. In some cases, different reagents (e.g., nucleotide solutions of different types, different probes, washing solutions, etc.) may be dispensed via different nozzles, such as to prevent contamination where each nozzle may be connected to a dedicated fluidic line or fluidic valve, which may further prevent contamination. Alternatively, some nozzles may share a fluidic line or fluidic valve, such as for pre-dispense mixing and/or to dispensing to multiple locations.

In some cases, a solution may be dispensed on the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate. The substrate may then be subjected to vibration, which may spread the reagent to different locations across the substrate. Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

A sample may comprise beads, as described elsewhere herein, for example beads comprising nucleic acid colonies bound thereto. In some cases, an order of magnitude of at least and/or at most about 101, 102, 103, 104, 105, 106, 107, 108, 109, 1010, 1011, 1012, 1013 or more beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag (e.g., barcode) that identifies a bead amongst a plurality of beads. FIG. 7 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface.

Dispense mechanisms described herein may be operated by a fluid flow unit which may be controlled by one or more controllers, individually or collectively. The fluid flow unit may comprise any of the hardware and software components described with respect to the dispense mechanisms herein.

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A signal may be an optical signal (e.g., fluorescent signal), electronic signal, or any detectable signal. The signal may be detected during rotation of the substrate or following termination of the rotation. The signal may be detected while the analyte is in fluid contact with a solution. The signal may be detected following washing of the solution. In some instances, after the detection, the signal may be muted, such as by cleaving a label from a probe and/or the analyte, and/or modifying the probe and/or the analyte. Such cleaving and/or modification may be performed by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times by the system. Such operations may be repeated in an iterative manner. For example, the same analyte immobilized to a given location in the array may interact with multiple solutions in multiple cycles and for each iteration, the additional signals detected may provide incremental, or final, data about the analyte during the processing. For example, when sequencing a nucleic acid molecule, additional signals detected for each iteration may be indicative of one or more bases in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly, electronically or computationally, advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar function by high-speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, tube lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors, CCD cameras, CMOS cameras, TDI cameras (e.g., TDI line-scan cameras), pseudo-TDI rapid frame rate sensors, or CMOS TDI or hybrid cameras. The optical system may further comprise any one or more optical sources (e.g., lasers, LED light sources, etc.). In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g., interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise one or more controllers operatively coupled to the one or more sensors, individually or collectively programmed to process optical signals from the one or more sensors, such as for each region of the rotating substrate.

In some cases, the optical system may comprise an immersion objective lens. The immersion objective lens may be in contact with an immersion fluid that is in contact with the open substrate. The immersion fluid may comprise any suitable immersion medium for imaging (e.g., water, aqueous, organic solution). In some cases, an enclosure may partially or completely surround a sample-facing end of the optical imaging objective. The enclosure may be configured to contain the immersion fluid. The enclosure may not be in contact with the substrate; for example, a gap between the enclosure and the substrate may be filled by the fluid contained by the enclosure (e.g., the enclosure can retain the fluid via surface tension). In some cases, an electric field may be used to regulate a hydrophobicity of one or more surfaces of the container to retain at least a portion of the fluid contacting the immersion objective lens and the open substrate. In some cases, the immersion fluid may be continuously replenished or recycled via an inlet and outlet to the enclosure.

One or more surfaces of the substrate may be exposed to and accessible from a surrounding open environment. In some cases, the surrounding open environment may be controlled and/or confined in a larger controlled environment. An open substrate may be processed within a modular local sample processing environment. A barrier comprising a fluid barrier may be maintained between a sample processing environment and an exterior environment during certain processing operations, such as reagent dispensing and detecting. Systems and methods comprising a fluid barrier are described in further detail in U.S. Patent Pub. No. 20210354126A1, which is entirely incorporated herein by reference. A modular local sample processing environment may be defined by a chamber and a lid plate, where the lid plate is not in contact with the chamber, and the gap between the lid plate and the chamber may comprise the fluid barrier. The fluid barrier may comprise fluid (e.g., air) from the sample processing environment and/or the exterior environment and may have lower pressure than the sample processing environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least and/or at most 20 degrees Celsius (° C.), 25° C., 30° C., 35° C., 40° C., 45° C., 50° C., 55° C., 60° C., 65° C., 70° C., 75° C., 80° C., 85° C., 90° C., 95° C., 100° C., or more. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

An open substrate may be retained in the same or approximately the same physical location during processing of an analyte and subsequent detection of a signal associated with the processed analyte. Alternatively, different operations on or with the open substrate may be performed in different stations disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. The open substrate may transition between different stations by transporting the sample processing environment comprising the chamber containing the open substrate between the different stations. One or more mechanical components or mechanisms, such as a robotic arm, elevator mechanism, actuators, rails, and the like, or other mechanisms may be used to transport the sample processing environment.

One or more environmental units (e.g., humidifiers, heaters, heat exchangers, compressors, etc.) may be configured to, individually or collectively, regulate one or more operating conditions in one or more stations. In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

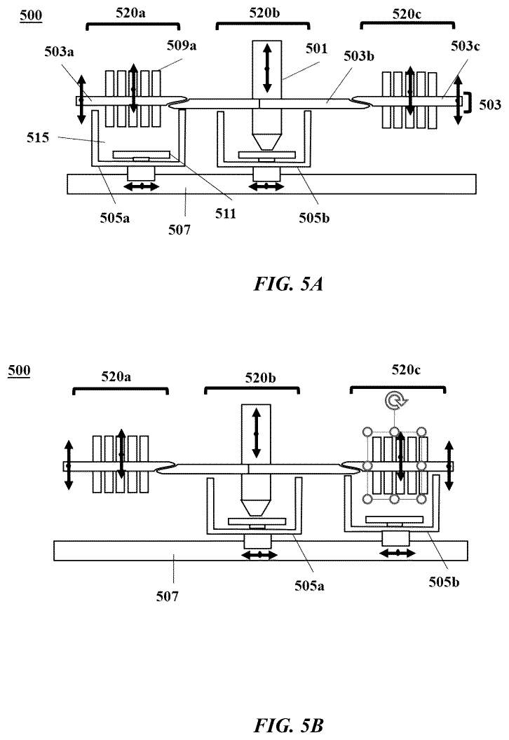

One or more modular sample environment systems (each having its own barrier system, e.g., fluid barrier) can be used between the different stations. In some instances, the systems described herein may be scaled up to include two or more of a same station type. For example, a sequencing system may include multiple processing and/or detection stations. FIGS. 5A-5B illustrate a system 500 that multiplexes two modular sample environment systems in a three-station system. In FIG. 5A, a first chemistry station (e.g., 520a) can operate (e.g., dispense reagents, e.g., to incorporate nucleotides to perform sequencing by synthesis) via at least a first operating unit (e.g., fluid dispenser 509a) on a first substrate (e.g., 511) in a first sample environment system (e.g., 505a) while substantially simultaneously, a detection station (e.g., 520b) can operate (e.g., scan) on a second substrate in a second sample environment system (e.g., 505b) via at least a second operating unit (e.g., detector 501), while substantially simultaneously, a second chemistry station (e.g., 520c) sits idle. An idle station may not operate on a substrate. An idle station (e.g., 520c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 5B, where the second substrate in the second sample environment system (e.g., 505b) is re-stationed from the detection station (e.g., 520b) to the second chemistry station (e.g., 520c) for operation (e.g., dispensing of reagents, e.g., to incorporate nucleotides to perform sequencing by synthesis) by the second chemistry station, and the first substrate in the first sample environment system (e.g., 505a) is re-stationed from the first chemistry station (e.g., 520a) to the detection station (e.g., 520b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 507) to the different stations and/or the different stations may be physically moved to the different sample environment systems. One or more components of a station, such as modular plates 503a, 503b, 503c of plate 503 (e.g., lid plate) defining a particular station(s), may be physically moved to allow a sample environment system to exit the station, enter the station, or cross through the station. During processing of a substrate at station, the environment of a sample environment region (e.g., 515) of a sample environment system (e.g., 505a) may be controlled and/or regulated according to the station's requirements. After the next operating cycle is complete, the sample environment systems can be re-stationed again, such as back to the configuration of FIG. 5A, and this re-stationing can be repeated (e.g., between the configurations of FIGS. 5A and 5B) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations substantially simultaneously while the detection station is kept idle.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

### Compiling Sequence Reads Across Multiple Sequencing Runs

Provided herein are systems, methods, compositions, and kits for improving the accuracy of sequencing reads. In some embodiments, this is achieved by reading a homopolymer section of a template in multiple shorter segments. In some embodiments, this is achieved by reading a homopolymer section using multiple labels simultaneously. Additionally, provided herein are systems, methods, compositions, and kits for improving the speed and accuracy of sequencing with a combination of reversibly terminated and unterminated sequences. Such systems, methods, compositions, and kits can be applied alternatively or in addition to the sequencing 107 operation described with respect to sequencing workflow 100 of FIG. 1 and, optionally, in the absence of pre-enrichment 102, 103, amplification of templates 105, and/or post-amplification processing 106. Such devices, systems, methods, compositions, and kits can be used in conjunction with the sample processing systems and methods, or components thereof (e.g., substrates, detectors, reagent dispensing, continuous scanning, etc.) described herein.

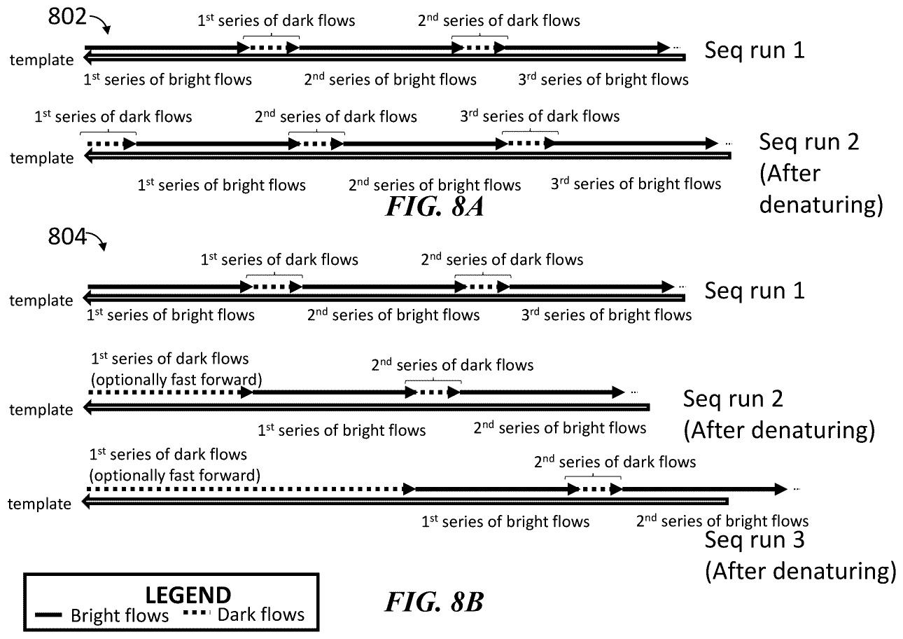

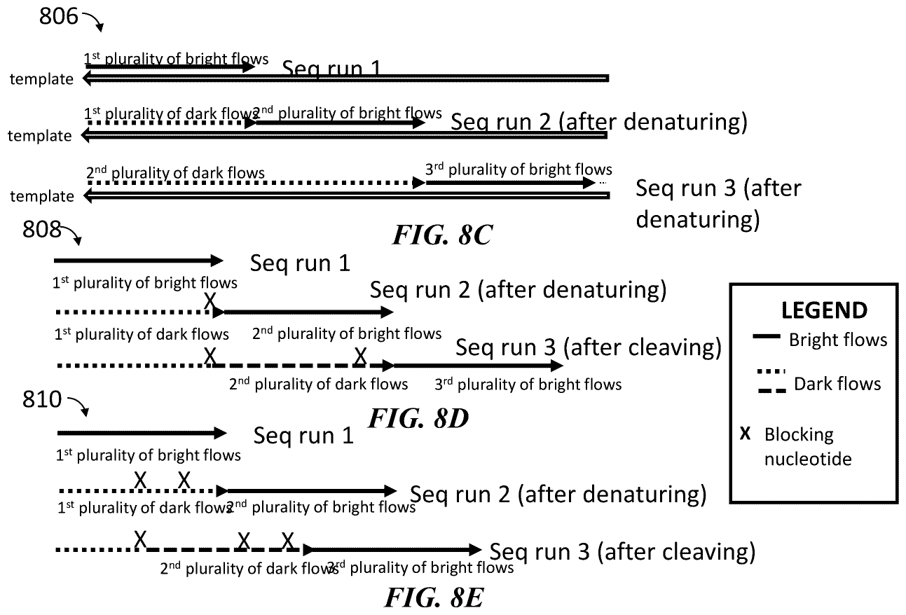

One issue in flow-based sequencing is droop (e.g., the loss in signal intensity over the course of a sequencing run). Droop can be caused by scarring and the stalling of extension of some primers in a colony (e.g., the incorporation of labeled nucleotides into an extending primer and the subsequent removal of a label can result in a chemical scar that inhibits additional nucleotide incorporation into that extending primer). In addition, base call accuracy typically decreases with increasing length of the sequence read (due to e.g., phasing in colony sequencing). Alternatively or in addition, a single sequencing run may fail to determine a complete sequence read (e.g., by ending prematurely) of a full template molecule. This may be due to the stochastic nature of polymerase binding to template molecule (e.g., a polymerase may detach from a template molecule and extending primer and fail to reattach or be replaced by another polymerase) or a cessation of nucleotide incorporation (e.g., as a result of nucleotide scarring from label and/or reversible terminator cleaving). In some cases, it may be advantageous to sequence a template molecule, in whole or in part, multiple times (e.g., to decrease error rates and/or decrease phasing and/or increase overall read length). Different methods may be utilized to reduce scarring and obtain longer sequence reads. For example, in some cases, a sequence read may comprise multiple series of nucleotide flows, where a first set of nucleotide flows comprise at least some labeled nucleotides (e.g., bright flows) and a second set of nucleotide flows comprise unlabeled nucleotides (dark flows). Such sequencing will produce a sequence read including one or more gaps (e.g., sequence information will be obtained for a first region, no information will be gathered for a second intervening region (e.g., the dark flows), sequence information will be obtained for a subsequent third region, etc.). In post-sequencing analysis, these non-contiguous sequencing reads may be associated with a single template molecule, and regions sequenced with unlabeled nucleotides can be inferred by alignment to reference sequences. Alternatively or in addition, a template molecule may be sequenced multiple times (e.g., sequenced via a first sequencing run comprising alternating series of sets of bright and dark flows, having the extended sequencing primer removed, sequenced via a second sequencing run comprising an offset alternating series of sets of bright and dark flows, etc.). This resequencing can increase the confidence in base calls for a sequence run by obtaining repeat information for at least some regions of a template nucleic acid molecule and can enable obtaining a complete sequence for a template nucleic acid molecule without gaps. Repeated sequencing of a same template sequence, either in colony or single molecule sequencing, can alleviate these issues. Exemplary methods for multiple sequencing runs are illustrated by FIGS. 8A-8E.

Re-Sequencing with Alternating Bright and Dark Flows

In some cases, it may be advantageous to sequence a template molecule, in whole or in part, multiple times (e.g., to decrease error rates and/or decrease phasing and/or increase overall read length). For example, typically base call accuracy decreases with increasing length of the sequence read (due to e.g., phasing in colony sequencing). Alternatively or in addition, a single sequencing run may fail to determine a complete sequence read (e.g., by ending prematurely). This may be due to the stochastic nature of polymerase binding to template molecule (e.g., a polymerase may detach from a template molecule and extending primer and fail to reattach or be replaced by another polymerase) or a cessation of nucleotide incorporation (e.g., as a result of nucleotide scarring from label and/or reversible terminator cleaving). Repeated sequencing of a same template sequence, either in colony or single molecule sequencing, can alleviate these issues. Exemplary methods for multiple sequencing runs are illustrated by FIGS. 8A-8E.

In post-sequencing analysis, non-contiguous sequencing reads or multiple sequencing reads may be associated with a single template molecule, and regions of a template molecule sequenced with unlabeled nucleotides (e.g., covered by dark flows) can be inferred by alignment to reference sequences. In some cases, a template molecule may be sequenced multiple times (e.g., sequenced via a first sequencing run, having the extended sequencing primer removed, sequenced via a second sequencing run, etc.), where each or most loci in the template are interrogated via bright flows once (see e.g., FIG. 8C). Extended sequencing primer (e.g., a strand complementary to the template molecule) may be removed via denaturation (e.g., via high temperature, high pH, etc.). For re-sequencing additional sequencing primer may be introduced and hybridized to template molecules after denaturation. In some cases, a template molecule may be sequenced multiple times where at least some loci in the template are interrogated via bright flows more than once (see e.g., FIGS. 8A, 8B). Where a locus in the template molecule is interrogated more than once (e.g., nucleotide incorporation for that locus is detected multiple times), this can increase the confidence in base calls for a sequence run. In some cases, resequencing permits sequencing runs without 100% labeling of nucleotides (e.g., with about 95%, 90%, 85%, 80%, 75%, 70%, 65%, 60%, 55%, 50%, 45%, 40%, 35%, 30%, 25%, 20%, 15%, 10%, 5%, etc. labeled nucleotides per flow). In such cases, after one sequencing run is completed, the extended sequencing primer can be removed in whole or in part (e.g., via thermal denaturing, chemical degradation, enzymatic digestion, etc.). In cases where a sequencing read provides contiguous information for a respective region of a template nucleic acid molecule, in order to determine sequence information for all, substantially all, or a significant portion of a template nucleic acid molecule, multiple sequence reads each providing discrete information may be combined into a single consensus read.

In FIG. 8A, method 802 comprises one or more sequencing runs, each comprising a plurality of alternating series of bright and dark nucleotide flows. Bright nucleotide flows can comprise labeled nucleotides (e.g., flows with at least some and up to 100% labeled nucleotides). Dark nucleotide flows can comprise labeled nucleotides, where incorporation is not detected (e.g., no imaging). Alternatively or in addition, dark nucleotide flows can comprise unlabeled nucleotides. In some cases, a dark flow may comprise a single nucleotide base type. In some cases, a dark flow may comprise a plurality of nucleotide base types (e.g., 2 base types, 3 base types, 4 base types, etc.). In some cases, one or more dark flows may be used (e.g., a first dark flow may comprise a first nucleotide base type comprising unlabeled nucleotides, a second dark flow may comprise three different base types comprising unlabeled nucleotides, and a third dark flow may comprise two different base types where a first base types comprises labeled nucleotides and the second base type comprises unlabeled nucleotides). In some cases, it is sufficient to perform just the first sequencing run in method 802. In such cases, the sequence of regions of the template molecule interrogated with dark flows, can be determined by alignment of the resultant sequence read (e.g., comprising alternating regions with base calls and regions without-corresponding to regions interrogated with labeled and unlabeled nucleotides, respectively). In some cases, multiple sequencing runs are performed, where the series of bright and dark sequencing flows in each run are offset. For instance, the first sequencing run may comprise a first series of 50 bright flows, a first series of 10 dark flows, a third series of 50 bright flows, etc. In some cases, the number of bright flows may be at least 4, 8, 12, 16, 20, 30, 40, 50, 60, 70, 80, 90, or 100 flows. In some cases, the number of intervening dark flows may be at least 4, 8, 12, 16, 20, or 30 flows. In some cases, the number of bright flows may be any number wherein a sequencing polymerase stalls (e.g., where incorporation of labeled nucleotides is inhibited, e.g., by scarring). In some cases, the number of dark flows may be any number that reduces polymerase stalling for subsequent incorporation of labeled nucleotides. In some cases, each series of bright flows may comprise a same number of flows. Alternatively, one or more series of bright flows in a sequencing run may comprise a different number of flows. In some cases, each series of dark flows may comprise a same number of flows. Alternatively, one or more series of dark flows in a sequencing run may comprise a different number of flows. In some cases, dark flows may each comprise a single nucleotide type (e.g., T or C, etc.). In some cases, one or more dark flows may be fast-forward flows comprising more than one nucleotide type (e.g., an A/T/C flow).

In FIG. 8B, method 804 comprises one or more sequencing runs, wherein the first sequencing run is the same as the first sequencing run in method 802. Subsequent sequencing runs may begin with a first series of dark flows (i.e., instead of a first series of bright flows). In some cases, for example in a second sequence run, a first series of dark flows may incorporate the same or approximately the same number of nucleotides as the first series of bright flows in the first sequence run. In some cases, for example in a second sequence run, a first series of dark flows may incorporate an unknown number of nucleotides (e.g., where the first series of dark flows comprise multiple nucleotide types).

In FIG. 8C, method 806 comprises multiple sequencing runs. A first sequencing run may comprise a plurality of bright sequencing flows. Nucleotides added in each bright flow may be labeled (e.g., at least some percentage of the nucleotides in the bright flow may be labeled) and reversibly terminated. Alternatively or in addition, nucleotides added in each bright flow may comprise four, three, two, or one canonical base types. In some cases, nucleotides of each base type may comprise a respective label moiety. In some cases, each respective label moiety may be a different fluorescent label (e.g., fluorescent moieties with different excitation/emission spectra). In some cases, nucleotides of one of the added base types may be unlabeled. In some cases, each respective label moiety may comprise a different number of the same fluorescent label (e.g., where a first base type is labeled with one fluorescent moiety of a first type and a second base type is labeled with three fluorescent moieties of the same type). In some cases, at least a subset of the nucleotides added during bright flows may be labeled and/or unterminated.

A second sequencing run may comprise a plurality of dark sequencing flows, wherein the plurality of dark flows in the second sequencing run comprises the same or similar number of flows as in the plurality of bright flows in the first sequencing run (and/or covers a same or similar region as the bright flows in the first sequencing run). In some cases, nucleotides added in dark flows may be unlabeled and/or unterminated. In some cases, nucleotides added in dark flows may be labeled. In some cases, nucleotides added in dark flows may comprise one, two, three, or four canonical base types. In some cases, in dark flows added nucleotides of a single base type may be terminated (e.g., therefore dark flow nucleotide incorporation will end at that known, terminated base type).

This pattern can be repeated any number of times, with each sequencing run beginning with a successively larger number of dark sequencing flows. For example, the patterns of flows described herein may be repeated any number of times to determine the sequence of a nucleic acid template molecule (e.g., at least 1, 2, 3, 4, 5, 6, 7, 8, 9, 10, 20, 30, 40, 50, 50, 60, 70, 80, 90, 100, 200, 300, 400, 500 or more repetitions). Advantageously, these flow orders may be faster than other multiple sequencing flows (e.g., those described with respect to FIGS. 8A and 8B). Further, using pluralities of dark flows comprising unterminated and/or unlabeled nucleotides can reduce the amount of scarring overall (e.g., each sequencing run may comprise a lower number of scars than if all flows comprised at least some proportion of labeled nucleotides, and a predetermined number of flows for each plurality of bright flows can be selected such that nucleotide incorporation is unlikely to be inhibited by scarring during bright extension steps). The base calls from each sequencing run (e.g., comprising multiple repetitions of bright/dark flow sequencing) can be compiled to form a sequence read.

Sequencing with Blocked Nucleotides

In some cases, a plurality of dark flows may include one or more flows comprising blocking nucleotides. In some cases, blocking nucleotides may comprise cleavable moieties or cross-linking moieties or other linking moieties. A cross-link or other link may be cleaved to release the oligonucleotide molecules, or portion thereof, such as by applying one or more stimuli, including light stimuli, heat stimuli, chemical stimuli, magnetic stimuli, electrical stimuli, and other stimuli, or combination thereof. In some cases, a cross-linking moiety may be photo-cross-linking. A photo-cross-link may be generated by a photo cross-linking reaction. A crosslinking reagent may comprise a photolabile cross-linker, such as a nucleoside analogue 3-Cyanovinylcarbazole nucleosides (^{CNV}K). Photolabile cross-linkers can enable ultra-fast reversible photo-cross-linking of oligonucleotides. When an oligonucleotide strand comprises ^{CNV}K, a cross-link is formed between ^{CNV}K and a pyrimidine base on the complementary strand when illuminated at 365 nm. A complementary nucleotide on the oligonucleotide strand is in 5′ immediately preceding to ^{CNV}K. In some instances, an oligodeoxynucleotide (ODN) comprising 3-cyanovinylcarbazole nucleoside (^{CNV}K) can be subjected to photoirradiation conditions to photo-cross-link a target pyrimidine and the ^{CNV}K. In some instances, irradiation is provided at 366 nm for about 1 second for photo-cross-linking to thymine, and for up to about 25 seconds for photo-cross-linking to cytosine. Complete reversal of the cross-link is achieved by illumination at 312 nm (or at 315 nm) (e.g., irradiation provided at 312 nm for about 3 minutes). Other cross-linkers include, for example, ^{CNV}D, ^{PC}X, or ^{PCX}D. Various other cross-linking reagents may be used to generate a cross-link (e.g., chemical cross-link). In some cases, heat may be applied to denature a double-stranded molecule to facilitate release the unblocked portion of the extended primer from the template molecule (e.g., to facilitate additional sequencing runs). The heat stimulus can be combined with cross-linking reactions. For example, the unblocked portion of the extended primer may be released from the template strand by applying a heat stimulus.

Preferentially, flows comprising denaturation-blocking nucleotides will occur later in a given plurality of dark flows (e.g., resulting in at least one incorporated blocking reagent prior to the succeeding plurality of bright flows). FIGS. 8D and 8E illustrate schema for multiple sequencing runs using blocking nucleotides. These blocking nucleotides (e.g., uracils) may be used as cleavage moieties (e.g., by USER enzyme). This enables bright sequencing flows to restart from the cleaved sites for each additional series of dark flows (e.g., the ‘2^{nd} plurality of dark flows’ in ‘Seq run 2’ in FIG. 8D). Advantageously, this may increase the speed of sequencing (e.g., by decreasing the number of dark flows required in each sequencing run). For example, as illustrated in method 806 in FIG. 8C, the number of flows in the second plurality of dark flows (in sequencing run 3) may be approximately twice the number of flows in the first plurality of dark flows. In contrast, in methods 808 and 810 in FIGS. 8D and 8E respectively, the number of flows in each plurality of dark flows may be approximately the same. In other respects, methods 808 and 810 may be similar to method 806.

In these re-sequencing methods, base calls from multiple sequencing run can be compiled to form a sequence read with an overall decreased error rate. In some cases, sequencing reads corresponding to a same template molecule are identified by barcode sequences. In some cases, sequencing reads are associated by alignment to a reference sequence. In some cases, sequencing reads are identified as corresponding to a same template molecule by location (e.g., signals from multiple sequencing runs may aggregated if the signals are detected at a respective individually addressable location over each sequencing run).

In some cases, a method for sequencing may comprise sequencing a same template strand multiple times to generate robust sequencing data (e.g., a high-quality sequencing read) corresponding to the template strand. In some cases, a method for sequencing may comprise sequencing a same template strand multiple times and sequencing a same reverse complement strand of the template strand multiple times (e.g., both forward and reverse strands) to generate robust sequencing data (e.g., a high-quality paired end read) corresponding to the template strand. A method for re-sequencing a template strand (which may be a forward strand or reverse strand) may comprise annealing a first sequencing primer to the template strand, extending the first sequencing primer through at least a first portion of the template strand via any combination of bright steps and/or dark steps to generate first sequencing data, denaturing the extended strand from the template strand, annealing a second sequencing primer to the template strand, and extending the second sequencing primer through at least a second portion of the template strand via any combination of bright steps and/or dark steps to generate second sequencing data, and processing (e.g., combining, comparing, matching, aligning, resolving, etc.) the first sequencing data and the second sequencing data to generate a sequencing read of the template strand. A template strand may be denatured and re-sequenced any number of times, such as about, at least about, and/or at most about 1, 2, 3, 4, 5, 6, 7, 8, 9, 10 or more times, such as by annealing an nth sequencing primer to the template strand and extending the nth sequencing primer through at least an nth portion of the template strand. The different n sequencing primers may comprise the same or different sequences which may bind to same or different primer binding sites on the template strand, respectively. The different nth portions on the template strand may refer to the same portions or different portions on the template strand. Two portions on the template strand (that are extended through) may be partially overlapping, completely overlapping (for one or both portions), or non-overlapping. The respective extensions through the template strand in the different sequencing runs may use the same or different nucleotide reagents (e.g., non-terminated nucleotides during a first sequencing run, terminated during a second sequencing run; green dye-labeled nucleotides during a first sequencing run, red dye-labeled nucleotides during a second sequencing run; labeled A-, T-, G-bases and unlabeled C-base nucleotides during a first sequencing run, labeled A-, T-, C-bases and unlabeled G-base nucleotides during a second sequencing run; 5% labeled A bases during a first sequencing run; 100% labeled A bases during a second sequencing run; etc.). The respective extensions through the template strand in the different sequencing runs may have the same flow order or flow cycle of nucleotide reagents. The respective extensions through the template strand in the different sequencing runs may have different flow orders or flow cycles of nucleotide reagents (e.g., A->T->G->C single base flow cycle order during a first sequencing run, T->A->G->C single base flow cycle order during a second sequencing run; A/T/G/C 4-base flow cycle order during a first sequencing run; A/T/G->A/T/C 3-base flow cycle order during a second sequencing run, etc.). Denaturing may comprise contacting the double-stranded nucleic acid molecule with denaturing agents, such as sodium hydroxide (NaOH) or ethylene carbonate. An entire substrate may be subjected to resequencing by, after a first sequencing run, contacting the entire surface with a solution comprising a denaturing agent, contacting the entire surface with a solution comprising sequencing primers under conditions sufficient to anneal them to template nucleic acid strands immobilized to the substrate, and subjecting them to extension reactions. In some cases, denaturing may comprise applying heat to the double-stranded nucleic acid molecule.

### Sequencing for Increased Homopolymer Detection Accuracy

### Mixed-Reversibly Terminated Flow Sequencing

In some cases, for a bright sequencing flow, the growing strand (e.g., extending primer) may be contacted with only non-terminated nucleotides-here, if the template has a homopolymer portion, the growing strand may incorporate multiple non-terminated nucleotides in a single step, and thus signals detected from incorporated labeled nucleotides may have to be further resolved to determine the length of the homopolymer. For example, relatively stronger signals may correspond to longer homopolymer length as they are indicative that more labeled nucleotides have been incorporated, and relatively weaker signals may correspond to lower homopolymer length as they are indicative that fewer labeled nucleotides have been incorporated. For example, detected signals may be algorithmically processed to distinguish a 2-mer from a 3-mer or a 4-mer from a 7-mer. However, homopolymer length determination accuracy from these signals may decrease as homopolymer lengths become longer and/or goes above a certain resolution threshold (e.g., 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, etc.), such as due to increasing quenching effects of dye moieties on incorporated labeled nucleotides, optical resolution limitations for signal collection, and/or computing limitations. Alternatively or in addition, nucleotide incorporation may be impeded by the presence of scars in the growing strand (e.g., as a result of cleaving labels from incorporated nucleotides). This can inhibit sequencing, e.g., by increasing phasing, by pausing or stopping incorporation. The present systems, methods, compositions, and kits address at least the abovementioned limitations by improving the accuracy of sequencing reads by reading a homopolymer section of a template in multiple shorter segments and by reducing the impact of scarring. The methods described herein are applicable to either sequencing single molecules or sequencing colonies of amplified template molecules.

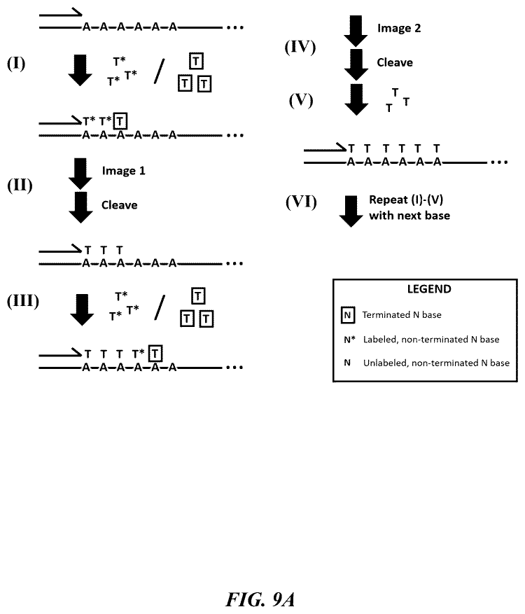

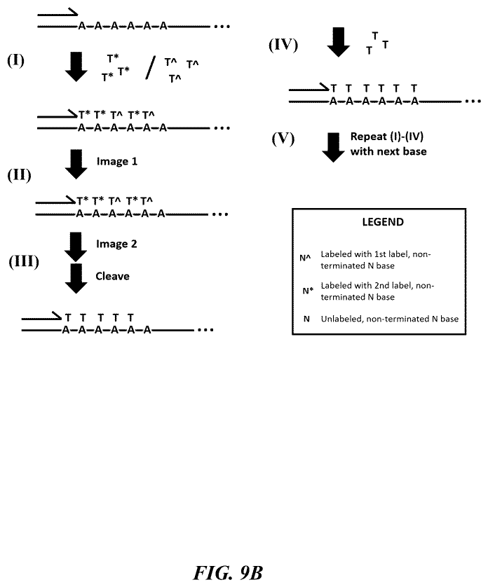

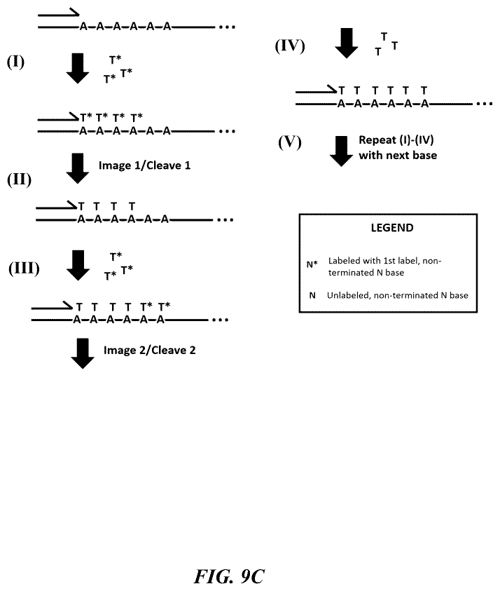

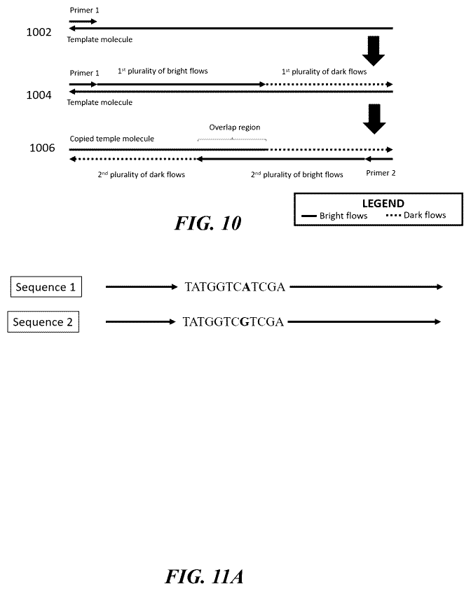

FIG. 9A illustrates an example of a mixed-reversibly terminated sequencing scheme. A template is hybridized to a growing strand which is ready to extend through a 6-mer polyA homopolymer portion in the template. In step (I), the first bright extension step, the growing strand is contacted with a nucleotide mixture comprising both labeled, non-terminated bases and reversibly terminated bases of T. The growing strand incorporates only two labeled, non-terminated T bases before incorporation is blocked by incorporation of a terminated T base, resulting in extending through 3 of 6 available T incorporation positions. In step (II), a first imaging is performed to collect first signals indicative of the first homopolymer segment, and then any labels and blocking moieties removed via cleaving. In step (III), the second bright extension step, step (I) is repeated where the growing strand is contacted with a nucleotide mixture comprising both labeled, non-terminated bases and reversibly terminated bases of T. This time, the growing strand incorporates only one labeled, non-terminated T base before incorporation is blocked by incorporation of a terminated T base, resulting in extending through 2 of 3 of the remaining available T incorporation positions. In step (IV), a second imaging is performed to collect second signals indicative of the second homopolymer segment, and then any labels and blocking moieties removed via cleaving. In step (V), in a dark extension step, the growing strand is contacted with unlabeled, non-terminated T bases to extend through all (in this case 1) of the remaining T incorporation positions. The data collected and/or determined from the two imaging actions (in steps (II) and (IV) respectively) may be processed (e.g., added) to determine a total homopolymer length of the homopolymer portion just sequenced. In this illustration, a determination of at least a 5-mer homopolymer length is made from the data collected. In step (VI), steps (I)-(V) may be repeated with a next, different canonical base type.

It will be appreciated that while this example includes only two bright extension steps ((I)-(II) and (III)-(IV)), any number of bright extension steps may be performed, which can increase the accuracy of the homopolymer length determination.

In some cases, for single molecule sequencing and colony-based sequencing, all non-terminated bases in a bright extension step may be labeled nucleotides. The terminated bases in a bright extension step may be labeled, unlabeled, or a mixture of both. In some cases, in dark extension steps (e.g., step (V)), the growing primer strand is contacted with labeled, non-terminated bases, unlabeled non-terminated bases, or a mixture of labeled and unlabeled non-terminated bases. This may be more efficient in terms of reagent storage space (e.g., obviating the need for separate reagent storage wells for different mixtures of unterminated bases for bright and dark extension steps). Dark extension steps do not include imaging and may include either labeled or unlabeled nucleotides.

In some cases, for colony-based sequencing, the non-terminated bases in a bright extension step may be a mixture of labeled and unlabeled nucleotides. The terminated bases in a bright extension step may be labeled, unlabeled, or a mixture of both. The mixture of labeled and unlabeled nucleotides in the non-terminated bases in the nucleotide reagent may be of any fraction of labeled nucleotides, such as at least or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. The mixture of labeled and unlabeled nucleotides in the terminated bases may be of any fraction of labeled nucleotides, such as at least or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. The mixture of labeled and unlabeled nucleotides in the nucleotide reagent may be of any fraction of labeled nucleotides, such as at least or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. Different fractions of labeled and unlabeled nucleotides, labeled and unlabeled nucleotides in the terminated bases, and/or labeled and unlabeled nucleotides in the non-terminated bases may be different for different base types (e.g., based on expected hmer lengths and/or quenching).

In some cases, for colony-based and single molecule sequencing, the nucleotide reagent can comprise a mixture of terminated and non-terminated nucleotides of any fraction of terminated to non-terminated nucleotides, such as or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. The fraction of terminated nucleotides will influence the average number of bases incorporated in each bright extension step. For example, if the fraction of terminated nucleotides is about 10%, then the average number of incorporated bases in each extending sequencing primer may be about 10 (e.g., 9 incorporated unterminated nucleotides and 1 incorporated terminated nucleotide). Similarly, if the fraction of terminated nucleotides is about 25%, then the average number of incorporated bases may be about 4 (e.g., 3 unterminated nucleotides and 1 terminated nucleotide). At most, one terminated base is expected to be incorporated in each bright extension step.

Any number of consecutive bright extension steps of a same canonical base type may be performed, such as 2, 3, 4, 5, 6, 7, 8 or more consecutive bright extension steps of a same canonical base type. In some cases, the respective number of consecutive bright steps may differ for different nucleotide base types (e.g., 2 consecutive bright steps for A and 3 consecutive bright steps for T). In some cases, a number of consecutive bright steps may be predetermined. In some cases, a number of bright steps may be determined based on relative signal brightness in images of a same nucleotide base type (e.g., Image 1 vs Image 2 in FIG. 9A).

The sequencing method may comprise repeating the subjecting of a growing strand to a template to at least two consecutive bright extension steps followed by a dark extension step of the same canonical base type (e.g., A, G, C, T, U) with different bases for any number of times. For example, at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 600, 700, 800, 900, 1000, 2000, 3000, 4000, 5000, 10,000 or more times.

A sequencing method may comprise subjecting a growing strand hybridized to a template to at least two consecutive bright extension steps followed by a dark extension step of the same canonical base type (e.g., A, G, C, T, U). For sequencing methods described herein, T and U are considered the same canonical base type. A bright extension step may comprise contacting the growing strand with a nucleotide mixture of both (1) labeled, non-terminated bases and (2) reversibly terminated bases of a same canonical base type. The reversibly terminated bases may be labeled or unlabeled, or a mixture of both. In some cases, the last bright extension step may comprise only non-terminated bases and omit the reversibly terminated bases. Beneficially, because of the fraction of reversibly terminated bases in the mixture, when at a long homopolymer stretch in the template, the growing strand is likely to incorporate a reversibly terminated base and block incorporation of the next base before fully extending through a long homopolymer stretch. This allows generating sequencing data by collecting signals (e.g., via imaging) from shorter homopolymer segment intervals, which results in a more accurate homopolymer base call for each segment. Sequencing data generated after each of the bright extension step(s) may be processed (e.g., signals added, images added, homopolymer lengths added, etc.) to determine length information of the homopolymer stretch. For example, a total length of the homopolymer may be determined with high accuracy. In another example, a minimum length of the homopolymer may be determined with high accuracy. Any labels may be removed from the growing strand between different bright extension steps, such as via cleavage, to allow for interval imaging and more efficient incorporation of the next succeeding base. Any blocking moieties may be removed from the growing strand between different extension steps (bright or dark), such as via cleavage, to allow incorporation of the next succeeding base in the next extension step. The bright extension steps may be followed by a dark extension step of the same canonical base type to (1) extend through any remaining portions of a homopolymer stretch that was not covered by the bright extension steps to prepare for interrogation with the next base type and/or (2) catch up any strands (e.g., with a colony) that were unable to incorporate a base(s), such as due to reaction kinetics.

Mixed-Color Flow Sequencing and/or FRET

FIG. 9B illustrates an example of a mixed-color non-terminated sequencing scheme. A template is hybridized to a growing strand which is ready to extend through a 6-mer polyA homopolymer portion in the template. In step (I), the bright extension step, the growing strand is contacted with a nucleotide mixture comprising a first plurality of bases labeled with a first label and a second plurality of bases labeled with a second label, where all of the bases are T. The growing strand incorporates a mixture of Ts with the first and second labels (in this case only 5 Ts are incorporated; in some cases, 6 Ts or 4 Ts may be incorporated). In step (II), a first imaging is performed to collect first signals indicative of the first label. In step (III), a second imaging is performed to collect second signals indicative of the second label, and then any labels are removed via cleaving. In step (IV) (optionally), a dark extension is performed where the growing strand is contacted with unlabeled, non-terminated T bases to extend through (in this case 1) the remaining T incorporation positions. The data collected and/or determined from the two imaging actions (in steps (II) and (III) respectively) may be processed (e.g., added) to determine a total homopolymer length of the homopolymer portion just sequenced.

In some cases, first or second signals may further be indicative of the second or first label, respectively. For example, in some cases, the first label may be a FRET donor, and the second label may be a FRET acceptor (or the reverse). In this illustration, a determination of at least a 5-mer homopolymer length is made from the data collected. In step (V), steps (I)-(IV) may be repeated with a next, different canonical base type. It will be appreciated that while this example includes only two bright extension steps ((I)-(II) and (III)-(IV)), any number of bright extension steps may be performed, which can increase the accuracy of the homopolymer length determination.

Beneficially, the use of at least two label types may improve homopolymer length determination. For instance, there may be less quenching between labels on incorporated nucleotides if there is a mixture of label types. In some cases, 2, 3, 4, 5, 6, 7, 8, 9, 10, or more label types may be used.

In some cases, for single molecule sequencing and colony-based sequencing, all non-terminated bases in a bright extension step may be labeled nucleotides. The non-terminated bases in a bright extension step may be labeled, unlabeled, or a mixture of both. In some cases, in dark extension steps (e.g., step (IV)), the growing primer strand is contacted with labeled, non-terminated bases, unlabeled non-terminated bases, or a mixture of labeled and unlabeled non-terminated bases. This may be more efficient in terms of reagent storage space (e.g., obviating the need for separate reagent storage wells for different mixtures of unterminated bases for bright and dark extension steps). Dark extension steps may not include imaging. As described with respect to FIG. 9A, different proportions of labeled/unlabeled nucleotides may be used. In some cases, different proportions of labeled/unlabeled nucleotides for different canonical base types may be used.

Here a method of sequencing is provided, comprising (a) contacting a growing strand hybridized to a template with a first reagent mixture comprising bases labeled with a first label type and bases labeled with a second label type, wherein the bases are of a first same canonical base type; (b) detecting a first signal indicative of incorporation of at least a subset of the bases labeled with the first label type in the growing strand, or lack thereof, to generate first sequencing data; (c) detecting a second signal indicative of incorporation of at least a subset of the bases labeled with the second label type in the growing strand, or lack thereof, to generate second sequencing data; and (d) processing the first sequencing data and the second sequencing data to determine length information of a homopolymer sequence in the template.

In some cases, the length information of the homopolymer sequence in the template comprises a minimum length of the homopolymer sequence. Alternatively, or in addition, the length information of the homopolymer sequence in the template comprises a total length of the homopolymer sequence.

In some case, the method may further comprise (e) contacting the growing strand with a second reagent mixture comprising unlabeled bases of the first canonical base type. The method may further comprise repeating (a)-(e) with a second canonical base type, a third canonical base type, and/or a fourth canonical base type. These steps may be repeated any number of time suitable for determining the sequence of a nucleic acid template molecule. For example, these steps may be repeated 1, 2, 3, 4, 6, 7, 8, 9, 10, 20, 30, 40, 50, 60, 70, 80, 90, 100, 150, 200, 300, 400, 500, 600, 700, 800, 900, 1000, or more times.

In some cases, the first signal and the second signal may be localized to a single molecule of the template. Alternatively, the first signal and the second signal may be localized to a colony of molecules comprising the template. In some cases, nucleotides are unterminated. In some cases, a mixture of terminated and unterminated nucleotides may be used.

In some cases, the template may be immobilized to a substrate surface. Alternatively or in addition, the template may be coupled to a bead that is immobilized to the substrate surface. Alternatively or in addition, the template may be coupled to a DNA nanoparticle (e.g., a DNA nanoball or DNA origami) that is immobilized to the substrate surface. Alternatively or in addition, the template may be coupled to a dendrimer that is immobilized to the substrate surface. In some cases, the substrate surface comprises at least 1,000,000 individually addressable locations and the template is immobilized to an individually addressable location in the at least 1,000,000 individually addressable locations.

In some cases, multiple labels with similar excitation and emission spectra may be used in combination. In some cases, the use of multiple different labels may reduce quenching between labels on adjacent incorporated nucleotides. That is, in some cases, a method of sequencing is provided, comprising (a) contacting a growing strand hybridized to a template with a reagent mixture comprising bases labeled with a first label type and bases labeled with a second label type, wherein the bases are of a first same canonical base type and wherein the first label type and the second label type may be detected during a same imaging step; (b) detecting a signal indicative of incorporation of at least a subset of the bases labeled with the first label type into the growing strand, at least a subset of the bases labeled with the second label types into the growing strand, or lack thereof, to generate sequencing data; and (c) determining length information of a homopolymer sequence in the template from the sequencing data. The reagent mixture of bases labeled with the first label type and bases labeled with the second label type may be of any fraction of nucleotides with the first label type, such as at least or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%.

### Accumulating Signal Across Multiple Flows

FIG. 9C illustrates an example of a single color non-terminated sequencing scheme. A template is hybridized to a growing strand which is ready to extend through a 6-mer polyA homopolymer portion in the template. In step (I), a first bright extension step is performed where the growing strand is contacted with a nucleotide mixture comprising a first plurality of bases labeled with a first label, where all of the bases are T. The growing strand incorporates a number of Ts less than the respective homopolymer portion in the template (in this case only 4 Ts are incorporated; in some cases, 0, 1, 2, 3, 4, or 5 Ts may be incorporated). In step (II), a first imaging is performed to collect first signals indicative of the first label, and then any labels are removed via cleaving. In step (III), a second bright extension step is performed where the growing strand is contacted with a nucleotide mixture comprising a second plurality of bases labeled with a first label, where all of the bases are T. The growing strand incorporates a number of Ts (in this case 2 Ts are incorporated). In step (IV), a second imaging is performed to collect second signals indicative of the first label, and then any labels are removed via cleaving. In step (V), a dark extension is performed where the growing strand is contacted with unlabeled, non-terminated T bases to extend through (in this case 0) remaining T incorporation positions. The data collected and/or determined from the two imaging actions (in steps (II) and (IV) respectively) may be processed (e.g., added) to determine a total homopolymer length of the homopolymer portion just sequenced. In some cases, the analog signal (e.g., the fluorescence signal detected from labels on incorporated nucleotides) may be added for consecutive imaging steps for a same canonical base type. In some cases, base calls (e.g., based on the respective analog signal) for each incorporation step may be added together for consecutive imaging steps for a same canonical base type. In some cases, no dark extension is performed for one or more canonical base types.

Beneficially, the combination of information from at least two images may improve homopolymer length determination. For instance, there may be less quenching between labels on incorporated nucleotides and/or more linearity of signal strength from labels on incorporated nucleotides if signal may be accumulated across multiple flows and images. In some cases, 2, 3, 4, 5, 6, 7, 8, 9, 10, or more flows and imaging steps may be used for one or more canonical base types. In some cases, in a sequencing run, a same number of flows and imaging steps may be used for each canonical base type (e.g., for each base type, there may be two bright flows each followed by an imaging step). In some cases, in a sequencing run, a different number of flows and imaging steps may be used for one or more canonical base types (e.g., for Ts two bright flows each followed by an imaging step may be used, and for Gs three bright flows each followed by an imaging step may be used).

In some cases, for single molecule sequencing and colony-based sequencing, all non-terminated bases in a bright extension step may be labeled nucleotides. The non-terminated bases in a bright extension step may be labeled, unlabeled, or a mixture of both. In some cases, in dark extension steps (e.g., step (IV)), the growing primer strand is contacted with labeled, non-terminated bases, unlabeled non-terminated bases, or a mixture of labeled and unlabeled non-terminated bases. This may be more efficient in terms of reagent storage space (e.g., obviating the need for separate reagent storage wells for different mixtures of unterminated bases for bright and dark extension steps). Dark extension steps do not include imaging.

Beneficially, the method illustrated in 9C may permit the use of fewer imaging steps for sequencing. This may improve the speed of sequence (e.g., by replacing imaging steps for each extension step with an imaging step for every 2, 3, or 4 extension steps).

Here a method of sequencing is provided, comprising (a) contacting a growing strand hybridized to a template with a first reagent mixture comprising bases labeled with a first label type, wherein the bases are of a first same canonical base type; (b) detecting a first signal indicative of incorporation of at least a subset of the bases labeled with the first label type in the growing strand, or lack thereof, to generate first sequencing data; (c) contacting the growing strand with a second reagent mixture comprising bases labeled with the first label type, wherein the bases are of the first canonical base type; (d) detecting a second signal indicative of incorporation of at least a subset of the bases labeled with the second label type in the growing strand, or lack thereof, to generate second sequencing data; and (e) processing the first sequencing data and the second sequencing data to determine length information of a homopolymer sequence in the template.

In some cases, the length information of the homopolymer sequence in the template comprises a minimum length of the homopolymer sequence. Alternatively, or in addition, the length information of the homopolymer sequence in the template comprises a total length of the homopolymer sequence.

In some case, the method may further comprise (e) contacting the growing strand with a third reagent mixture comprising unlabeled bases of the first canonical base type. The method may further comprise repeating (a)-(e) with a second canonical base type, a third canonical base type, and/or a fourth canonical base type. In some cases, at least a portion of nucleotides in the fourth reaction mixture are labeled. In some cases, in each reaction mixture at least 1% of the nucleotides are labeled. Any percentage of the nucleotides in any reaction mixture may be labeled (with the remaining percentage being unlabeled). In some case, in each reaction mixture 100% of the nucleotides are labeled.

In some cases, all of the label types are excited by the first illumination source. Alternatively, in some cases, each label type is excited by a separate illumination source. In some cases, at least two of the label types may be excited by the first illumination source. In some cases, the first and second label types are excited by a first illumination source, and the third and fourth label types are excited by a second illumination source. Any combination of labels may be excited by a first illumination source (e.g., 1, 2, 3, or 4 labels).

These steps may be repeated any number of time suitable for determining the sequence of a nucleic acid template molecule. For example, these steps may be repeated 1, 2, 3, 4, 6, 7, 8, 9, 10, 20, 30, 40, 50, 60, 70, 80, 90, 100, 150, 200, 300, 400, 500, 600, 700, 800, 900, 1000, or more times.

In some cases, the first signal and the second signal may be localized to a single molecule of the template. Alternatively, the first signal and the second signal may be localized to a colony of molecules comprising the template.

In some cases, nucleotides are unterminated. In some cases, a mixture of terminated and unterminated nucleotides may be used.

In some cases, the template may be immobilized to a substrate surface. Alternatively or in addition, the template may be coupled to a bead that is immobilized to the substrate surface. Alternatively or in addition, the template may be coupled to a DNA nanoparticle (e.g., a DNA nanoball or DNA origami) that is immobilized to the substrate surface. Alternatively or in addition, the template may be coupled to a dendrimer that is immobilized to the substrate surface. In some cases, the substrate surface comprises at least 1,000,000 individually addressable locations and the template is immobilized to an individually addressable location in the at least 1,000,000 individually addressable locations.

In some cases, a combination of any of the systems, methods, and compositions described herein may be used.

Paired End Re-Sequencing with Reversible Terminated Nucleotides

Provided herein are systems, methods, compositions, and kits that enable sequences for improving the accuracy of sequencing reads. In some embodiments, this is achieved by reading a homopolymer section of a template in multiple shorter segments. In some embodiments, this is achieved by reading a homopolymer section using multiple labels simultaneously. Additionally, provided herein are systems, methods, compositions, and kits for improving the speed and accuracy of sequencing with a combination of reversibly terminated and non-incorporable nucleotide analogs. Such systems, methods, compositions, and kits can be applied alternatively or in addition to the sequencing 107 operation described with respect to sequencing workflow 100 of FIG. 1 and, optionally, in the absence of pre-enrichment 102, 103, amplification of templates 105, and/or post-amplification processing 106. Such devices, systems, methods, compositions, and kits can be used in conjunction with the sample processing systems and methods, or components thereof (e.g., substrates, detectors, reagent dispensing, continuous scanning, etc.) described herein.

During sequencing by synthesis, a sequencing primer may be hybridized to a template (e.g., to a primer binding site on the template) and extended in a stepwise manner by, in each step, contacting the hybrid with nucleotide reagents of known canonical base type(s). The extended or extending sequencing primer may also be referred to herein as a growing strand. An extension step may be a bright step (also referred to herein, in some cases, as labeled step or detected step) or a dark step (also referred to herein, in some cases, as an unlabeled step or undetected step). A sequencing method may comprise only bright steps. Alternatively, a sequencing method may comprise a mix of bright step(s) and dark step(s). For a bright step, the growing strand may be contacted with nucleotide reagents that include labeled nucleotides (of known canonical base type(s)) and signals indicative of incorporation of the labeled nucleotides, or lack thereof, may be detected to determine a base or sequence of the template. Alternatively or in addition, for a bright step, the growing strand may be contacted with a mixture of labeled and unlabeled nucleotide reagents. For a dark step, the growing strand may be contacted with solely unlabeled nucleotide reagents. Alternatively or in addition, for a dark step, the growing strand may be contacted with labeled nucleotide reagents and detection omitted.

Flow-based sequencing methods and non-terminated sequencing-by-synthesis methods have been generally described elsewhere herein. In terminated sequencing-by-synthesis methods, a bright step may comprise terminated nucleotides (e.g., reversibly terminated nucleotides). In some cases, a bright step may comprise a mixture of nucleotide base types (e.g., 2, 3, 4, or more base types). A dark step may comprise terminated nucleotides, unterminated nucleotides, or a mixture thereof. A dark step may comprise a single nucleotide base type. Alternatively, a dark step may comprise a mixture of nucleotide base types. In an extension step comprising solely reversibly terminated nucleotides (e.g., and not unterminated nucleotides) a single nucleotide base may be incorporated into a growing strand. In an extension step comprising a mixture of reversibly terminated and unterminated nucleotides, more than one nucleotide base may be incorporated into a growing strand.

FIG. 10 illustrates a schema for paired end sequencing comprising multiple sequencing runs. In step 1002, a first sequencing primer anneals (e.g., is hybridized) to a template molecule at a first primer binding site. For a first number of sequencing flows (e.g., a number of bright flows), or for a first region of the template molecule, labeled and reversibly terminated nucleotides are added and are incorporated into the extending first primer (e.g., nucleotides comprising a labeling moiety and a reversibly terminating moiety). In each of the first number of sequencing flows, each incorporated nucleotide may be detected. After detection, the labeling moiety and/or the terminating moiety is removed (e.g., cleaved) from the incorporated nucleotide.

In some cases, nucleotides added in each bright flow are labeled and reversibly terminated. In some cases, nucleotides added in each bright flow comprise four, three, two, or one canonical base types. In some cases, nucleotides of each base type comprise a respective label moiety. In some cases, each respective label moiety may be a different fluorescent label (e.g., fluorescent moieties with different excitation/emission spectra). In some cases, nucleotides of one of the added base types are unlabeled. In some cases, each respective label moiety may comprise a different number of the same fluorescent label (e.g., where a first base type is labeled with one fluorescent moiety of a first type and a second base type is labeled with three fluorescent moieties of the same type). In some cases, at least a subset of the nucleotides added during bright flows are labeled and/or unterminated.

In step 1004, for a second number of sequencing flows (e.g., a number of dark flows), or for a second region of the template molecule, nucleotides are added and are incorporated into the extending first primer. In the second number of sequencing flows, at most only a subset of incorporated nucleotides is detected. In some cases, nucleotides added in the second number of sequencing flows are labeled and unterminated. In some cases, at least some of the nucleotides in the second number of sequencing flows are unlabeled and unterminated. The dark flows may comprise nucleotides of one, two, three, or four canonical base types. In some cases, nucleotides of one of the added base types are reversibly terminated. In some cases, nucleotides of one or more of the added base types are labeled. In some cases, nucleotides added in dark flows may be unlabeled and/or unterminated.

The extended first sequencing primer comprises a copied template molecule (e.g., a molecule that is complementary to the template molecule). After the second number of sequencing flows (e.g., the dark flows), the copied template molecule and the template molecule are denatured (e.g., exposed to conditions sufficient to denature the copied template molecule from the template molecule).

In step 1006, a second sequencing primer anneals (e.g., is hybridized) to a second sequencing primer binding site in the copied template molecule. The second sequencing primer is extended along the copied template molecule via a first plurality of bright flows followed by a plurality of dark flows. For a first number of sequencing flows (e.g., a number of bright flows), or for a first region of the copied template molecule, labeled and reversibly terminated nucleotides are added and are incorporated into the extending second primer (e.g., nucleotides comprising a labeling moiety and a reversibly terminating moiety). In each of the first number of sequencing flows, each incorporated nucleotide may be detected. After detection, the labeling moiety and/or the terminating moiety is removed (e.g., cleaved) from the incorporated nucleotide. For a second number of sequencing flows (e.g., a number of dark flows), or for a second region of the template molecule, nucleotides are added and are incorporated into the extending second primer. In the second number of sequencing flows, at most only a subset of incorporated nucleotides is detected. That is, detection steps are performed every n flows, where n is an integer greater than 1. N may be 2, 3, 4, 5, 6, 7, 8, 9, 10, etc. In some cases, nucleotides added in the second number of sequencing flows are labeled and unterminated. In some cases, at least some of the nucleotides in the second number of sequencing flows are unlabeled and unterminated. The dark flows may comprise nucleotides of one, two, three, or four canonical base types. In some cases, nucleotides of one of the added base types are reversibly terminated. In some cases, nucleotides of one or more of the added base types are labeled.

The first sequencing primer binding site is located at the 3′ end of the template molecule, and the second sequencing primer binding site is located at the 3′ end of the copied template molecule. In some cases, there will be an overlap in loci covered by bright flows in the template molecule and the copied template molecule (see step 1006). Such loci that have been sequenced twice with bright flows will have decreased base call error rates than loci that were only sequenced once with bright flows.

Re-Sequencing with Different Dark Flow Orders and Detection of Mutations

Multiple sequencing runs (e.g., as illustrated in FIGS. 8A-8E and 9A-9C) can be used to sequence individual template molecules to reduce phasing and incorporation stalling. In some cases, by using a combination of bright steps with reversibly terminated nucleotides and dark steps with unterminated nucleotides, multiple sequencing runs can also provide decreased base error rate information. In particular, the use of different dark step flow orders (e.g., periodic flow orders with one base type provided in each flow), can be used to infer the presence of single nucleotide variants (SNVs) or other variants in regions of a template molecule.

For example, in some cases, a region of a template molecule can be sequenced with a series of sequencing runs comprising: i) extending a hybridized first primer with a first plurality of bright flows, ii) removing the first extended primer and hybridizing a second primer, iii) extending the second primer with a first plurality of dark flows provided in a first flow order, iv) removing the second extended primer and hybridizing a third primer, and v) extending the third primer with a second plurality of dark flows provided in a second flow order different from the first flow order. The base error rate determined for the region of the template molecule may be, on average, lower than if the first region is only interrogated with just bright flows or with bright flows and dark flows in just a first flow order. Alternatively or in addition, a template molecule can be sequenced with a series of sequencing runs comprising: i) extending a hybridized first primer through a first region of the template molecule with a first plurality of bright flows, ii) removing the first extended primer and hybridizing a second primer, iii) extending the second primer with a first plurality of dark flows provided in a first flow order, iv) removing the second extended primer and hybridizing a third primer, v) extending the third primer through a second region of the template molecule with a second plurality of bright flows, vi) removing the third extended primer and hybridizing a fourth primer, and vii) extending the fourth primer with a second plurality of dark flows provided in a second flow order different from the first flow order. Bright extension steps comprise terminated and labeled nucleotides, where multiple nucleotide base types may be added concurrently. For the detection of SNVs, dark extension steps may comprise unterminated nucleotides added sequentially.

The second primer may be extended through the first region of the template molecule, through most of the first region, or through more than the first region of the template molecule. The fourth primer may be extended through both the first and second regions of the template molecule, through the first region and most of the second, or through the first region and more than the second region. The base error rate determined for the first and second regions of the template molecule may be, on average, lower than if the first and second regions were interrogated with just bright flows or with bright flows and dark flows in just a first flow order.

For interrogating a given region of a template molecule, and for a plurality of bright flows comprising n flows (e.g., where n nucleotides are expected to be incorporated), a corresponding plurality of dark flows comprising x flows may be provided, where approximately m nucleotides are expected to be incorporated. For instance, a four base periodic flow order of unterminated nucleotides may have a base efficiency around 0.6 (i.e., for every flow on average 0.6 nucleotides are incorporated into an extending primer sequence, see Table 1). With reversibly terminated nucleotides, in each flow, a single nucleotide is incorporated into an extending primer sequence (barring errors by polymerases, issues with template molecules, incomplete or early cleavage of terminators, etc.). Thus, more dark flows than bright flows may be needed to cover a same region of a template molecule. Similarly, m may be less than, equal to, or greater than n. Where m is less than or greater than n, it may be inferred that a SNP is present.

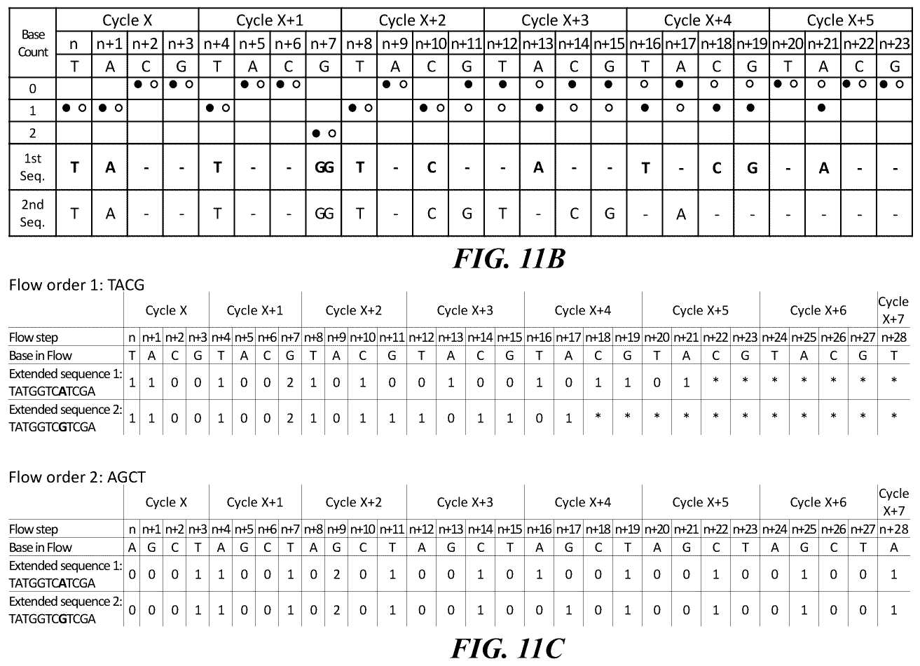

By way of example, FIG. 11A shows two extended primer sequences (e.g., reverse complements of respective template molecules) that differ at one locus. Sequence 1 contains TATGGTCATCGA (SEQ ID NO: 1) and Sequence 2 contains TATGGTCGTCGA (SEQ ID NO: 2). FIG. 11B illustrates the nucleotide incorporation of Sequence 1 and Sequence 2 respectively, using a same dark flow order (e.g., unterminated nucleotides T-A-C-G), starting at cycle n in cycle X. In cycle X+2 of the flow order, the two sequences begin to diverge (e.g., after incorporation of C). After flow n+10, in Sequence 2, a G is incorporated in flow n+11, while in Sequence 1 no additional nucleotides are incorporated until flow n+13. Sequence 2 is completed in cycle X+4 while Sequence 1 is not completed until cycle X+5. This illustrates that different sequences can, when extended using a same non-terminated nucleotide flow order, require a different number of flows (e.g., incorporation steps). Thus, Sequences 1 and 2 will be offset in all future flow steps (e.g., dark and then bright flow steps). The difference between these sequences will not be evident until incorporation signal is obtained (e.g., from imaging bright incorporation steps).

The top panel in FIG. 11C is an extension of the data in FIG. 11B. The lower panel in FIG. 11C illustrates nucleotide incorporation for Sequences 1 and 2 using a different flow order (e.g., A-G-C-T). As discussed above, using the first flow order (T-A-C-G) reveals a difference between these two sequences. However, despite the A->G substitution, a same number of flow cycles (e.g., 29 flow steps across 8 flow cycles) are used to extend both Sequences 1 and 2. This illustrates that the use of different flow orders can be helpful in revealing SNPs. Preferentially, multiple different flow orders (e.g., at least 2) may be used to interrogate a template molecule.

In some cases, resequencing with different flow orders, especially resequencing with bright labeled and terminated flows in combination with dark flow orders with different series of unterminated nucleotide base types, may be used in combination with other methods described herein (e.g., as described with respect to FIGS. 8A-8E and 9A-9C). By way of example, in FIG. 8C, in sequence run 1, the first plurality of bright flows may comprise labeled and terminated nucleotides, where A, T, C, and G nucleotides are added concurrently in each flow. The first plurality of bright flows may comprise 60 flows (e.g., leading to the incorporation of 60 nucleotides into the growing strand (e.g., the extending primer). In sequence run 2, the first plurality of dark flows may comprise unlabeled and unterminated nucleotides, where the nucleotide base types are added sequentially according to a first flow order T-A-C-G. Assuming the T-A-C-G flow order has a 60% efficiency (e.g., 6 nucleotides incorporated on average per 10 flows), then the first plurality of dark flows may comprise 100 flows. Thus, the first plurality of dark flows and the first plurality of bright flows may incorporate approximately the name number of nucleotides (e.g., the second plurality of bright flows may begin approximately at the same locus in the template molecule where the first plurality of bright flows ended). The second plurality of bright flows in sequence run 2 may comprise labeled terminated nucleotides, where all nucleotide base types are added concurrently in each flow. In sequence run 3, the second plurality of dark flows may comprise unlabeled and unterminated nucleotides, where the nucleotide base types are added sequentially according to a second flow order A-G-C-T.

Assuming that the second flow order has a 60% efficiency, and if the second plurality of bright flows comprises 60 flows, then the second plurality of dark flows may comprise 100 flows (e.g., the third plurality of bright flows may begin approximately at the same locus in the template molecule where the second plurality of bright flows ended).

### Paired End Flow Sequencing Methods

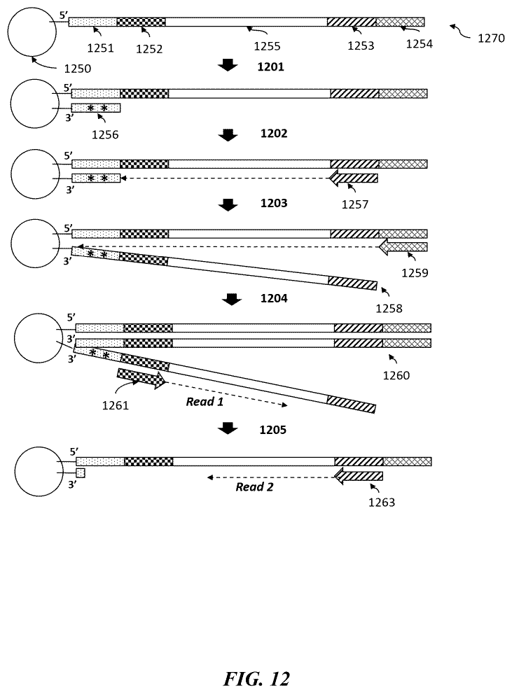

Provided herein are systems and methods for obtaining paired end reads. FIGS. 12-QZ8 illustrate various methods for obtaining paired end reads from a template molecule while sequencing on a support. Paired end flow sequencing may be performed in combination with any other sequencing method described herein.

FIG. 12 shows an oligonucleotide molecule 1270 whose 5′ end is immobilized to a support 1250, such as a bead. The oligonucleotide molecule may be covalently or non-covalently bound to the support. The oligonucleotide molecule may comprise, in order of 5′ to 3′, a first adaptor 1251, a second adaptor 1252, a template insert sequence 1255, a third adaptor 1253, and a fourth adaptor 1254. In 1201, an additional oligonucleotide molecule 1256 may be immobilized to the support at 3′ end. The additional oligonucleotide molecule may be covalently or non-covalently bound to the support via any coupling mechanism described elsewhere herein. For example, in some cases, the additional oligonucleotide molecule may be attached via click chemistry, such as via a first reactive moiety on the support and a second reactive moiety at a 3′ end of the additional oligonucleotide molecule. Example click chemistry pairs are described elsewhere herein (e.g., DBCO-azide pair). The additional oligonucleotide molecule may be at least partially complementary to and hybridized to the first adaptor of the oligonucleotide molecule. The additional oligonucleotide molecule may comprise one or more cleavable moieties (denoted “*” in the figure). The cleavable moiety may be any cleavable or excisable moiety described elsewhere herein (e.g., uracil, ribonucleotide, etc.).

Then, in 1202, a first extension primer 1257 which is at least partially complementary to the third adaptor 1253 may be hybridized to the third adaptor of the oligonucleotide molecule, extended towards the additional oligonucleotide molecule 1256, and joined to the additional oligonucleotide molecule via ligation to generate first extended molecule 1258 that is hybridized to the oligonucleotide molecule 1270. In 1203, a second extension primer 1259 which is at least partially complementary to the fourth adaptor 1254 may be hybridized to the fourth adaptor of the oligonucleotide molecule and extended towards 5′ end of the oligonucleotide molecule with a strand displacing polymerase to generate a second extended molecule 1260, in the process displacing the first extended molecule 1258 and rendering the first extended molecule at least partially single-stranded near its 5′ end.

In 1204, a first sequencing primer 1261 may bind to the displaced first extended molecule 1258 and be extended in a sequencing reaction to generate a first sequencing read comprising a first sequence corresponding to a sequence of the template insert sequence 1255 in 5′ to 3′ direction. The first sequencing primer may be at least partially complementary to and bind to a region of the first extended molecule 1258 that corresponds to the second adaptor 1252 of the oligonucleotide molecule (the first sequencing primer binding region may comprise a reverse complement of the second adaptor). In 1205, the one or more cleavable moieties in the additional oligonucleotide molecule 1256 in the first extended molecule 1258 may be cleaved, thus releasing the first extended molecule, or at least a portion (closer to 5′ end) thereof, from the support 1250. In some cases, the entire additional oligonucleotide molecule may be digested. In other cases, a portion (e.g., closer to the 3′ end) may remain bound to the support. Double-stranded molecules on the support may be subjected to stripping, such as via NaOH treatment, to remove hybridized strands. In the illustration, after stripping, the oligonucleotide molecule 1270 remains bound to the support 1250. Optionally, a portion of the additional oligonucleotide molecule 1256 (closer to the 3′ end) may also remain bound to the support. A second sequencing primer 1263 may bind to the oligonucleotide molecule 1270 and be extended in a sequencing reaction to generate a second sequencing read comprising a second sequence corresponding to a sequence of the template insert sequence 1255 in 3′ to 5′ direction. The second sequencing primer may be at least partially complementary to and bind to the third adaptor 1253. The first sequencing read and the second sequencing read may be paired and processed to generate a paired read of the template insert sequence 1255.

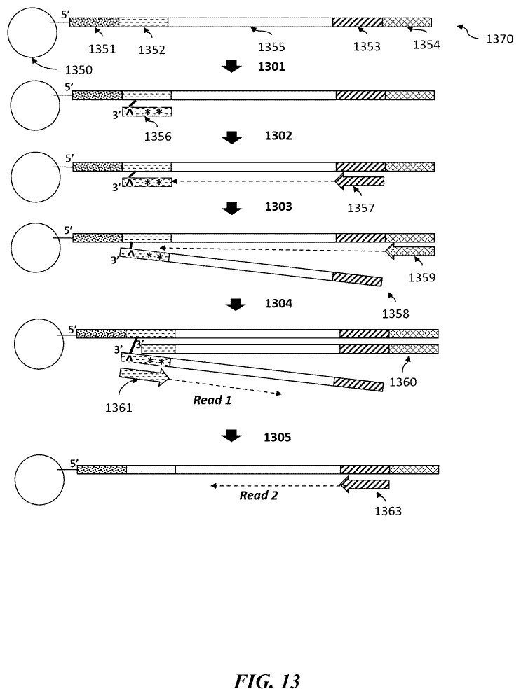

FIG. 13 shows an oligonucleotide molecule 1370 whose 5′ end is immobilized to a support 1350, such as a bead. The oligonucleotide molecule may be covalently or non-covalently bound to the support. The oligonucleotide molecule may comprise, in order of 5′ to 3′, a first adaptor 1351, a second adaptor 1352, a template insert sequence 1355, a third adaptor 1353, and a fourth adaptor 1354. In 1301, an additional oligonucleotide molecule 1356 may be hybridized to at least a portion of the second adaptor 1352 region of the oligonucleotide molecule. The additional oligonucleotide molecule may be at least partially complementary to the second adaptor. The additional oligonucleotide molecule and the oligonucleotide may be coupled in addition to the hybridization, such as via crosslinking, click chemistry, or any other coupling mechanism described elsewhere herein. The coupling may be covalent or non-covalent. In the illustration, as an example, the additional oligonucleotide molecule comprises one or more coupling moieties (denoted “\hat{ }” in the figure). The one or more coupling moieties may comprise a cross-linking reagent (e.g., ^{CNV}K) that crosslinks to a base in the second adaptor. The one or more coupling moieties may comprise any other moiety (e.g., sulfur for S—S bonding, click chemistry reagents, etc.). The coupling may be activated, such as via light treatment or application of one or more stimuli (e.g., heat, light, chemical reagent, etc.). The coupling may be reversible or irreversible. In some cases, the additional oligonucleotide molecule may comprise one or more cleavable moieties (denoted “*” in the figure), e.g., that is disposed 5′ to the cross-linking reagent. The cleavable moiety may be any cleavable or excisable moiety described elsewhere herein (e.g., uracil, ribonucleotide, etc.).

Then, in 1302, a first extension primer 1357 which is at least partially complementary to the third adaptor 1353 may be hybridized to the third adaptor of the oligonucleotide molecule, extended towards the additional oligonucleotide molecule 1356, and joined to the additional oligonucleotide molecule via ligation to generate first extended molecule 1358 that is hybridized to the oligonucleotide molecule 1370. In 1303, a second extension primer 1359 which is at least partially complementary to the fourth adaptor 1354 may be hybridized to the fourth adaptor of the oligonucleotide molecule and extended towards 5′ end of the oligonucleotide molecule with a strand displacing polymerase to generate a second extended molecule 1360, in the process displacing the first extended molecule 1358 and rendering the first extended molecule 1358 at least partially single-stranded near its 5′ end. The first extended molecule 1358 may still remain immobilized to the support 1350 via its coupling (e.g., cross-linking) to the oligonucleotide molecule 1370.

In 1304, a first sequencing primer 1361 may bind to the displaced first extended molecule 1358 and be extended in a sequencing reaction to generate a first sequencing read comprising a first sequence corresponding to a sequence of the template insert sequence 1355 in 5′ to 3′ direction. The first sequencing primer may be at least partially complementary to and bind to a region of the first extended molecule 1358 that corresponds to the second adaptor 1352 of the oligonucleotide molecule (the first sequencing primer binding region may comprise a reverse complement of the second adaptor). In 1305, the coupling (e.g., cross-linking) between the oligonucleotide molecule 1370 and the additional oligonucleotide molecule 1356 may be reversed and/or one or more cleavable moieties in the additional oligonucleotide molecule 1356 in the first extended molecule 1358 may be cleaved, thus releasing the first extended molecule 1358, or at least a portion (closer to 5′ end) thereof, from the support 1350. In some cases, the entire additional oligonucleotide molecule may be released. In other cases, a portion (e.g., closer to the 3′ end) may remain coupled to the oligonucleotide molecule 1370. Double-stranded molecules on the support may be subjected to stripping, such as via NaOH treatment, to remove hybridized strands. In the illustration, after stripping, the oligonucleotide molecule 1370 remains bound to the support 1350. A second sequencing primer 1363 may bind to the oligonucleotide molecule 1270 and be extended in a sequencing reaction to generate a second sequencing read comprising a second sequence corresponding to a sequence of the template insert sequence 1355 in 3′ to 5′ direction. The second sequencing primer may be at least partially complementary to and bind to the third adaptor 1353. The first sequencing read and the second sequencing read may be paired and processed to generate a paired read of the template insert sequence 1355.

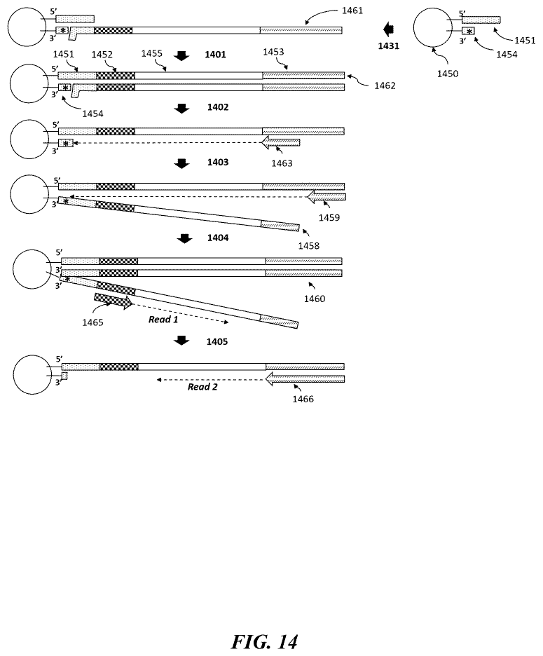

FIG. 14 provides a starting support 1450 that comprises a first surface oligonucleotide molecule 1451 and a second surface oligonucleotide molecule 1454 each coupled to the support at the 5′ end and 3′ end, respectively. The coupling may be covalent or non-covalent via any coupling mechanism described elsewhere herein (e.g., click chemistry). The first and second surface oligonucleotide molecules may have different lengths. The second surface oligonucleotide may be shorter than the first surface oligonucleotide. The second surface oligonucleotide may be hybridized to the first surface oligonucleotide. In some cases, the second surface oligonucleotide may comprise one or more cleavable moieties (denoted “*” in the figure). The cleavable moiety may be any cleavable or excisable moiety described elsewhere herein (e.g., uracil, ribonucleotide, etc.).

In 1431, a library molecule 1461 comprising a binding sequence near 3′ end that is at least partially complementary to the first surface oligonucleotide molecule 1451 may contact the starting support 1450, such that at least a portion of the binding sequence hybridizes to the first surface oligonucleotide molecule. In some cases, a 3′ end of the library molecule may not bind to the first surface oligonucleotide molecule, such as due to the second surface oligonucleotide molecule already hybridized to the first surface oligonucleotide molecule and remain as a single-stranded overhang. In 1401, the first surface oligonucleotide molecule may be extended using the library molecule 1461 as a template to generate oligonucleotide molecule 1462. The oligonucleotide molecule may comprise from a 5′ to 3′ direction, the first surface oligonucleotide molecule 1451 (or a first adaptor), a second adaptor 1452, a template insert sequence 1455, and a third adaptor 1453.

Then, in 1402, double-stranded molecules on the support may be subjected to stripping, such as via NaOH treatment, to remove hybridized strands, thus removing library molecule 1461 from the support 1450. A first extension primer 1463 which is at least partially complementary to a 5′ region of the third adaptor 1453 may be hybridized to the third adaptor of the oligonucleotide molecule, extended towards the second surface oligonucleotide molecule 1454, and joined via ligation to generate first extended molecule 1458 that is hybridized to the oligonucleotide molecule 1462. In 1403, a second extension primer 1459 which is at least partially complementary to a 3′ region of the third adaptor 1453 may be hybridized to the third adaptor of the oligonucleotide molecule and extended towards 5′ end of the oligonucleotide molecule with a strand displacing polymerase to generate a second extended molecule 1460, in the process displacing the first extended molecule 1458 and rendering the first extended molecule 1458 at least partially single-stranded near its 5′ end.

In 1404, a first sequencing primer 1465 may bind to the displaced first extended molecule 1458 and be extended in a sequencing reaction to generate a first sequencing read comprising a first sequence corresponding to a sequence of the template insert sequence 1455 in 5′ to 3′ direction. The first sequencing primer may be at least partially complementary to and bind to a region of the first extended molecule 1458 that corresponds to the second adaptor 1452 of the oligonucleotide molecule (the first sequencing primer binding region may comprise a reverse complement of the second adaptor). In 1405, the one or more cleavable moieties in the second surface oligonucleotide molecule 1454 in the first extended molecule 1458 may be cleaved, thus releasing the first extended molecule 1458, or at least a portion (closer to 5′ end) thereof, from the support 1450. In some cases, the entire second surface oligonucleotide molecule may be digested. In other cases, a portion (e.g., closer to the 3′ end) may remain bound to the support. Double-stranded molecules on the support may be subjected to stripping, such as via NaOH treatment, to remove hybridized strands. In the illustration, after stripping, the oligonucleotide molecule 1462 remains bound to the support 1450. Optionally, a portion of the second surface oligonucleotide molecule 1454 (closer to the 3′ end) may also remain bound to the support. A second sequencing primer 1466 may bind to the oligonucleotide molecule 1462 and be extended in a sequencing reaction to generate a second sequencing read comprising a second sequence corresponding to a sequence of the template insert sequence 1455 in 3′ to 5′ direction. The second sequencing primer may be at least partially complementary to and bind to the third adaptor 1453. The first sequencing read and the second sequencing read may be paired and processed to generate a paired read of the template insert sequence 1455.

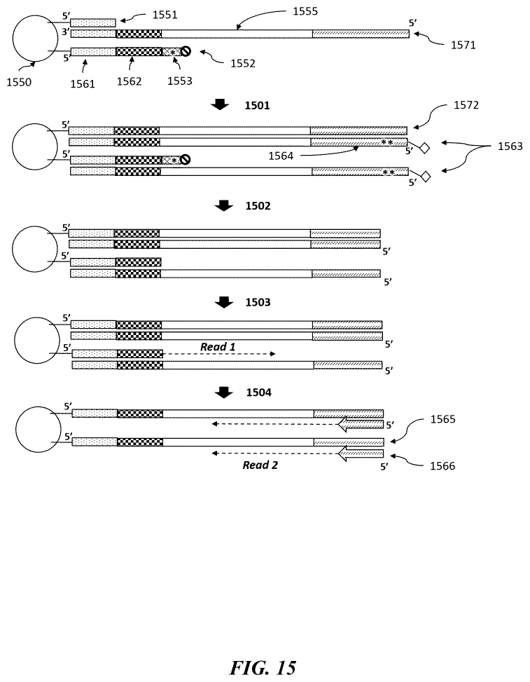

FIG. 15 shows a first surface oligonucleotide 1551 and a second surface oligonucleotide 1552 each immobilized to a support 1550, such as a bead, at its 5′ end. The first surface oligonucleotide may be an unblocked primer, capable of extension. The second surface oligonucleotide may be a blocked primer, blocked from extension. The support may comprise any % of first surface oligonucleotides (e.g., unblocked primers) and % of second surface oligonucleotides (e.g., blocked primers). For example, the support may comprise about, at least about, and/or at most about 5%, 10%, 15%, 20%, 25%, 30%, 35%, 40%, 45%, 50%, 55%, 60%, 65%, 70%, 75%, 80%, 85%, 90%, 95% or more first or second surface oligonucleotides out of all total surface oligonucleotides. The first and/or second surface oligonucleotides may be covalently or non-covalently bound to the support. The second surface oligonucleotide 1552 may comprise, in order of 5′ to 3′, a first adaptor 1561, a second adaptor 1562, and a blocking mechanism 1553, the blocking mechanism comprising a blocker at or adjacent to the 3′ end. For example, the blocker may be a dideoxynucleotide, another terminator, polymer, spacer, abasic site, or other entity. The blocking mechanism may comprise one or more bases, with the blocker disposed at or adjacent to the 3′ end. The second surface oligonucleotide may comprise one or more cleavable moieties (denoted “*”) disposed 5′ to the blocker, such as in the blocking mechanism 1553 or another portion of the second surface oligonucleotide. The cleavable moiety may be any cleavable or excisable moiety described elsewhere herein (e.g., uracil, ribonucleotide, etc.). In some cases, the first adaptor 1561 may have the same and/or at least partially overlapping sequence as the first surface oligonucleotide 1551. A library molecule 1571 which is at least partially complementary to the first surface oligonucleotide 1551 may be hybridized to the first surface oligonucleotide and/or the second surface oligonucleotide 1552 (the figure illustrating hybridization with only the first surface oligonucleotide). The library molecule may be a single stranded DNA template comprising insert sequence 1555.

In 1501, the library molecule 1571 may be amplified to generate copies and/or reverse complement copies that are bound, covalently or non-covalently (e.g., hybridized), to the support 1550. For example, during amplification, the first surface oligonucleotide 1551 may be extended using the library molecule 1571 as a template to generate first extended surface molecule 1572. Double-stranded molecules on the support may be subjected to stripping, such as via NaOH treatment, to strip away the library molecule 1571 from the support. In place, extension primer 1564 may be annealed to a 3′ end of the first extended surface molecule and extended using the first extended surface molecule as a template to generate second extended molecule 1563. The second extended molecule 1563 may be denatured from the first extended surface molecule and hybridized to the second surface oligonucleotide 1552. This process may be repeated any number of times to generate multiple first extended surface molecules (e.g., 1562, extended from other first surface oligonucleotides on the support) and multiple second extended molecules (e.g., 1563) hybridized to the first extended surface molecules and/or second surface oligonucleotides. Optionally, extension primer 1564 may comprise a capture entity that may be used to enrich a mixture of supports post-amplification to isolate for positive supports (comprising amplified copies of library molecules). Any capture and capturing mechanism described elsewhere herein may be used, such as biotin and streptavidin binding. Optionally, extension primer 1564 may comprise one or more cleavable moieties disposed 3′ of a capture entity (e.g., biotin) such that the capture entity can be cleaved from the support by cleaving or excising the one or more cleavable moieties, such as after enrichment.

Then, in 1502, the one or more cleavable moieties (“*”) in the second surface oligonucleotide 1552 may be cleaved or otherwise removed to unblock the second surface oligonucleotide, providing an unblocked second primer on the support 1550 that is capable of extending. In 1503, the unblocked second primer may be used as a first sequencing primer and be extended in a sequencing reaction, using the second extended molecule hybridized to the second surface oligonucleotide 1552 as a template, to generate a first sequencing read comprising a first sequence corresponding to a sequence of the template insert sequence 1555 in the 3′ to 5′ direction. The first sequencing primer may be extended to generate a third extended surface molecule 1565. The third extended surface molecule may comprise the same sequence as first extended surface molecule 1563. In 1504, double-stranded molecules on the support may be subjected to stripping, such as via NaOH treatment, to remove hybridized strands (e.g., second extended oligonucleotide molecules). In the illustration, after stripping, the extended surface molecules (e.g., first and third extended surface molecules 1563, 1565) remain bound to the support 1550. A second sequencing primer 1566 may bind to the extended surface molecules at the 3′ end and be extended in a sequencing reaction to generate a second sequencing read comprising a second sequence corresponding to a sequence of the template insert sequence 1555 in 5′ to 3′ direction. The first sequencing read and the second sequencing read may be paired and processed to generate a paired read of the template insert 1555.

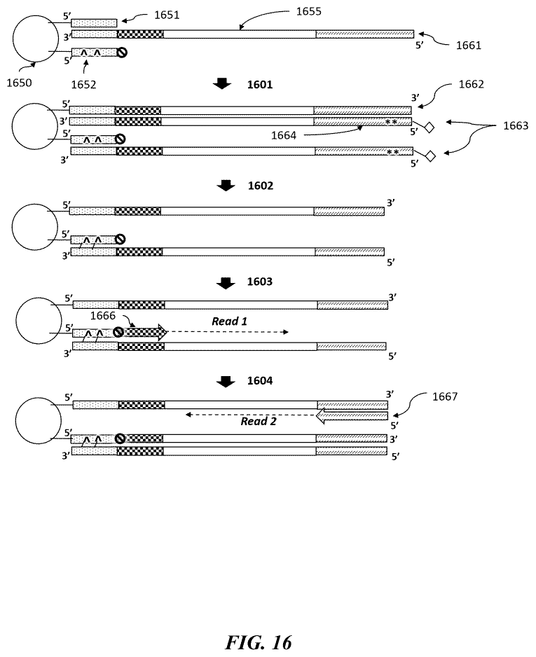

FIG. 16 shows a first surface oligonucleotide 1651 and a second surface oligonucleotide 1652 each immobilized to a support 1650, such as a bead, at its 5′ end. The first surface oligonucleotide may be an unblocked primer, capable of extension. The second surface oligonucleotide may be a blocked primer, blocked from extension. The support may comprise any % of first surface oligonucleotides (e.g., unblocked primers) and % of second surface oligonucleotides (e.g., blocked primers). The first and/or second surface oligonucleotides may be covalently or non-covalently bound to the support. The second surface oligonucleotide may comprise a blocking mechanism that comprises a blocker at or adjacent to the 3′ end. For example, the blocker may be a dideoxynucleotide, another terminator, polymer, spacer, abasic site, or other entity. The blocking mechanism may comprise one or more bases, with the blocker disposed at or adjacent to the 3′ end. The second surface oligonucleotide may comprise one or more coupling moieties (denoted “\hat{ }”) that can permit coupling of the second surface oligonucleotide to another oligonucleotide alternatively or in addition to hybridization. For example, the one or more coupling moieties may comprise a cross-linking reagent (e.g., ^{CNV}K) that crosslinks to a base in the other oligonucleotide. Any other coupling mechanism described elsewhere herein, covalent or noncovalent, may be used. The one or more coupling moieties may comprise any other moiety (e.g., sulfur for S—S bonding, click chemistry reagents, etc.). The coupling may be activated, such as via light treatment or application of one or more stimuli (e.g., heat, light, chemical reagent, etc.). The coupling may be reversible or irreversible. In some cases, the second surface oligonucleotide 1652 may have the same and/or at least partially overlapping sequence as the first surface oligonucleotide 1651. A library molecule 1661 which is at least partially complementary to the first surface oligonucleotide 1651 may be hybridized to the first surface oligonucleotide and/or the second surface oligonucleotide 1652 (the figure illustrating hybridization with only the first surface oligonucleotide). The library molecule may be a single stranded DNA template comprising insert sequence 1655.

In 1601, the library molecule 1661 may be amplified to generate copies and/or reverse complement copies that are bound, covalently or non-covalently (e.g., hybridized), to the support 1650. For example, during amplification, the first surface oligonucleotide 1651 may be extended using the library molecule 1661 as a template to generate first extended surface molecule 1662. Double-stranded molecules on the support may be subjected to stripping, such as via NaOH treatment, to strip away the library molecule 1661 from the support. In place, extension primer 1664 may be annealed to a 3′ end of the first extended surface molecule and extended using the first extended surface molecule as a template to generate second extended molecule 1663. The second extended molecule 1663 may be denatured from the first extended surface molecule and hybridized to the second surface oligonucleotide 1652. This process may be repeated any number of times to generate multiple first extended surface molecules (e.g., 1662, extended from other first surface oligonucleotides on the support) and multiple second extended molecules (e.g., 1663) hybridized to the first extended surface molecules and/or second surface oligonucleotides. Optionally, extension primer 1664 may comprise a capture entity that may be used to enrich a mixture of supports post-amplification to isolate for positive supports (comprising amplified copies of library molecules). Any capture and capturing mechanism described elsewhere herein may be used, such as biotin and streptavidin binding. Optionally, extension primer 1664 may comprise one or more cleavable moieties disposed 3′ of a capture entity (e.g., biotin) such that the capture entity can be cleaved from the support by cleaving or excising the one or more cleavable moieties, such as after enrichment.

Then, in 1602, the one or coupling moieties (“\hat{ }”) in the second surface oligonucleotide 1652 may be activated to couple the second surface oligonucleotide to the second extended molecule 1663, in addition to hybridization. For example, cross-linking may be activated via light treatment. After coupling, double-stranded molecules on the support may be subjected to stripping, such as via NaOH treatment, to strip away any hybridized second extended molecules (e.g., 1663) that is not otherwise coupled (e.g., cross-linked) to second surface oligonucleotides (e.g., 1652). After stripping, the support 1650 may comprise first extended surface molecules (e.g., 1662) and only those second extended molecules (e.g., 1663) coupled to second surface oligonucleotides (e.g., 1652). In 1603, a first sequencing primer 1666 may hybridize to the second extended molecule 1663 near a 3′ end of the second extended molecule and be extended in a sequencing reaction to generate a first sequencing read comprising a first sequence corresponding to a sequence of the template insert sequence 1555 in 3′ to 5′ direction. The first sequencing primer may hybridize to a region in the second extended molecule that is 5′ to a coupling site between the second extended molecule and the second surface oligonucleotide 1652. In 1604, a second sequencing primer 1667 may bind to the first extended surface molecules 1662 at or adjacent to the 3′ end and be extended in a sequencing reaction to generate a second sequencing read comprising a second sequence corresponding to a sequence of the template insert 1655 in 5′ to 3′ direction. The first and second sequencing reads may be paired and processed to generate a paired read of the template insert 1655.

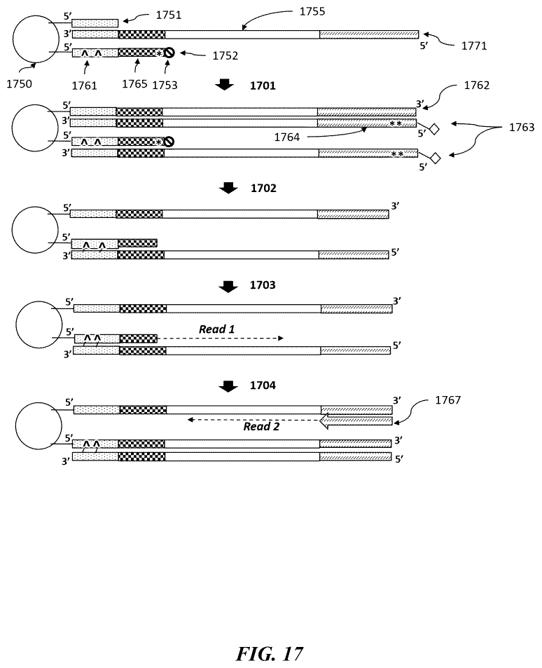

FIG. 17 shows a first surface oligonucleotide 1751 and a second surface oligonucleotide 1752 each immobilized to a support 1750, such as a bead, at its 5′ end. The first surface oligonucleotide may be an unblocked primer, capable of extension. The second surface oligonucleotide may be a blocked primer, blocked from extension. The support may comprise any % of first surface oligonucleotides (e.g., unblocked primers) and % of second surface oligonucleotides (e.g., blocked primers). The first and/or second surface oligonucleotides may be covalently or non-covalently bound to the support. The second surface oligonucleotide may comprise, in order of 5′ to 3′, a first adaptor 1761, a second adaptor 1765, and a blocking mechanism 1753, the blocking mechanism comprising a blocker at or adjacent to the 3′ end. For example, the blocker may be a dideoxynucleotide, another terminator, polymer, spacer, abasic site, or other entity. The blocking mechanism may comprise one or more bases, with the blocker disposed at or adjacent to the 3′ end. The second surface oligonucleotide may comprise one or more cleavable moieties (denoted “*”) disposed 5′ to the blocker, such as in the second adaptor 1765 or another portion of the second surface oligonucleotide. The cleavable moiety may be any cleavable or excisable moiety described elsewhere herein (e.g., uracil, ribonucleotide, etc.). The first adaptor 1761 of the second surface oligonucleotide may comprise one or more coupling moieties (denoted “\hat{ }”) that can permit coupling of the second surface oligonucleotide to another oligonucleotide alternatively or in addition to hybridization. For example, the one or more coupling moieties may comprise a cross-linking reagent (e.g., ^{CNV}K) that crosslinks to a base in the other oligonucleotide. Any other coupling mechanism described elsewhere herein, covalent or noncovalent, may be used. The one or more coupling moieties may comprise any other moiety (e.g., sulfur for S—S bonding, click chemistry reagents, etc.). The coupling may be activated, such as via light treatment or application of one or more stimuli (e.g., heat, light, chemical reagent, etc.). The coupling may be reversible or irreversible. In some cases, the first adaptor 1761 of the second surface oligonucleotide may have the same and/or at least partially overlapping sequence as the first surface oligonucleotide 1751. A library molecule 1771 which is at least partially complementary to the first surface oligonucleotide 1751 may be hybridized to the first surface oligonucleotide and/or the second surface oligonucleotide 1752 (the figure illustrating hybridization with only the first surface oligonucleotide). The library molecule may be a single stranded DNA template comprising insert sequence 1755.

In 1701, the library molecule 1771 may be amplified to generate copies and/or reverse complement copies that are bound, covalently or non-covalently (e.g., hybridized), to the support 1750. For example, during amplification, the first surface oligonucleotide 1751 may be extended using the library molecule 1771 as a template to generate first extended surface molecule 1762. Double-stranded molecules on the support may be subjected to stripping, such as via NaOH treatment, to strip away the library molecule 1771 from the support. In place, extension primer 1764 may be annealed to a 3′ end of the first extended surface molecule and extended using the first extended surface molecule as a template to generate second extended molecule 1763. The second extended molecule 1763 may be denatured from the first extended surface molecule and hybridized to the second surface oligonucleotide 1752. This process may be repeated any number of times to generate multiple first extended surface molecules (e.g., 1762, extended from other first surface oligonucleotides on the support) and multiple second extended molecules (e.g., 1763) hybridized to the first extended surface molecules and/or second surface oligonucleotides. Optionally, extension primer 1764 may comprise a capture entity that may be used to enrich a mixture of supports post-amplification to isolate for positive supports (comprising amplified copies of library molecules). Any capture and capturing mechanism described elsewhere herein may be used, such as biotin and streptavidin binding. Optionally, extension primer 1764 may comprise one or more cleavable moieties disposed 3′ of a capture entity (e.g., biotin) such that the capture entity can be cleaved from the support by cleaving or excising the one or more cleavable moieties, such as after enrichment.

Then, in 1702, the one or coupling moieties (“\hat{ }”) in the second surface oligonucleotide 1752 may be activated to couple the second surface oligonucleotide to the second extended molecule 1763, in addition to hybridization. For example, cross-linking may be activated via light treatment. After coupling, double-stranded molecules on the support may be subjected to stripping, such as via NaOH treatment, to strip away any hybridized second extended molecules (e.g., 1763) that is not otherwise coupled (e.g., cross-linked) to second surface oligonucleotides (e.g., 1752). After stripping, the support 1750 may comprise first extended surface molecules (e.g., 1762) and only those second extended molecules (e.g., 1763) coupled to second surface oligonucleotides (e.g., 1752). The one or more cleavable moieties (“*”) in the second surface oligonucleotide 1752 may be cleaved or otherwise removed to unblock the second surface oligonucleotide, providing an unblocked second primer on the support 1750 that is capable of extending. In 1703, the unblocked second primer may be used as a first sequencing primer and be extended in a sequencing reaction, using the second extended molecule 1763 hybridized to the second surface oligonucleotide 1752 as a template, to generate a first sequencing read comprising a first sequence corresponding to a sequence of the template insert sequence 1755 in the 3′ to 5′ direction. In 1704, a second sequencing primer 1767 may bind to the first extended surface molecule 1762 at or adjacent to the 3′ end and be extended in a sequencing reaction to generate a second sequencing read comprising a second sequence corresponding to a sequence of the template insert sequence 1755 in 5′ to 3′ direction. The first sequencing read and the second sequencing read may be paired and processed to generate a paired read of the template insert 1755.

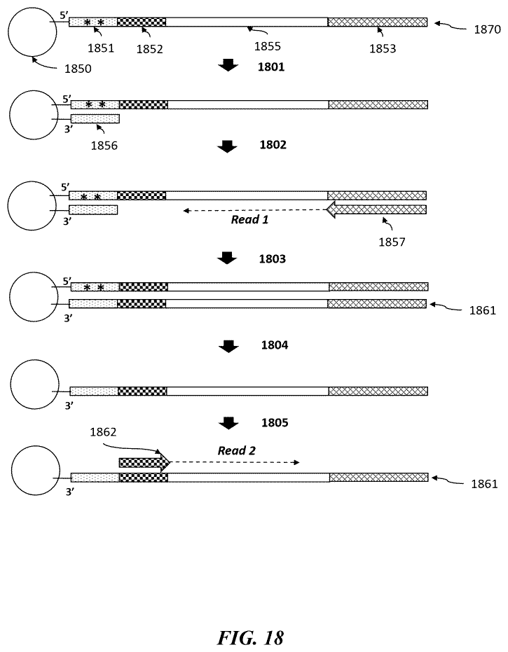

FIG. 18 shows an oligonucleotide molecule 1870 whose 5′ end is immobilized to a support 1850, such as a bead. The oligonucleotide molecule may be covalently or non-covalently bound to the support. The oligonucleotide molecule may comprise, in order of 5′ to 3′, a first adaptor 1851, a second adaptor 1852, a template insert sequence 1855, and a third adaptor 1853. The oligonucleotide molecule may comprise one or more cleavable moieties (denoted “*” in the figure) at or adjacent to a 5′ end, for example in the first adaptor 1851. The cleavable moiety may be any cleavable or excisable moiety described elsewhere herein (e.g., uracil, ribonucleotide, etc.). In 1801, an additional oligonucleotide molecule 1856 may be immobilized to the support at 3′ end. The additional oligonucleotide molecule may be covalently or non-covalently bound to the support via any coupling mechanism described elsewhere herein. For example, in some cases, the additional oligonucleotide molecule may be attached via click chemistry, such as via a first reactive moiety on the support and a second reactive moiety at a 3′ end of the additional oligonucleotide molecule. Example click chemistry pairs are described elsewhere herein (e.g., DBCO-azide pair). The additional oligonucleotide molecule may be at least partially complementary to and hybridized to the first adaptor of the oligonucleotide molecule.

Then, in 1802, a first sequencing primer 1857 may bind at or adjacent to a 3′ end of the oligonucleotide molecule and be extended in a sequencing reaction to generate a first sequencing read comprising a first sequence corresponding to a sequence of the template insert sequence 1855 in 3′ to 5′ direction. The first sequencing primer may be at least partially complementary to and bind to the third adaptor 1853 region. In 1803, the extended molecule may be joined to the additional oligonucleotide molecule 1856 via ligation to generate first extended molecule 1861 that is hybridized to oligonucleotide molecule 1870. In 1804, the one or more cleavable moieties in the oligonucleotide molecule 1870 may be cleaved or excised, and a remainder of the strand digested or otherwise removed (e.g., stripped), thus leaving only the first extended molecule 1861 bound to the support at 3′ end. Optionally, a portion of the oligonucleotide molecule 1870 (closer to 5′ end) may also remain bound to the support (not shown in the figure), such as when the one or more cleavable moieties are 3′ to one or more bases and such base(s) are not digested. In 1805, a second sequencing primer 1862 may bind to the first extended molecule 1861 at or adjacent to a 3′ end and be extended in a sequencing reaction to generate a second sequencing read comprising a second sequence corresponding to a sequence of the template insert sequence 1855 in 5′ to 3′ direction. The second sequencing primer may be at least partially complementary to and bind to a region of the first extended molecule 1861 corresponding to the second adaptor 1852. The first sequencing read and the second sequencing read may be paired and processed to generate a paired read of the template insert 1855.

In some cases, alternatively or in addition to the oligonucleotide molecule comprising one or more cleavable moieties at or adjacent to 5′ end, the oligonucleotide molecule may be synthesized with dUTPs during amplification (not shown in FIG. 18) to generate copies of the oligonucleotide molecules (e.g., 1870) bound to the support 1850, such as during PCR, RPA, ePCR, etc., comprising dUTPs. Such dUTPs may be used to degrade the whole strand in 1804. In some cases, alternatively or in addition to the oligonucleotide molecule comprising one or more cleavable moieties at or adjacent to 5′ end, the oligonucleotide molecule 1870 may be bound to the support 1850 via a cleavable linker, and the cleavable linker may be cleaved in 1804. Examples of cleavable linkers include reversible crosslinking bonds, disulfide bonds (e.g., cleaved via reducing agent), etc.

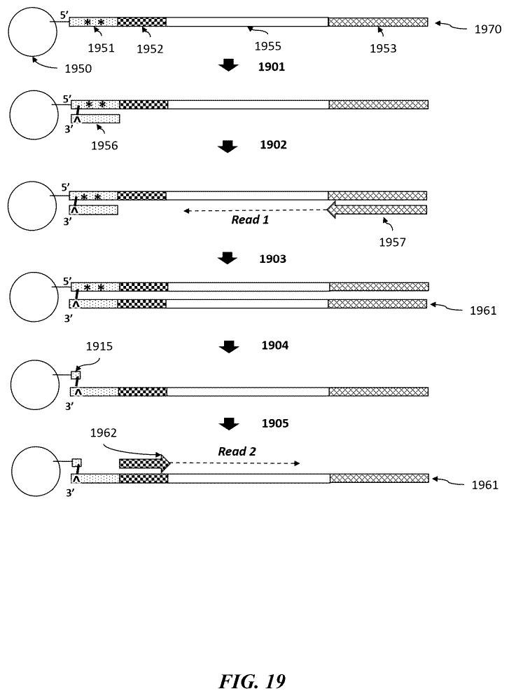

FIG. 19 shows an oligonucleotide molecule 1970 whose 5′ end is immobilized to a support 1950, such as a bead. The oligonucleotide molecule may be covalently or non-covalently bound to the support. The oligonucleotide molecule may comprise, in order of 5′ to 3′, a first adaptor 1951, a second adaptor 1952, a template insert sequence 1955, and a third adaptor 1953. The oligonucleotide molecule may comprise one or more cleavable moieties (denoted “*” in the figure) at or adjacent to a 5′ end, for example in the first adaptor 1951. The cleavable moiety may be any cleavable or excisable moiety described elsewhere herein (e.g., uracil, ribonucleotide, etc.). In 1901, an additional oligonucleotide molecule 1956 may be hybridized to at least a portion of the first adaptor 1951 region of the oligonucleotide molecule. Additional oligonucleotide molecule 1956 may be 5′ phosphorylated (e.g., to enable ligation with another molecule). Alternatively or in addition, additional oligonucleotide molecule 1956 may comprise a 3′ blocking moiety (e.g., 3′ end of 1956 may comprise a dideoxy nucleotide). Any suitable blocking or capping mechanism described elsewhere herein may be used. The additional oligonucleotide molecule may be at least partially complementary to the first adaptor. The additional oligonucleotide molecule and the oligonucleotide may be coupled in addition to the hybridization, such as via crosslinking, click chemistry, or any other coupling mechanism described elsewhere herein. The coupling may be covalent or non-covalent. In the illustration, as an example, the additional oligonucleotide molecule comprises one or more coupling moieties (denoted “\hat{ }” in the figure). The one or more coupling moieties may comprise a cross-linking reagent (e.g., ^{CNV}K) that crosslinks to a base in the second adaptor. The one or more coupling moieties may comprise any other moiety (e.g., sulfur for S—S bonding, click chemistry reagents, etc.). The coupling may be activated, such as via light treatment or application of one or more stimuli (e.g., heat, light, chemical reagent, etc.). The coupling may be reversible or irreversible. The additional oligonucleotide molecule may be coupled to the oligonucleotide molecule at a coupling site in the oligonucleotide molecule that is 5′ to the one or more cleavable moieties in the oligonucleotide molecule and at 3′ end of the additional oligonucleotide molecule.

Then, in 1902, a first sequencing primer 1957 may bind at or adjacent to a 3′ end of the oligonucleotide molecule and be extended in a sequencing reaction to generate a first sequencing read comprising a first sequence corresponding to a sequence of the template insert sequence 1955 in 3′ to 5′ direction. The first sequencing primer may be at least partially complementary to and bind to the third adaptor 1953 region. In some cases, where additional oligonucleotide molecule 1956 is not blocked at the 3′ end, step 1902 may further comprise blocking or capping 3′ end of 1956. For example, 3′ end of 1956 may be phosphorylated.

In 1903, the extended molecule may be joined to the additional oligonucleotide molecule 1956 via ligation to generate first extended molecule 1961 that is hybridized to oligonucleotide molecule 1970. In 1904, the one or more cleavable moieties in the oligonucleotide molecule may be cleaved or excised, and a remainder of the strand that is disposed 3′ of the one or more cleavable moieties may be digested or otherwise removed (e.g., stripped), leaving only a portion 1915 of the oligonucleotide molecule (closer to 5′ end) bound to the support 1950. In some cases, the one or more cleavable moieties may comprise uracil-based cleavage sites, and these sites may be cleaved using a mix of Uracil-DNA Glycosylase (UDG) and Endonuclease VIII enzymes. The UDG may catalyze the hydrolysis of the N-glycosidic bond from deoxyuridine to release uracil, and Endonuclease VIII may cleave the DNA phosphodiester backbone at AP, creating a 1-nucleotide DNA gap with 5′ and 3′ phosphate termini. That is, use of the UDG/Endo VIII enzyme combination results in a blocked 3′ end of the portion 1915 (e.g., 3′ phosphorylation). In some cases, where the cleavage may not result in a blocked 3′ end of 1915, step 1904 may further comprise blocking or capping 1915. For example, 3′ end of 1915 may be phosphorylated.

The portion 1915 may comprise the coupling site (e.g., base and/or moiety) that is coupled to the additional oligonucleotide molecule 1956 of the first extended molecule 1961. The portion 1915 may comprise a single base. The portion 1915 may comprise a plurality of bases (see e.g., FIG. 19, where 3′ oligonucleotide of 1915 is coupled to additional oligonucleotide 1956). The portion 1915 may not comprise any bases, but for example comprise one or more moieties that act as the coupling site. Thus, the first extended molecule 1961 may remain bound to the support, covalently or non-covalently, via the portion 1915. In some cases, at least a part of the portion 1915 may remain hybridized to the first extended molecule 1961. In other cases, the portion 1915 may not be hybridized to the first extended molecule 1961.

In 1905, a second sequencing primer 1962 may bind to the first extended molecule 1961 at or adjacent to a 3′ end and be extended in a sequencing reaction to generate a second sequencing read comprising a second sequence corresponding to a sequence of the template insert sequence 1955 in 5′ to 3′ direction. The second sequencing primer may be at least partially complementary to and bind to a region of the first extended molecule 1961 corresponding to the second adaptor 1952. The first sequencing read and the second sequencing read may be paired and processed to generate a paired read of the template insert 1955.

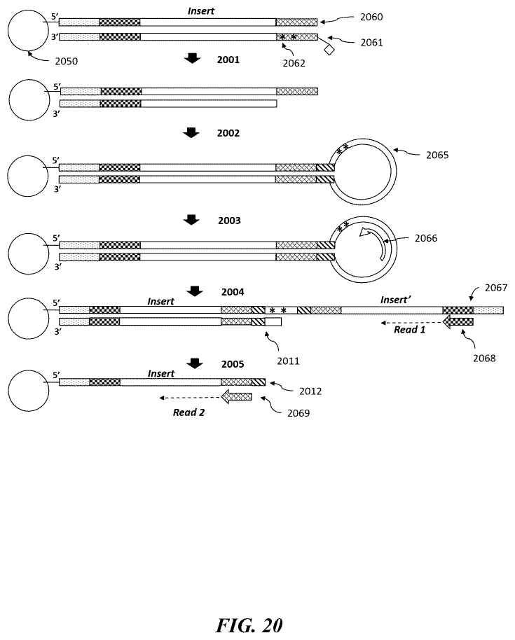

FIG. 20 shows an oligonucleotide molecule 2060 whose 5′ end is immobilized to a support 2050, such as a bead. The oligonucleotide molecule may be covalently or non-covalently bound to the support. An extension primer 2062 may be annealed to a 3′ end of the oligonucleotide molecule and extended using the oligonucleotide molecule as a template to generate an extended molecule 2061. Optionally, extension primer 2062 may comprise a capture entity that may be used to enrich a mixture of supports post-amplification to isolate for positive supports (comprising amplified copies of library molecules). Any capture and capturing mechanism described elsewhere herein may be used, such as biotin and streptavidin binding. Extension primer 2062 may comprise one or more cleavable moieties at or adjacent to a 5′ end. Where there is a capture entity, the one or more cleavable moieties may be disposed 3′ of a capture entity (e.g., biotin) such that the capture entity can be cleaved from the support by cleaving or excising the one or more cleavable moieties, such as after enrichment. In 2001, the one or more cleavable moieties may be cleaved to remove a 5′ portion of the extended molecule 2061, leaving a double-stranded molecule with a single-stranded overhang bound to the support, the single-stranded overhang corresponding to a 3′ portion of the oligonucleotide molecule 2060.

In 2002, a hairpin adaptor 2065 may be ligated to the double-stranded molecule on the support. The hairpin adaptor may comprise a stem portion and a loop portion. The stem portion may comprise a double-stranded portion and a single-stranded overhang, which single-stranded overhang is complementary to and binds to the single-stranded overhang of the double-stranded molecule bound to the support. The loop portion may comprise one or more cleavable moieties (denoted “*”). In 2003, a loop primer 2066 may be annealed to the loop portion of the hairpin adaptor and extended with a strand displacing polymerase, to generate a second extended molecule 2011 hybridized to a 5′ portion of the oligonucleotide molecule 2060, opening the former double-stranded molecule to a long strand 2067. In 2004, a first sequencing primer may bind to a 3′ end of the long strand 2067 and be extended in a sequencing reaction to generate a first sequencing read comprising a first sequence corresponding to a sequence of the template insert sequence in 5′ to 3′ direction. In 2005, the one or more cleavable moieties introduced in the loop portion, now in the middle of the long strand 2067, may be cleaved to remove 3′ portion of the long strand, generating a short strand 2012. Double-stranded molecules on the support may be subjected to stripping, such as via NaOH treatment, to remove hybridized strands including the second extended molecule 2011. A second sequencing primer 2069 may be extended in a sequencing reaction to generate a second sequencing read comprising a second sequence corresponding to a sequence of the template insert sequence in 3′ to 5′ direction. The first sequencing read and the second sequencing read may be paired and processed to generate a paired read of the template insert.

Any operation described herein may be performed while the support is in solution or immobilized to another surface, e.g., substrate surface. Any operation described herein may be performed in a sequencing instrument. Any operation described herein may be performed in an amplification instrument. Any operation described herein may be performed in bulk solution (e.g., multiple supports and reagents in fluid communication) or in partitions (e.g., a partition of a plurality of partitions, such as droplets or wells, containing at most one support in an isolated reaction environment). In some cases, at least a first sequencing read generation step and all subsequent steps (e.g., strand displacement, cleaving, second sequencing read generation step) may be performed while the support is immobilized to a substrate surface, such as in a sequencing instrument.

While each of the workflows of FIGS. 12-20 illustrate and exemplify generation of a first sequencing read from a first single strand and a second sequencing read from a second single strand, it will be appreciated that any sequencing read may be generated from multiple strands (e.g., copies) in a colony. For example, one or more operations described herein may generate a colony of copies of a first strand used to generate a read corresponding to an insert in a 5′ to 3′ direction and a colony of copies of a second strand used to generate a read corresponding to an insert in a 3′ to 5′ direction, and sequencing reactions may collect cumulative signals from each colony. Thus, it will be appreciated that for each support described in any of the workflows, duplicates or multiple copies of each molecule may exist and be likewise related to the support as the one(s) exemplified and each operation described may be performed in parallel and/or simultaneously for each of the duplicates or multiple copies related to the support.

A sequencing read may degrade in quality as the length of the read increases, such as due to increasing sequencing errors. Thus, even within a single sequencing read, the earlier called bases may have a higher quality or accuracy than the later called bases. Beneficially, for all paired end methods described herein, a template insert or sequence(s) corresponding thereto (an identical sequence, a reverse complement sequence) is read from each end, ensuring that there is a high quality read at each end of the template insert. In some cases, the first sequencing read and the second sequencing read may partially or completely overlap a region of the template insert. In some cases, the first sequencing read and the second sequencing read may not overlap any region of the template insert.

Sequencing Methods with Incorporable and Non-Incorporable Nucleotides

During sequencing by synthesis, nucleotides of known canonical base types are incorporated into extending primers (e.g., primers hybridized to template nucleic acid molecules) and the sequence of template nucleic acid molecules is determined based on identifying which canonical base type has been incorporated. Accurate sequencing depends upon incorporation of an appropriate nucleotide (e.g., a nucleotide in canonical Watson-Crick base pairing with a template nucleic acid molecule) in each sequencing step and a high confidence in the detection of what nucleotide base type has been incorporated. There are multiple potential sources of error during sequencing. These include: misincorporation (e.g., incorrect nucleotide base pairing to template molecule), precision of signal detection (e.g., including signal-to-noise-ratio, signal intensity, resolution, and crosstalk), and lagging incorporation or precocious incorporation (i.e., resulting in phasing of signal). Sequencing by synthesis (SBS), which is based on the hybridization and incorporation of terminated or unterminated nucleotide(s) within a single step is particularly prone to phasing issues (e.g., non-synchronous incorporation events within a sequencing colony) since the incorporated nucleotides are typically labeled with detectable moieties that are chemically cleaved each step (where cleavage results in scars that can inhibit further incorporation). See e.g., Fuller et al. (2009) The challenges of sequencing by synthesis. Nat. Biotech. 27 (11), 1013-1023. Beneficially, by using separate sequencing steps for detection of labeled nucleotides and incorporation of unlabeled nucleotides, e.g., as is possible with Sequencing by binding (SBB), labeling scars can be eliminated, and phasing can be reduced.

### Non-Incorporable Nucleotides

In some cases, a non-incorporable nucleotide may be modified (e.g., chemically modified), relative to a native nucleotide. In some cases, a non-incorporable nucleotide may be chemically modified analogs of the native nucleotide that cannot be used as a substrate nucleotide by a polymerase in a nucleic acid polymerization reaction. For example, the chemically modified non-incorporable nucleotide may not be incorporated enzymatically into the 3′ end of an extending primer molecule hybridized to a template nucleic acid molecule.

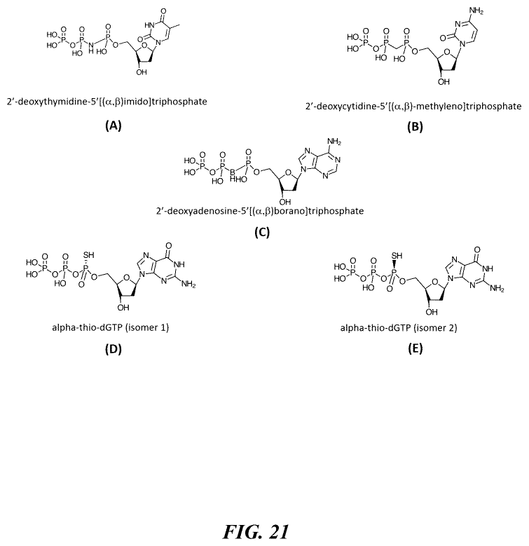

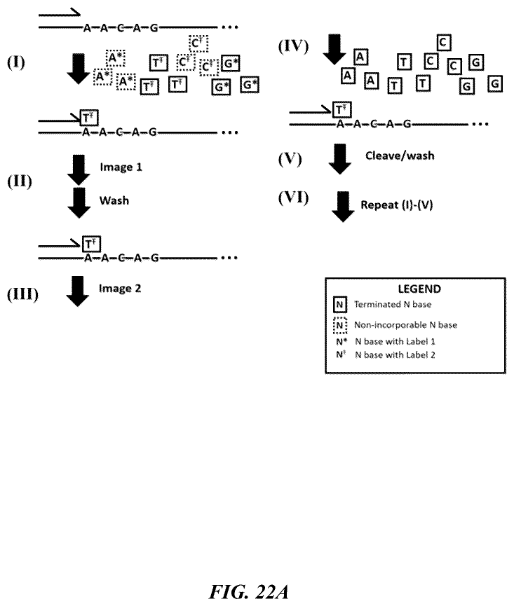

In some cases, a non-incorporable nucleotide may comprise a deoxyribonucleoside triphosphate (dNTP). In some cases, a non-incorporable nucleotide may comprise alpha thio dNTPs, alpha methylene dNTPs, borano dNTPs, alpha imino dNTPs, beta thio dNTPs, beta methylene dNTPs, beta imino dNTPs, 5′-(beta, gamma-imido) dNTPs, 3′ azidomethyl dNTPs, 5′-(alpha, beta-thio) dNTPs, or a combination thereof. In some cases, a non-incorporable nucleotide may comprise the chemicals shown in FIG. 21 or variations thereof. Panel (A) illustrates 2′-deoxythymidine-5′ [(α,β)imido]triphosphate. Panel (B) illustrates 2′-deoxycytidine-5′ [(α,β)-methyleno]triphosphate. Panel (C) illustrates 2′-deoxyadenosine-5′ [(α,β)borano]triphosphate. Panel (D) illustrates an isomer of alpha-thio-dGTP. Panel (E) illustrates another isomer of alpha-thio-dGTP.

In some cases, a non-incorporable nucleotide may comprise a D-nucleotide, an L-nucleotide, or a combination thereof.

In some cases, a non-incorporable nucleotide may be hydrolysable, e.g., by a polymerase. In some cases, a non-incorporable nucleotide may be non-hydrolysable, e.g., by a polymerase. A non-hydrolysable nucleotide may comprise alpha thio deoxyribonucleoside triphosphates (dNTPs), alpha methylene dNTPs, borano dNTPs, alpha imino dNTPs, beta thio dNTPs, beta methylene dNTPs, beta imino dNTPs, or a combination thereof. In some cases, a non-hydrolysable nucleotide may comprise a dideoxynucleoside triphosphate (ddNTPs).

In some cases, a non-incorporable nucleotide may be modified to increase a binding affinity of the non-incorporable nucleotide, to increase the efficiency. For example, the base and/or the sugar of the nucleotide may be modified while keeping the triphosphate group non-cleavable. In an example, the methyl at the 5 position of thymidine or the hydrogen atom at the 5 position of deoxycytidine is substituted by a C-triple bond-C—CH₂—OH group.

A nucleotide mixture comprising non-incorporable nucleotides may comprise non-incorporable nucleotides of a single type (e.g., alpha thio dNTPs). A nucleotide mixture comprising non-incorporable nucleotides may comprise non-incorporable nucleotides of multiple types.

In some cases, a polymerase may be configured to prevent or inhibit the mis-incorporation of incorporable nucleotide. In some cases, the polymerase may comprise a mutation. Such a mutation may render an otherwise incorporable nucleotide non-incorporable. A mutation may comprise a nucleotide (or the resultant amino acid) substitution, addition, deletion, or a combination thereof.

Also provided herein are non-incorporable nucleotide groups which may be used in addition to or alternatively to individual non-incorporable nucleotides or a plurality thereof. A non-incorporable nucleotide group may comprise a multimeric or dendrimeric structure which comprises a core structure covalently linked to a plurality of non-incorporable nucleotide triphosphates. For example, a core structure may be linked to at least 2, 3, 4, 5, 6, 7, 8, 9, 10, 20, 30, 40, 50 or more non-incorporable nucleotide triphosphates. Such non-incorporable nucleotide groups may significantly increase binding affinity by creating a higher local concentration (when using a plurality of non-incorporable nucleotide triphosphates of a single base type). In some cases, a single non-incorporable nucleotide group may comprise a plurality of non-incorporable nucleotide triphosphates of a single base type. In some cases, a single non-incorporable nucleotide group may comprise a plurality of non-incorporable nucleotide triphosphates of multiple base types (e.g., 1, 2, 3 base types).

In some cases, a non-incorporable nucleotide may further comprise a terminating moiety.

### One Labeled Nucleotide Flow, Two Imaging Steps

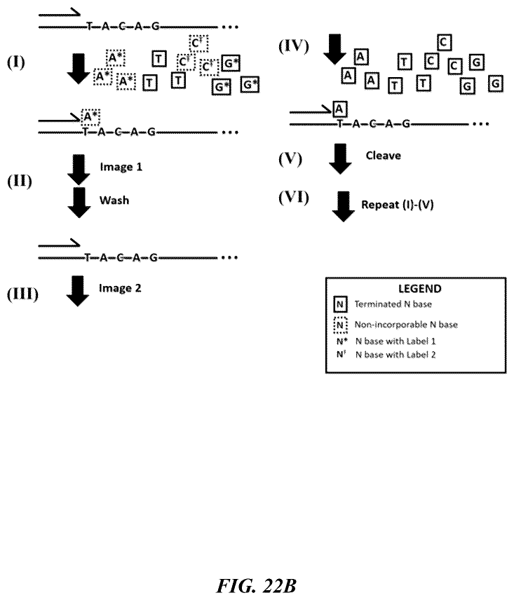

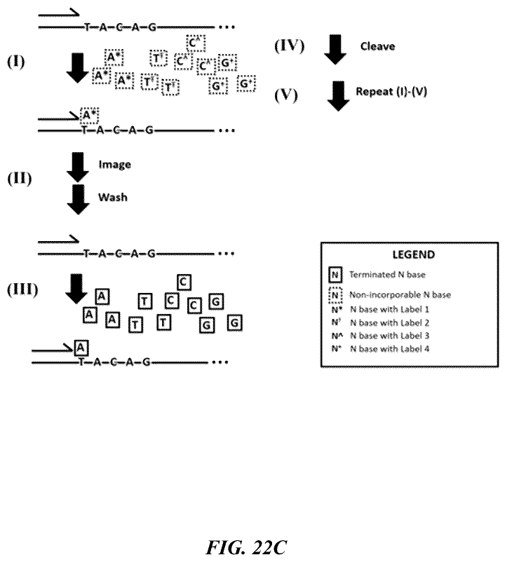

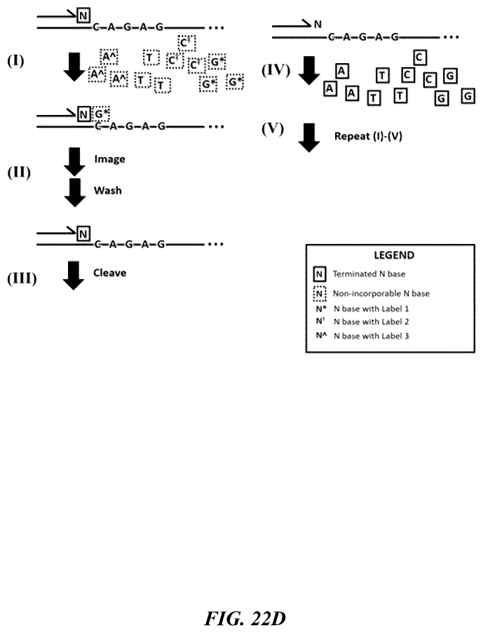

FIGS. 22A and 22B illustrate exemplary schemes for combined reversibly terminated and non-incorporable (e.g., transiently bound) nucleotide sequencing. A template is hybridized to a growing strand (e.g., an extending primer) which is ready to extend through a portion in the template. The growing strand is unblocked (e.g., unterminated). The growing strand may incorporate the terminated bases, but the non-incorporable cases will only be capable of binding to the polymerase and the template. In step (I), the labeled nucleotide flow, the growing strand is contacted with a first nucleotide mixture comprising at least some labeled nucleotides. In FIG. 22A the first nucleotide mixture comprises labeled terminated and labeled non-incorporable bases of all four canonical base types. The growing strand incorporates one reversibly terminated T, where the nucleotide base is labeled with a second label type. The first nucleotide mixture comprises non-incorporable A bases coupled to a first type of detectable label, non-incorporable C bases coupled to a second type of detectable label, reversibly terminated T bases coupled to the second type of detectable label, and reversibly-terminated G bases coupled to the second type of detectable label. As described elsewhere herein, different combinations of reversibly terminated and labeled bases may be used (e.g., reversibly terminated A with first type of label, reversibly terminated T with second type of label, non-incorporable C with first type of label, and non-incorporable G with second type of label). In some cases, only three nucleotide base types may be used (e.g., reversibly terminated A with first type of label, non-incorporable C with second type of label, and non-incorporable G with first type of label—e.g., where T bases are lacking). In another example, as illustrated in FIG. 22B, all four canonical base types may be present where three base types are labeled, and one base type is unlabeled. In FIG. 22B, the first nucleotide mixture comprises non-incorporable A with a first label type, non-incorporable C with a second label type, reversibly terminated G with the first label type, and reversibly terminated T without a label. It will be understood that other combinations of non-incorporable and reversibly terminated base types and other combinations of base types labeled with a first label type, a second label type, and/or no label may be used. In step (II), in FIGS. 22A and 22B, a first imaging is performed to collect first signals (e.g., T). The first imaging step will detect signals indicative of incorporation of reversibly terminated bases or binding of non-incorporable bases. The first nucleotide mixture is then washed away. The washing will remove nucleotides that are merely bound to the template (e.g., the non-incorporable nucleotides) and will not remove nucleotides incorporated into the growing strand (e.g., the reversibly terminated nucleotides). In step (III), in FIGS. 22A and 22B, a second imaging is performed to collect second signal or lack thereof. The second imaging step will detect signals indicative of binding of the reversibly terminated bases as those will not be removed by the washing. In cases where one base type is wholly unlabeled, no signal detected in first and second imaging steps will indicate incorporation/binding of nucleotides of that one base type. A comparison of the first signal and the second signal will reveal which nucleotide base type has been incorporated/bound, thereby indicating the sequence of the template. In step (IV), in FIGS. 22A and 22B, the unlabeled nucleotide flow (e.g., a dark extension step), the growing strand is contacted with a second nucleotide mixture comprising unlabeled terminated bases of all four canonical base types to extend the growing strand by a single base. In step (V), in FIGS. 22A and 22B, reversible terminators are cleaved, and labeling moieties are cleaved as required (e.g., from reversibly terminated nucleotides incorporated from the first nucleotide flow). Only a single base of the template sequence is determined in each set of steps (I)-(V). In step (VI), in FIGS. 22A and 22B, steps (I)-(V) may be repeated any number of times to determine a sequence of a template molecule.

The first nucleotide mixture may comprise 100% labeled nucleotides. In some cases, for each base type, a ratio of labeled to unlabeled nucleotides may be used, as described elsewhere wherein. 100% labeling may be required in single-molecule sequencing, and in contrast partial labeling may be sufficient in colony-based sequencing. Steps (I)-(V) may be repeated, for example, at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 600, 700, 800, 900, 1000, 2000, 3000, 4000, 5000, 10,000 or more times.

Advantageously, in the schemes illustrated FIGS. 22A and 22B, at most only eight types of nucleotide analogs are required (e.g., across the first nucleotide flow and the second nucleotide flow). Only a single flow with labeled nucleotides is required (e.g., first nucleotide flow). In addition, advantageously there may be no scars (e.g., as a result of cleaving label linkers) on the bases that are non-incorporable (e.g., A and C bases in both FIGS. 22A and 22B), and (as illustrated in FIG. 22B) there may be no scar for a reversibly terminated unlabeled base type (e.g., T).

### One Labeled Nucleotide Flow, One Imaging Step

FIGS. 22C and 22D illustrate exemplary schemes for combined reversibly terminated and non-incorporable (e.g., transiently bound) nucleotide sequencing. A template is hybridized to a growing strand (e.g., an extending primer) which is ready to extend through a portion in the template. In FIG. 22C, The growing strand is unblocked (e.g., unterminated). In FIG. 22D, The growing strand is blocked (e.g., reversibly terminated). The growing strand in each case may bind to non-incorporable nucleotides, but the blocked growing strand in FIG. 22D is not capable of incorporating any nucleotides without cleavage of the blocking moiety. In step (I), the labeled nucleotide flow, the growing strand is contacted with a first nucleotide mixture comprising at least some labeled nucleotides. In FIG. 22C the first nucleotide mixture comprises non-incorporable A with a first label type, non-incorporable T with a second label type, non-incorporable C with a third label type, and non-incorporable G with a fourth label type. A labeled non-incorporable A base is bound to the template strand. It will be understood that different combinations of label types and unlabeled non-incorporable nucleotides may be used. In some cases, only three nucleotide base types may be used (e.g., A bases may be lacking). In another example, as illustrated in FIG. 22D, all four canonical base types may be present in non-incorporable form where three base types are labeled, and one base type is unlabeled. A labeled, non-incorporable G is bound to the template molecule. In FIG. 22D, the first nucleotide mixture comprises non-incorporable G with a first label type, non-incorporable C with a second label type, non-incorporable A with a third label type, and non-incorporable T without a label. It will be understood that other combinations of base types labeled with a first label type, a second label type, a third label type, a fourth label type and/or no label may be used. In step (II), in FIGS. 22C and 22D, an imaging is performed to collect signals (e.g., indicative of A and G, respectively). The imaging step will detect signals indicative of binding of non-incorporable, labeled bases. The first nucleotide mixture is then washed away. The washing will remove nucleotides that are merely bound to the template (e.g., the non-incorporable nucleotides). In step (III), in FIG. 22C, the growing strand is contacted with a second nucleotide mixture comprising unlabeled terminated bases of all four canonical base types to extend the growing strand by a single base (e.g., an A). In FIG. 22D, the growing strand is blocked, so in step (III), the blocking moiety (e.g., a reversible terminator) is cleaved. In step (IV), in FIG. 22C, reversible terminators are cleaved. In step (IV), in FIG. 22D, the growing strand is contacted with a second nucleotide mixture comprising unlabeled terminated bases of all four canonical base types to extend the growing strand by a single base (e.g., a G). Only a single base of the template sequence is determined in each set of steps (I)-(IV). In step (V), in FIGS. 22C and 22D, steps (I)-(IV) may be repeated any number of times to determine a sequence of a template molecule.

The first nucleotide mixture may comprise 100% labeled nucleotides. In some cases, for each base type, a ratio of labeled to unlabeled nucleotides may be used, as described elsewhere wherein. 100% labeling may be required in single-molecule sequencing, and in contrast partial labeling may be sufficient in colony-based sequencing. Steps (I)-(V) may be repeated, for example, at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 600, 700, 800, 900, 1000, 2000, 3000, 4000, 5000, 10,000 or more times.

Advantageously, in the schemes illustrated FIGS. 22C and 22D, at most only eight types of nucleotide analogs are required (e.g., across the first nucleotide flow and the second nucleotide flow). Only a single flow with labeled nucleotides is required (e.g., first nucleotide flow). In addition, advantageously there may be no scars (e.g., as a result of cleaving label linkers) on the incorporated nucleotides.

A method (e.g., for sequencing template nucleic acid molecules) may comprise: (a) providing a plurality of template molecules, wherein the template molecules comprise primers hybridized thereto, (b) contacting the plurality of template molecules with a reaction mixture comprising a plurality of nucleotides under conditions sufficient for one or more nucleotides to be incorporated into one or more primers hybridized to template molecules and for one or more nucleotides to be transiently bound to one or more template molecules, and (c) detecting a signal, wherein the signal indicates the presence or absence of incorporated or transiently bound nucleotides.

After the signal is detected, the plurality of template molecules may be washed to remove transiently bound nucleotides. A washing step may additionally remove polymerase and other components of the reaction mixture. Nucleotides that have been incorporated into extending primers hybridized to template molecules may not be removed by washing. Incorporated nucleotides comprise covalent bonds and effectively become part of extending primers. After the removal of transiently bound nucleotides, additional signal may be detected. Additional signal detected in this second imaging/detection step may indicate the presence or absence of incorporated nucleotides.

Another method (e.g., for sequencing template nucleic acid molecules) may comprise: (a) providing a plurality of template molecules with primers hybridized thereto, (b) contacting the plurality of template molecules with a reaction mixture, wherein the reaction mixture comprises terminated and unterminated nucleotides, and wherein primers of a first subset of the plurality of template molecules incorporate a terminated nucleotide and a second subset of the plurality of template molecules transiently bind to an unterminated nucleotide, (c) detecting signal, wherein the signal indicates the presence or absence of incorporated and transiently bound nucleotides, (d) removing transiently bound nucleotides from the second subset of the plurality of template molecules, and (e) detecting signal, wherein the signal indicates the presence or absence of incorporated nucleotides.

Steps (b)-(e) may be repeated any number of times with the same or different reaction mixtures to determine the sequence of template molecules in the plurality of template molecules.

The plurality of template molecules may comprise substantially identical molecules (e.g., an amplified colony). A substantially identical colony of template molecules may be processed simultaneously to amplify and/or average out signals that are detected from incorporated and/or transiently bound nucleotides. These signals are indicative of incorporation into extending primers, binding to template molecules, or lack thereof. The plurality of template molecules may comprise a plurality of non-identical template molecules. The plurality of template molecules may be amplified. The plurality of template molecules may be un-amplified (e.g., for single-molecule sequencing).

The reaction mixture may further comprise a polymerase. The polymerase may enable incorporation of nucleotides, transient binding of nucleotides, or both. The polymerase may enable incorporation of terminated nucleotides, transient binding of unterminated nucleotides, or both. In some cases, the reaction mixture may further comprise a first polymerase for the incorporation of terminated nucleotides and a second polymerase for the transient binding of unterminated nucleotides. In some cases, unterminated nucleotides are non-incorporable nucleotides (e.g., where the nucleotides comprise nucleotide analogs that are modified e.g., to be unable to form a phosphodiester bond). By way of example, non-incorporable nucleotides may be dideoxynucleotides (ddNTPs) that lack 3′ OH required for forming a phosphodiester bond. In some cases, terminated nucleotides are reversibly terminated (e.g., where the nucleotides comprise a reversibly terminating moiety as described elsewhere herein).

A mixture of nucleotides may comprise a plurality of canonical nucleotide base types (e.g., 2, 3, 4, 5 base types). The nucleotide mixture (e.g., a reaction mixture) may comprise a first nucleotide of a first canonical base type and a second nucleotide of a second canonical base type different from said first canonical base type. The first nucleotide may be an incorporable nucleotide. The second nucleotide may be a non-incorporable nucleotide. The nucleotide mixture may further comprise a third nucleotide of a third canonical base type different from the first and second canonical base types. The third nucleotide may be a non-incorporable or incorporable nucleotide. The nucleotide mixture may further comprise a fourth nucleotide of a fourth canonical base type different from the first, second, and third canonical base types. The fourth nucleotide may be a non-incorporable or incorporable nucleotide. Any of the nucleotides described herein, e.g., the first, second, third, or fourth nucleotide or some or all, may be terminated. Any of the nucleotides described herein, e.g., the first, second, third, or fourth nucleotide or some or all, may be non-terminated. In some cases, the nucleotide mixture may comprise only terminated nucleotides, only non-terminated nucleotides, or a combination of terminated and non-terminated nucleotides. In some cases, the nucleotide mixture may comprise only incorporable, terminated nucleotides, only non-incorporable nucleotides, or a combination of incorporable, terminated nucleotides and non-incorporable nucleotides. In some cases, at most one nucleotide type in the nucleotide mixture is non-incorporable. In some cases, at most one nucleotide type in the nucleotide mixture is reversibly terminated. In some cases, all but one nucleotide type in the nucleotide mixture is non-incorporable. In some cases, all but one nucleotide type in the nucleotide mixture is reversibly terminated. Nucleotides in the reaction mixture may be labeled or unlabeled. Alternatively, or in addition, nucleotides in the reaction mixture may be terminated or non-incorporable. Extending primer molecules are expected to incorporate the incorporable first nucleotide if the nucleotide base opposite the incorporation site is complementary to the first canonical base type. If an incorporable nucleotide complementary to the incorporation site is not present in a reaction mixture, a non-incorporable nucleotide may bind to the incorporation site base.

By way of example, the reaction mixture may comprise nucleotides of a first base type comprising a first type of label, nucleotides of a second base type comprising a second type of label, nucleotides of a third base type comprising the first type of label, and a fourth base type comprising the second type of label. In another example, the reaction mixture may comprise nucleotides of a first base type comprising a first type of label, nucleotides of a second base type comprising a second type of label, nucleotides of a third base type comprising the first type of label, and nucleotides of a fourth base type that are unlabeled. In some cases, nucleotides of the first and second base types are reversibly terminated. Nucleotides of the third and fourth base types may be non-incorporable. Alternatively, nucleotides of the first, second, and fourth base types may be reversibly terminated and nucleotides of the third base type may be non-incorporable.

In sequencing methods comprising two detecting steps, the methods may further comprise, after detecting (e) the additional signal, contacting the plurality of template molecules with an additional reaction mixture, where the additional reaction mixture comprises terminated nucleotides. By incorporating reversibly terminated nucleotides into extending primers, these primers are advanced by a single nucleotide base along the template molecules. For template molecules where the extending primers have already incorporated a reversibly terminated nucleotide in a previous step, no additional nucleotide(s) may be incorporated (e.g., because the reversible terminator moiety prevents the formation of a further phosphodiester bond). For template molecules where the extending primers have not incorporated a reversibly terminated nucleotide in a previous step (e.g., extending primers where the template molecules have been transiently bound to non-incorporable nucleotides in a previous step), an unlabeled, reversibly terminated nucleotide may be incorporated.

In some cases, after incorporation of reversibly terminated nucleotides, the plurality of template molecules may be subjected to conditions sufficient to remove (e.g., cleave) terminating moieties and/or labeling moieties (e.g., labeling moieties from labeled, terminated nucleotides incorporated in either of the labeled nucleotide flows).

The above steps, or subsets thereof, may be repeated as appropriate any number of times to determine the sequence of template molecules.

In cases where the reaction mixture(s) comprise nucleotides with two or fewer types of labels (e.g., red and green fluorescent dyes), multiple imaging steps may be required to decode what nucleotide base type has been incorporated into/transiently bound to a template molecule. In cases where the reaction mixture(s) comprise nucleotides with three or more types of labels (e.g., red, blue, and yellow fluorescent dyes), a single imaging step may be sufficient to determine what nucleotide base type has been incorporated into/transiently bound to a template molecule.

In a non-limiting example, a reaction mixture comprises the four canonical nucleotide base types: Aₓ¹, Tₓ², G_{N}, and C_{N}¹, where x indicates a reversible terminator moiety, N indicates a non-incorporable modification, 1 indicates a first detectable moiety, and 2 indicates a second detectable moiety different from the first detectable moiety. Imaging results for a template molecule comprising the sequence AGCCT are summarized in Table 1 below.

In a first sequencing flow, a Tₓ² nucleotide will bind to the template molecule and be incorporated into an extending sequencing primer hybridized to the template. A first imaging step will detect signal for a second detectable moiety. After a wash step, a second imaging step will detect signal for the second detectable moiety. Subsequently, the terminating moiety and the second cleavable moiety will be cleaved from the incorporated T.

In a second sequencing flow, a C_{N}¹ nucleotide will bind to the template molecule. A first imaging step will detect signal for a first detectable moiety. After a wash step, the bound C nucleotide will be removed (i.e., as the C is non-incorporable, any binding will be transient, e.g., temporarily stabilized by a DNA polymerase in complex with the template molecule). A second imaging step will not detect any signal. Subsequently a terminated C nucleotide (Cₓ) will be incorporated into the extending sequencing primer, and the terminating moiety will subsequently be cleaved.

In each of a third and a fourth sequencing flows, a G_{N} nucleotide will bind to the template molecule. First and second imaging steps will not detect any signals. Sequentially, a terminated G nucleotide (Gₓ) will be incorporated into the extending sequencing primer for each of the interrogated loci in the template molecule, and the terminating moieties will be cleaved.

In a fifth sequencing flow, a Aₓ¹ nucleotide will bind to the template molecule and be incorporated into an extending sequencing primer hybridized to the template. A first imaging step will detect signal for a second detectable moiety. After a wash step, a second imaging step will detect signal for the second detectable moiety. Subsequently, the terminating moiety and the second cleavable moiety will be cleaved from the incorporated T.

In each of these five sequencing flows, the template molecule is contacted with a reaction mix comprising Aₓ¹, Tₓ², G_{N}, and C_{N}¹. In each flow, a nucleotide base with sequence complementarity to the available base in the template molecule, will bind to the template molecule. In cases where the bound nucleotide comprises a terminating moiety and does not comprise an incorporation blocker (e.g., a non-incorporation modification), the bound nucleotide will be incorporated into the primer hybridized to the template molecule (e.g., where the incorporation is catalyzed by a DNA polymerase). In cases where the bound nucleotide comprises an incorporation blocker, the bound nucleotide will be only transiently bound to the template molecule (e.g., where the binding is mediated by a DNA polymerase bound to the primer/template molecule complex). As illustrated in Table 1, the pattern of signals detected for each sequencing flow indicates which nucleotide base is present at the available locus in the template molecule.

It will be understood that other combinations of reversibly terminated and non-incorporable nucleotides may be used. Alternatively, or in addition, different combinations of nucleotides with detectable moieties may be used.

### Computer Systems

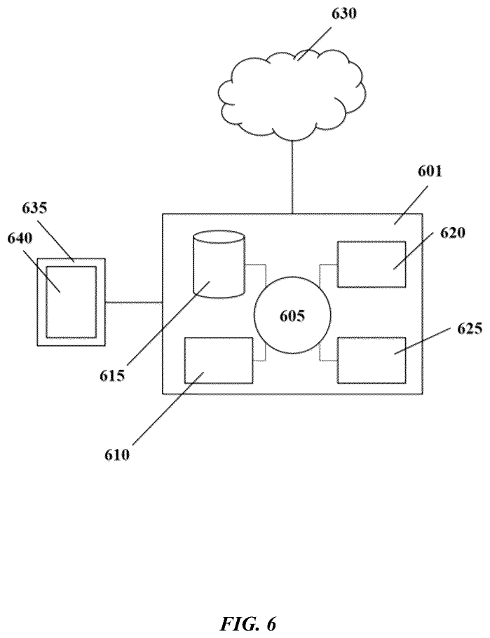

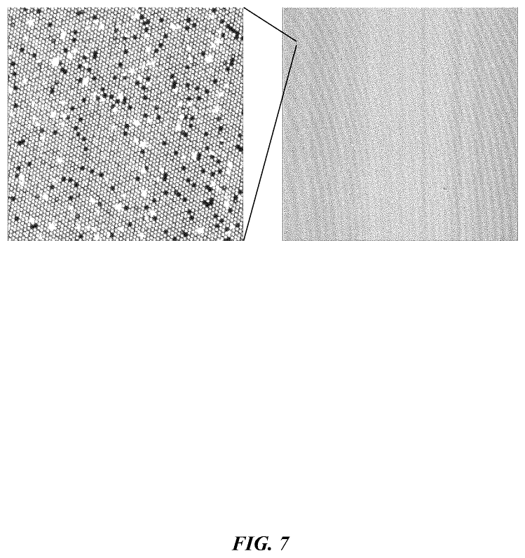

The present disclosure provides computer control systems that are programmed to implement methods of the disclosure. FIG. 6 shows a computer system 601 that is programmed or otherwise configured to implement methods of the disclosure, such as to control the systems described herein (e.g., reagent dispensing, detecting, etc.) and collect, receive, and/or analyze sequencing information. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an isolated or substantially isolated internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server. The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback. The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries, and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine-readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610. The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as the main memory of a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, results of a nucleic acid sequence (e.g., sequence reads). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, perform error correction on processed sequencing signals.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1: A method, comprising: contacting a growing strand hybridized to a template with a first reagent mixture comprising labeled, non-terminated bases and reversibly terminated bases of a first same canonical base type and detecting a first signal indicative of incorporation of at least a subset of the labeled, non-terminated bases of the first reagent mixture in the growing strand, or lack thereof, to generate first sequencing data; reversing termination of the reversibly terminated bases of the first reagent mixture incorporated in the growing strand, if any; contacting the growing strand with a second reagent mixture comprising labeled, non-terminated bases and terminated bases of the first same canonical base type and detecting a second signal indicative of incorporation of at least a subset of the labeled, non-terminated bases of the second reagent mixture in the growing strand, or lack thereof, to generate second sequencing data; and processing the first sequencing data and the second sequencing data to determine length information of a homopolymer sequence in the template.

Embodiment 2: The method of embodiment 1, wherein the length information of the homopolymer sequence in the template comprises a minimum length of the homopolymer sequence. Embodiment 3: The method of any of embodiments 1 or 2, wherein the length information of the homopolymer sequence in the template comprises a total length of the homopolymer sequence. Embodiment 4: The method of any of embodiments 1-3, further comprising (e) reversing termination of the reversibly terminated bases of the second reagent mixture incorporated in the growing strand, if any, and (f) contacting the growing strand with a third reagent mixture comprising unlabeled, non-terminated bases of the first same canonical base type. Embodiment 5: The method of embodiment 4, further comprising (g) repeating (a)-(f) with a second same canonical base type different from the first canonical base type. Embodiment 6: The method of embodiment 5, further comprising (h) repeating (a)-(f) with a third same canonical base type different from the first canonical base type and the second canonical base type. Embodiment 7: The method of embodiment 6, further comprising (i) repeating (a)-(f) with a fourth same canonical base type different from the first canonical base type, the second canonical base type, and the third canonical base type. Embodiment 8: The method of embodiment 7, further comprising repeating (a)-(i) at least 10 times. Embodiment 9: The method of any of embodiments 1-8, wherein the first signal is localized to a single molecule of the template. Embodiment 10: The method of any of embodiments 1-8, wherein the first signal is localized to a colony of molecules comprising the template. Embodiment 11: The method of any of embodiments 1-10, wherein the template is immobilized to a substrate surface. Embodiment 12: The method of embodiment 11, wherein the template is coupled to a bead that is immobilized to the substrate surface. Embodiment 13: The method of any of embodiments 11-12, wherein the substrate surface comprises at least 1,000,000 individually addressable locations and the template is immobilized to an individually addressable location in the at least 1,000,000 individually addressable locations.

Embodiment 14: A method of sequencing a template molecule, comprising: hybridizing a primer to a primer binding site on the template molecule; extending the primer through a first region of the template molecule, wherein the extending comprises alternatively adding nucleotides and detecting incorporation of nucleotides into the extending primer; denaturing the extended primer from the template molecule; hybridizing a primer to the primer binding site on the template molecule; extending the primer through the first region of the template molecule without detecting incorporation of nucleotides; and extending the primer through a second region of the template molecule, wherein the extending comprises alternatively adding nucleotides and detecting incorporation of nucleotides into the extending primer.

Embodiment 15: The method of embodiment 1, wherein the nucleotides added during the extending (b) and (f) comprise reversibly terminated, labeled nucleotides. Embodiment 16: The method of any one of embodiments 1-2, wherein the nucleotides added during the extending (e) comprise unlabeled nucleotides. Embodiment 17: The method of any one of embodiments 1-2, wherein the nucleotides added during the extending (e) comprise labeled nucleotides. Embodiment 18: The method of any one of embodiments 1-17, wherein the nucleotides added during the extending (e) comprise unterminated nucleotides. Embodiment 19: The method of any one of embodiments 1-18, wherein alternatively adding nucleotides and detecting incorporation of nucleotides in the extending (b) and (f) comprises, in each step, adding nucleotides of more than one base type. Embodiment 20: The method of embodiment 19, further comprising adding nucleotides of the four canonical base types. Embodiment 21: The method of any one of embodiments 19-20, wherein nucleotides of each base type are differently labeled. Embodiment 22: The method of embodiment 21, wherein a nucleotide of a first base type is labeled with a first fluorophore and a nucleotide of a second base type is labeled with a second fluorophore. Embodiment 23: The method of any one of embodiments 19-22, wherein at least one base type of nucleotide is unlabeled. Embodiment 24: The method of any one of embodiments 1-23, wherein at least a subset of the nucleotides added during the extending (b) and (f) comprise unlabeled and/or unterminated nucleotides. Embodiment 25: The method of any one of embodiments 1-24, wherein the extending (b) and (f) further comprise, after detecting incorporation of nucleotides, cleaving reversible terminators from incorporated nucleotides.

Embodiment 26: A method of sequencing a template molecule, comprising: hybridizing a primer to a first primer binding site on the template molecule; extending the primer through a first region of the template molecule, wherein the extending comprises alternatively adding nucleotides and detecting incorporation of nucleotides; extending the primer through a second region of the template molecule, thereby producing a copied template molecule, wherein the extending comprises adding nucleotides of at least one base type and, at one or more time points, detecting incorporation of nucleotides; denaturing the copied template molecule from the template molecule; hybridizing a primer to a second primer binding site on the copied template molecule; extending the primer through a first region of the copied template molecule, wherein the extending comprises alternatively adding nucleotides and detecting incorporation of nucleotides; and extending the primer through a second region of the copied template molecule, wherein the extending comprises adding nucleotides of at least one base type and, at one or more time points, detecting incorporation of nucleotides.

Embodiment 27: The method of embodiment 26, wherein the extending (c) and (g) comprises, in one or more steps, adding nucleotides of two base types. Embodiment 28: The method of embodiment 26, wherein the extending (c) and (g) comprises, in one or more steps, adding nucleotides of three base types. Embodiment 29: The method of embodiment 26, wherein the extending (c) and (g) comprises, in one or more steps, adding nucleotides of four base types. Embodiment 30: The method of any one of embodiments 26-29, wherein the sequence of the first region of the template molecule is determined from detection of nucleotide incorporation in the extending (b) and by at least one detection of nucleotide incorporation in the extending (f). Embodiment 31: The method of embodiment 30, wherein the sequence of the second region of the template molecule is determined from detection of nucleotide incorporation in the extending (f) and by at least one detection of nucleotide incorporation in the extending (b). Embodiment 32: The method of any one of embodiments 26-31, wherein each detection determines a base type of the respective incorporated nucleotide. Embodiment 33: The method of embodiment 32, wherein each detection further comprises a confidence value of a respective nucleotide incorporation. Embodiment 34: The method of any one of embodiments 26-33, wherein the first primer binding site is at 3′ end of the template molecule. Embodiment 35: The method of any one of embodiments 26-34, wherein the second primer binding site is at 3′ end of the copied template molecule. Embodiment 36: The method of any one of embodiments 26-35, wherein the template molecule and the copied template molecule are each single-stranded. Embodiment 37: The method of any one of embodiments 26-37, wherein the nucleotides added during the extending (b) and (f) comprise reversibly terminated, labeled nucleotides. Embodiment 38: The method of any one of embodiments 26-38, wherein the nucleotides added during the extending (c) and (g) comprise a first subset of unlabeled nucleotides and a second subset of labeled nucleotides. Embodiment 39: The method of any one of embodiments 26-38, wherein the nucleotides added during the extending (c) and (g) comprise labeled nucleotides. Embodiment 40: The method of any one of embodiments 26-38, wherein the nucleotides added during the extending (c) and (g) comprise unterminated nucleotides. Embodiment 41: The method of any one of embodiments 26-40, wherein at least a subset of the nucleotides added during the extending (b) and (f) comprise unlabeled and/or unterminated nucleotides. Embodiment 42: The method of any one of embodiments 26-41, wherein the extending (b) and (f) further comprise, after detecting incorporation of nucleotides, cleaving reversible terminators from incorporated nucleotides.

Embodiment 43: A method of sequencing a nucleic acid molecule, comprising: hybridizing the nucleic acid molecule to a primer to form a hybridized template; extending the primer using labeled, terminated nucleotides provided in multiple flows comprising four nucleotide base types; detecting a signal from an incorporated labeled nucleotide or an absence of a signal as the primer is extended by the nucleotide flows; denaturing the extended primer; hybridizing another primer to the nucleic acid molecule to a primer to reform the hybridized template; extending the primer using unterminated nucleotide provided in separate nucleotide flows according to a repeated flow-cycle order comprising four or more separate nucleotide flows; further extending the primer using labeled, terminated nucleotides provided in multiple flows comprising four nucleotide base types; and detecting a signal from an incorporated labeled nucleotide or an absence of a signal as the primer is extended by the nucleotide flows.

Embodiment 44: A method comprising: providing a plurality of template molecules, wherein the template molecules comprise primers hybridized thereto; contacting the plurality of template molecules with a reaction mixture comprising a plurality of nucleotides under conditions sufficient for one or more nucleotides to be incorporated into one or more primers hybridized to template molecules and for one or more nucleotides to be transiently bound to one or more template molecules; and detecting a signal, wherein the signal indicates the presence or absence of incorporated or transiently bound nucleotides. Embodiment 45: The method of embodiment 44, further comprising: removing transiently bound nucleotides; and detecting an additional signal, wherein the additional signal indicates the presence or absence of incorporated nucleotides.

Embodiment 46: A method comprising: providing a plurality of template molecules with primers hybridized thereto; contacting the plurality of template molecules with a reaction mixture comprising terminated and unterminated nucleotides, wherein a first subset of the plurality of template molecules incorporate a terminated nucleotide and a second subset of the plurality of template molecules transiently bind to an unterminated nucleotide; detecting a signal, wherein the signal indicates the presence or absence of incorporated or transiently bound nucleotides; removing transiently bound nucleotides; and detecting an additional signal, wherein the additional signal indicates the presence or absence of incorporated nucleotides.

Embodiment 47: The method of any one of embodiments 44-46, wherein the reaction mixture further comprises a polymerase that can incorporate terminated nucleotides and transiently bind unterminated nucleotides. Embodiment 48: The method of any one of embodiments 44-46, wherein the reaction mixture further comprises a first polymerase that can incorporate terminated nucleotides and a second polymerase that can transiently bind unterminated nucleotides. Embodiment 49: The method of any one of embodiments 44-46, wherein the reaction mixture further comprises a polymerase that can incorporate nucleotides and catalyze transient binding of nucleotides. Embodiment 50: The method of any one of embodiments 44-49, wherein the transiently bound nucleotides are non-incorporable nucleotides. Embodiment 51: The method of any one of embodiments 44-50, wherein the incorporated nucleotides are reversibly terminated nucleotides. Embodiment 52: The method of any one of embodiments 44-51, wherein the reaction mixture comprises: a nucleotide of a first base type comprising a first label type; a nucleotide of a second base type comprising a second label type; a nucleotide of a third base type comprising the first label type; and a nucleotide of a fourth base type comprising the second label type. Embodiment 53: The method of embodiment 52, wherein nucleotides of the first and second base types are reversibly terminated. Embodiment 54: The method of embodiment 52 or embodiment 53, wherein nucleotides of the third and fourth base types are non-incorporable. Embodiment 55: The method of any one of embodiments 44-50, wherein the reaction mixture comprises: a nucleotide of a first base type comprising a first label type; a nucleotide of a second base type comprising a second label type; a nucleotide of a third base type; and a nucleotide of a fourth base type comprising the second label type. Embodiment 56: The method of embodiment 55, wherein the nucleotide of the third base type does not comprise a label. Embodiment 57: The method of embodiment 55 or embodiment 56, wherein nucleotides of the first, second, and third base types are reversibly terminated. Embodiment 58: The method of any one of embodiments 55-57, wherein nucleotides of the fourth base type are non-incorporable. Embodiment 59: The method of any one of embodiments 45-58, further comprising, after the detecting (e): contacting the plurality of template molecules with another reaction mixture comprising a plurality of terminated nucleotides; and cleaving reversible terminator and labeling moieties. Embodiment 60: The method of embodiment 59, further comprising repeating steps (a)-(g) one or more times to determine the sequences of the template molecules.

Embodiment 61: A method for identifying a sequence, comprising: providing a template molecule with a primer hybridized thereto; performing at least two repeated cycles of incorporation or transient binding of nucleotides to the template molecule; detection of a signal, wherein the signal indicates the presence or absence of incorporated or transiently bound nucleotides; and cleavage of reversible terminator and labeling moieties; and determining the sequence of the template molecule based on the detection of labeled nucleotides.

Embodiment 62: The method of embodiment 61, wherein the at least two repeated cycles further comprise providing a reaction mixture comprising: a nucleotide of a first base type that comprising a first label type; a nucleotide of a second base type comprising a second label type; a nucleotide of a third base type comprising the first label type; and a nucleotide of a fourth base type comprising the second label type. Embodiment 63: The method of embodiment 62, wherein nucleotides of the first and second base types are reversibly terminated. Embodiment 64: The method of embodiment 62 or embodiment 63, wherein nucleotides of the third and fourth base types are non-incorporable. Embodiment 65: The method of any one of embodiments 61-64, further comprising, prior to cleaving (iii): washing the template molecule to remove transiently bound nucleotides; and detecting an additional signal, wherein the additional signal indicates the presence or absence of incorporated or transiently bound nucleotides. Embodiment 66: The method of any one of embodiments 61-65, further comprising, prior to cleaving (iii): contacting the template molecule with another reaction mixture comprising a plurality of terminated nucleotides.

Embodiment 67: A method comprising: providing a plurality of template molecules with primers hybridized thereto; contacting the plurality of template molecules with a first reaction mixture comprising terminated and unterminated nucleotides, wherein a first subset of the plurality of template molecules incorporate a terminated nucleotide and a second subset of the plurality of template molecules transiently bind to an unterminated nucleotide; detecting a signal, wherein the signal indicates the presence or absence of incorporated or transiently bound nucleotides; removing transiently bound nucleotides; contacting the plurality of template molecules with a second reaction mixture comprising unterminated nucleotides that may transiently bind template molecules; and detecting an additional signal, wherein the additional signal indicates the presence or absence of incorporated or transiently bound nucleotides.

Embodiment 68: The method of embodiment 67, wherein the first reaction mixture further comprises a polymerase that can incorporate terminated nucleotides and transiently bind unterminated nucleotides. Embodiment 69: The method of embodiment 67, wherein the first reaction mixture further comprises a first polymerase that can incorporate terminated nucleotides and a second polymerase that can transiently bind unterminated nucleotides. Embodiment 70: The method of any one of embodiments 67-69, wherein the transiently bound nucleotides are non-incorporable nucleotides. Embodiment 71: The method of any one of embodiments 67-70, wherein the reaction mixture comprises: nucleotide of a first base type comprising a first label type; a nucleotide of a second base type; a nucleotide of a third base type comprising the first label type; and a nucleotide of a fourth base type comprising a second label type. Embodiment 72: The method of any one of embodiments 46-71, wherein the template molecules are nucleic acid molecules. Embodiment 73: The method of embodiment 72, wherein the nucleic acid molecules are DNA or RNA. Embodiment 74: The method of embodiment 72 or embodiment 73, wherein the template molecules are amplified. Embodiment 75: The method of any one of embodiments 72-74, wherein the template molecules are DNA nanoballs. Embodiment 76: The method of any one of embodiments 72-75, wherein the template molecules are not amplified.

Embodiment 77: A method of sequencing, comprising: providing a template molecule comprising a primer hybridized thereto, wherein the primer is not blocked for nucleotide incorporation; contacting the template molecule with a first reaction mixture comprising a plurality of nucleotides under conditions sufficient to permit binding of non-incorporable nucleotides to the template molecule, wherein the plurality of nucleotides comprises at least one nucleotide base type; and detecting a signal, wherein the signal indicates binding or lack thereof of a nucleotide analog to the template molecule.

Embodiment 78: The method of embodiment 77, further comprising, after the detecting (c), washing the template molecule to remove non-incorporable nucleotides. Embodiment 79: The method of embodiment 77 or embodiment 78, wherein the binding of non-incorporable nucleotides comprises the formation of hydrogen bonds. Embodiment 80: The method of any one of embodiments 77-79, wherein the binding of non-incorporable nucleotides is transient. Embodiment 81: The method of any one of embodiments 77-80, further comprising: contacting the template molecules with a second reaction mixture comprising a plurality of terminated nucleotides under conditions sufficient to permit incorporation of nucleotides into the primer hybridized to the template molecule. Embodiment 82: The method of embodiment 81, further comprising: cleavage of reversible terminators from incorporated nucleotides. Embodiment 83: The method of embodiment 82, further comprising: repeating steps (a)-(e) one or more times to determine the sequence of the template molecule. Embodiment 84: The method of any one of embodiments 81-83, wherein the first reaction mixture further comprises a first polymerase to enable binding of non-incorporable nucleotides to the template molecule, and the second reaction mixture comprises a second polymerase to enable incorporation of terminated nucleotides into the primer. Embodiment 85: The method of embodiment 84, wherein the first and second polymerase are the same. Embodiment 86: The method of embodiment 84, wherein the first and second polymerase are different. Embodiment 87: The method of any one of embodiments 77-86, wherein the first reaction mixture comprises: a nucleotide of a first base type comprising a first label type; a nucleotide of a second base type; a nucleotide of a third base type comprising the first label type; and a nucleotide of a fourth base type comprising a second label type. Embodiment 88: The method of embodiment 87, wherein the nucleotide of the second base type does not comprise a label. Embodiment 89: The method of any one of embodiments 77-88, wherein the first reaction mixture does not comprise a cation that inhibits nucleotide incorporation. Embodiment 90: The method of any one of embodiments 77-89, wherein the first reaction mixture comprises a polymerase that exhibits exonuclease activity. Embodiment 91: The method of any one of embodiments 77-90, wherein the template molecule is a nucleic acid molecule. Embodiment 92: The method of embodiment 91, wherein the nucleic acid molecule is DNA or RNA. Embodiment 93: The method of embodiment 91 or embodiment 92, wherein the template molecule is amplified. Embodiment 94: The method of any one of embodiments 91-93, wherein the template molecule is a DNA nanoball. Embodiment 95: The method of any one of embodiments 91-94, wherein the template molecule is not amplified.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1: Extended Sequencing Flow Orders

Flow cycle orders need not be limited to four base flow cycles (e.g., one each of A, G, C, and T, in any repeated order), and may be an extended flow cycle with more than four base types in a cycle. The extended cycle order may be repeated for the desired number of cycles to extend the sequencing primer. By way of example, in some embodiments, the extended flow order includes 5, 6, 7, 8, 9, 10, 11, 12, 13, 14, 15, 16, 17, 18, 19, 20 or more separate nucleotide flows in the flow cycle order. The cycles can include at least one each of A, G, C, and T, but repeat one or more base types within the cycle before the cycle is repeated.

The extended flow cycle orders can be useful for detecting a greater proportion of small genomic variants (e.g., SNPs) than a flow cycle order with four repeated bases. For example, there are 192 valid configurations of substitution SNPs in the form XYZ→XQZ where QAY (and Q, X, Y, and Z are each any one of A, C, G, and T). Of these, 168 can produce a new signal (i.e., a new non-zero signal or a new zero signal) in the sequencing data set (e.g., a flowgram). A new zero or non-zero signal combined with a sensitive flow order can produce a signal that is propagated for multiple flow positions (e.g., a flow shift or cycle shift, which may extend more than the length of the cycle), given identical trailing sequences in the variant relative to the reference. It is noted that insertion or deletion of a homopolymer, rather than a homopolymer length change, can result in a signal difference propagation. The remaining 24 variants cause a homopolymer length change at the affected flow position, but such a change does not cause a propagated signal change. Thus, a theoretical maximum of 87.5% of SNPs can result in a new signal that differs from a reference (or candidate) sequence for more than two flow positions. As discussed above, the propagated signal difference increases the likelihood difference between a test sequencing data set and an incorrectly matched candidate sequence. Further, the propagated signal change depends on the flow order spanning the variant.

Sequencing nucleic acid molecules in a test sample that have been randomly fragmented results in a random shift in the flow order context of the variant when the sequencing primer is extended using the flow order. That is, the flow position of the variant may change depending on the start position of the sequenced nucleic acid molecule. Not all flow cycle combinations are able to detect signal changes at more than two flow positions for all 87.5% of SNPs, even if all sequencing start positions in a nucleic acid molecule sequence are utilized. For example, the four-base flow cycle order T-A-C-G can result in a test sequencing data set that differs from a reference sequencing data set at more than two flow positions for 41.7% of SNPs. As further discussed herein, extended flow cycle orders have been designed so that all of the theoretical maximum of SNPs (i.e., 87.5% of possible SNPs, or all SNPs other than those resulting in a homopolymer length change) can give rise to a difference at more than two flow position between the test sequencing data set and the reference sequencing data set, given a high enough sequencing depth (i.e., sampling a sufficiently large number of start positions).

Extended sequencing flow orders may have different efficiencies (i.e., the average number of incorporations per flow when used to sequence a human reference genome). In some embodiments, the flow order has an efficiency of about 0.6 or greater (such as about 0.62 or greater, about 0.64 or greater, about 0.65 or greater, about 0.66 or greater, or about 0.67 or greater). In some embodiments, the flow order has an efficiency of about 0.6 to about 0.7. Examples of flow cycle orders and corresponding estimated efficiencies are shown in Table 1.

In some embodiments, the extended sequencing flow order can be selected to generate signal differences at more than two flow positions between two sequencing data sets (e.g., a test or target sequencing data set and a candidate or reference sequencing data set) associated with nucleic acid molecules differing by a SNP for about 50% to 87.5%, about 50% to about 80%, about 60% to 87.5%, about 70% to 87.5%, about 70% to about 80%, or about 80% to 87.5% of SNP permutations for at least 5%, at least 10%, at least 15%, at least 20%, at least 25%, or at least 30% of random sequencing start positions (i.e., “flow phases”).

In some embodiments, the extended sequencing flow order can be any one of the extended sequencing flow orders in Table 1. “Shift sensitivity” refers to the maximum sensitivity to generate signal differences at more than two flow positions between two sequencing data sets (e.g., a test or target sequencing data set and a candidate or reference sequencing data set) over all possible SNP permutations. “Maximum shift sensitivity” refers to refers to the maximum sensitivity to generate signal differences at more than two flow positions between two sequencing data sets (e.g., a test or target sequencing data set and a candidate or reference sequencing data set) over all possible SNP permutations at the highest fraction of flow phases at which that sensitivity is maintained.

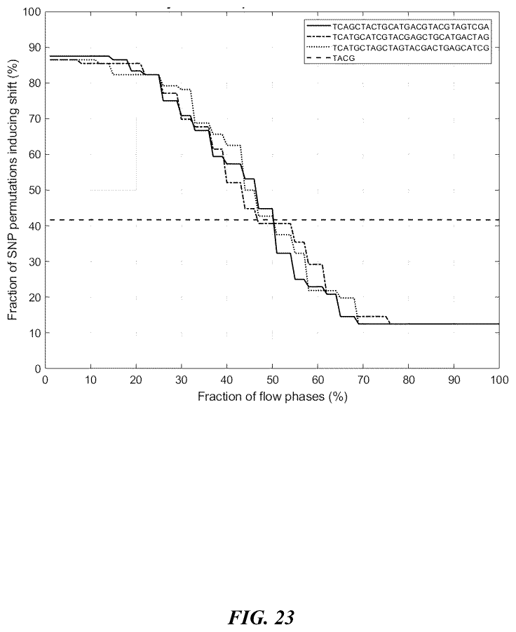

More than a million extended sequencing flow orders were tested in silico for their likelihood to induce a signal change in more than two flow positions over the set of all possible SNPs (XYZ→XQZ where Q+Y (and Q, X, Y, and Z are each any one of A, C, G, and T)). Extended flow orders were designed to have a minimum of 12 base sequences with all valid 2 base flow permutations, and flow orders having sequential base repeats were removed. All possible starting positions for the flow order were tested to assess sensitivity of the extended flow orders to induce the signal change at more than two flow positions. FIG. 23 and Table 1 show exemplary results of this analysis. In FIG. 23, the x-axis indicates the fraction of the flow phases (or fragmentation start positions), and the y-axis indicates the fraction of SNP permutations having induced a signal change at more than two flow positions. Several flow orders induce two or more signal differences at all possible (87.5%) SNP permutations for approximately 10% of reads (or flow start positions). A four base periodic flow order only induces cycle shifts in only 42% of possible SNPs but it does this with all reads or flow phases. A final evaluation of efficiency was performed against a million base subset of human reference genome to establish viability. This is a practical measure of how efficiently the flow order extends the sequence given the patterns and biases in a real organism.

TABLE 1

| | | | Shift | Shift | Shift | Shift |
|---|---|---|---|---|---|---|
| | | | Sensi- | Sensi- | Sensi- | Sensi- |
| | Es- | Maximum | tivity | tivity | tivity | tivity |
| | timated | Shift | @ 5% of | @ 10% of | @ 20% of | @ 30% of |
| | Effi- | Sensi- | Flow | Flow | Flow | Flow |
| Flow Cycle Order | ciency | tivity | Phases | Phases | Phases | Phases |
| T-C-A-G-A-T-G-C-A-T-G-C-T-A-C-G | 67.5% | 82.3% @ | 82.3% | 82.3% | 75.0% | 66.7% |
| | | 19% | | | | |
| T-C-A-C-G-A-T-G-C-A-T-G-C-T-A-G | 67.5% | 83.3% @ | 83.3% | 83.3% | 72.9% | 62.5% |
| | | 12% | | | | |
| T-C-A-T-G-C-A-T-G-C-T-A-C-G-A-G | 67.3% | 82.3% @ | 82.3% | 82.3% | 72.9% | 67.7% |
| | | 12% | | | | |
| T-C-A-G-T-A-C-G-A-T-G-C-A-T-G-C | 67.3% | 82.3% @ | 82.3% | 82.3% | 75.0% | 63.5% |
| | | 12% | | | | |
| T-C-A-G-T-C-G-A-T-G-A-C-T-A-G-C | 67.2% | 81.3% @ | 81.3% | 81.3% | 74.0% | 69.8% |
| | | 12% | | | | |
| T-C-A-T-C-G-A-C-T-G-A-G-C-T-A-G | 67.2% | 81.3% @ | 81.3% | 81.3% | 74.0% | 69.8% |
| | | 12% | | | | |
| T-C-G-T-A-G-C-T-G-A-C-A-T-G-C-A | 67.2% | 83.3% @ | 83.3% | 83.3% | 75.0% | 67.7% |
| | | 12% | | | | |
| T-C-G-T-A-G-C-A-T-G-C-T-A-C-G-A | 67.0% | 79.2% @ | 79.2% | 79.2% | 79.2% | 75.0% |
| | | 25% | | | | |
| T-C-A-T-G-C-A-G-T-C-G-A-C-T-A-G | 66.9% | 83.3% @ | 83.3% | 83.3% | 75.0% | 68.8% |
| | | 19% | | | | |
| T-C-A-T-G-C-A-T-C-G-T-A-C-G-A-G- | 66.7% | 86.5% @ | 86.5% | 85.4% | 85.4% | 69.8% |
| C-T-G-C-A-T-G-A-C-T-A-G | | 7% | | | | |
| T-C-G-A-C-T-G-T-A-G-C-T-A-G-C-A | 66.7% | 82.3% @ | 82.3% | 82.3% | 75.0% | 66.7% |
| | | 19% | | | | |
| T-C-A-C-G-A-T-G-C-T-A-G-C-T-A-G | 66.5% | 82.3% @ | 82.3% | 82.3% | 75.0% | 67.7% |
| | | 12% | | | | |
| T-C-A-G-T-A-C-G-A-T-G-C-T-A-C-G | 66.4% | 83.3% @ | 83.3% | 83.3% | 75.0% | 68.8% |
| | | 19% | | | | |
| T-C-G-A-C-T-A-G-C-A-T-G-C-A-T-G | 66.0% | 81.3% @ | 81.3% | 81.3% | 70.8% | 62.5% |
| | | 12% | | | | |
| T-A-C-G | 66.0% | 41.7% @ | 41.7% | 41.7% | 41.7% | 41.7% |
| | | 100% | | | | |
| T-C-A-G-C-T-G-A-C-T-A-G-T-C-A-T- | 65.7% | 87.5% @ | 87.5% | 87.5% | 82.3% | 75.0% |
| G-A-C-T-A-G-C-G-A-T-C-G | | 11% | | | | |
| T-C-T-A-G-C-A-T-G-A-C-T-G-A-C-G | 65.7% | 83.3% @ | 83.3% | 83.3% | 71.9% | 63.5% |
| | | 12% | | | | |
| T-C-G-A-C-T-A-T-G-C-A-T-G-C-A-G | 65.5% | 81.3% @ | 81.3% | 81.3% | 71.9% | 63.5% |
| | | 19% | | | | |
| T-C-G-A-C-T-G-C-A-T-C-G-A-T-G-C- | 65.4% | 87.5% @ | 87.5% | 87.5% | 82.3% | 74.0% |
| A-G-T-A-C-T-A-G | | 12% | | | | |
| T-C-A-C-T-G-A-C-G-T-A-G-C-T-A-T- | 65.3% | 84.4% @ | 84.4% | 84.4% | 83.3% | 76.0% |
| G-C-A-T-C-G-A-G | | 17% | | | | |
| T-C-A-T-G-C-T-A-G-C-T-A-G-T-A-C- | 65.2% | 86.5% @ | 86.5% | 86.5% | 82.3% | 78.1% |
| G-A-C-T-G-A-G-C-A-T-C-G | | 11% | | | | |
| T-C-G-A-T-G-C-A-T-C-G-T-A-C-T-A- | 65.2% | 87.5% @ | 87.5% | 86.5% | 84.4% | 71.9% |
| G-C-A-G-T-G-A-C | | 8% | | | | |
| T-C-A-T-G-A-G-C-T-A-G-C-A-T-C-G- | 65.2% | 87.5% @ | 87.5% | 86.5% | 81.3% | 70.8% |
| T-A-C-T-G-A-C-G | | 8% | | | | |
| T-C-A-G-C-A-T-G-T-A-C-T-G-A-T-G- | 65.0% | 87.5% @ | 87.5% | 87.5% | 82.3% | 77.1% |
| C-A-T-C-G-A-G-C-T-A-C-G | | 11% | | | | |
| T-C-A-G-T-A-C-T-A-G-C-A-T-G-C-G- | 65.0% | 86.5% @ | 86.5% | 86.5% | 78.1% | 74.0% |
| A-T-C-G-T-A-G-C-T-G-A-C | | 11% | | | | |
| T-C-A-C-G-T-A-G-C-T-A-T-G-C-T-G- | 64.6% | 85.4% @ | 85.4% | 84.4% | 76.0% | 61.5% |
| A-C-T-G-A-C-A-T-G-A-C-T-A-G-C-G | | 9% | | | | |
| T-C-A-G-C-T-A-T-G-A-C-T-G-A-G-C- | 64.5% | 85.4% @ | 85.4% | 85.4% | 77.1% | 74.0% |
| A-T-C-G-T-A-C-G | | 12% | | | | |
| T-C-A-G-C-T-A-C-T-G-C-A-T-G-A-C- | 64.5% | 87.5% @ | 87.5% | 87.5% | 83.3% | 70.8% |
| G-T-A-C-G-T-A-G-T-C-G-A | | 14% | | | | |
| T-C-A-G-A-C-T-A-G-C-G-A-T-G-C-A- | 64.5% | 86.5% @ | 86.5% | 86.5% | 83.3% | 62.5% |
| T-G-T-C-T-A-G-T-C-A-C-G | | 11% | | | | |
| T-C-A-T-C-G-A-C-T-G-C-G-A-T-G-C- | 64.4% | 85.4% @ | 85.4% | 85.4% | 83.3% | 72.9% |
| T-A-G-T-A-C-A-G | | 17% | | | | |
| T-C-A-C-G-T-A-C-T-G-A-C-A-T-G-C- | 64.4% | 85.4% @ | 85.4% | 84.4% | 83.3% | 72.9% |
| A-T-G-C-T-A-G-T-A-G-C-G-A-T-C-G | | 9% | | | | |
| T-C-A-G-T-G-C-T-A-C-G-T-C-A-C-G- | 64.4% | 86.5% @ | 86.5% | 86.5% | 71.9% | 67.7% |
| A-T-C-A-G-A-T-G-C-T-A-G | | 11% | | | | |
| T-C-A-G-C-G-A-T-G-A-C-T-A-G-C-T- | 64.4% | 85.4% @ | 85.4% | 85.4% | 84.4% | 66.7% |
| A-C-G-T-C-A-T-G | | 17% | | | | |
| T-C-A-T-G-C-T-A-C-G-A-G | 64.4% | 81.3% @ | 81.3% | 81.3% | 80.2% | 66.7% |
| | | 17% | | | | |
| T-C-A-T-G-A-C-G-T-A-C-G-A-C-T-C- | 64.3% | 85.4% @ | 85.4% | 85.4% | 82.3% | 75.0% |
| A-T-G-C-A-G-T-G-C-T-A-G | | 11% | | | | |
| T-C-A-G-T-C-G-A-T-G-C-T-A-C-T-G- | 64.3% | 87.5% @ | 87.5% | 86.5% | 83.3% | 74.0% |
| C-A-T-A-C-G-T-C-G-A-T-G-A-C-A-G | | 9% | | | | |
| T-C-G-A-T-G-C-T-A-C-A-G | 64.3% | 81.3% @ | 81.3% | 81.3% | 80.2% | 66.7% |
| | | 17% | | | | |
| T-C-A-G-T-C-G-A-C-A-T-G-C-A-T-C- | 64.2% | 87.5% @ | 87.5% | 86.5% | 79.2% | 70.8% |
| G-A-T-A-C-G-T-G-C-T-A-G-C-T-A-G | | 9% | | | | |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A method, comprising:
a. contacting a growing strand hybridized to a template with a first reagent mixture comprising labeled, non-terminated nucleotides and reversibly terminated nucleotides of a first canonical base type and detecting a first signal indicative of incorporation of labeled, non-terminated nucleotides of the first reagent mixture in the growing strand, or lack thereof, to generate first sequencing data;
b. reversing termination of a reversibly terminated nucleotide of the first reagent mixture incorporated in the growing strand;
c. contacting the growing strand with a second reagent mixture comprising labeled, non-terminated nucleotides and reversibly terminated nucleotides of the first canonical base type and detecting a second signal indicative of incorporation of labeled, non-terminated nucleotides of the second reagent mixture in the growing strand, or lack thereof, to generate second sequencing data; and
d. processing the first sequencing data and the second sequencing data to determine a length of a homopolymer sequence in the template.

2. The method of claim 1, wherein the length of the homopolymer sequence in the template comprises a minimum length of the homopolymer sequence.

3. The method of claim 1, wherein the length of the homopolymer sequence in the template comprises a total length of the homopolymer sequence.

4. The method of claim 1, further comprising repeating the (b) reversing termination and the (c) contacting one or more times to generate additional sequencing data; and wherein the length of the homopolymer sequence is determined from the first sequencing data, the second sequencing data, and the additional sequencing data.

5. The method of claim 1, further comprising:
e. reversing termination of a reversibly terminated nucleotide of the second reagent mixture incorporated in the growing strand; and
f. contacting the growing strand with a third reagent mixture comprising non-terminated nucleotides of the first canonical base type.

6. The method of claim 5, further comprising (g) repeating (a)-(f) with a second canonical base type different from the first canonical base type.

7. The method of claim 5, further comprising repeating (a)-(g) at least 10 times according to a flow order.

8. The method of claim 5, wherein the third reagent mixture comprises non-terminated, unlabeled nucleotides.

9. The method of claim 1, wherein the first signal is localized to a single molecule of the template.

10. The method of claim 1, wherein the first signal is localized to a colony of molecules comprising the template.

11. The method of claim 1, wherein the template is immobilized to a substrate surface.

12. The method of claim 11, wherein the template is coupled to a bead that is immobilized to the substrate surface.

13. The method of claim 12, wherein the substrate surface comprises at least 1,000,000 individually addressable locations and the template is immobilized to an individually addressable location of the at least 1,000,000 individually addressable locations.

14. The method of claim 1, wherein the first reagent mixture or the second reagent mixture comprises nucleotides of an additional canonical base type.

15. The method of claim 14, wherein a nucleotide of the first canonical base type is labeled with a first fluorophore and a nucleotide of the additional canonical base type is labeled with a second fluorophore.

16. The method of claim 1, where the first reagent mixture or the second reagent mixture comprises nucleotides of four canonical base types.

17. The method of claim 16, wherein nucleotides of each base type are differently labeled.

18. The method of claim 16, wherein nucleotides of at least one base type are unlabeled.

19. The method of claim 1, wherein the first reagent mixture or the second reagent mixture further comprises unlabeled, non-terminated nucleotides of the first canonical base type.

20. The method of claim 1, further comprising after detecting incorporation of labeled, non-terminated nucleotides, cleaving labels from nucleotides incorporated in the growing strand.
